SEQUENCE LISTING (192 sequences)

SEQ ID NO: 1 - protein, 10 aa, synthetic construct
  REGION 1..10: note = Heavy chain CDR1 of clone 98.
GFSLTRYNVH

SEQ ID NO: 2 - protein, 9 aa, synthetic construct
  REGION 1..9: note = Heavy chain CDR2 of clone 98.
VIWTGGSTD

SEQ ID NO: 3 - protein, 4 aa, synthetic construct
  REGION 1..4: note = Heavy chain CDR3 of clone 98. 2
DLGY

SEQ ID NO: 4 - protein, 16 aa, synthetic construct
  REGION 1..16: note = Light chain CDR1 of clone 98.
KSSQSLKYRDGKTYLN

SEQ ID NO: 5 - protein, 7 aa, synthetic construct
  REGION 1..7: note = Light chain CDR2 of clone 98.
QVSKLDS

SEQ ID NO: 6 - protein, 9 aa, synthetic construct
  REGION 1..9: note = Light chain CDR3 of clone 98.
CQGSYSPHT

SEQ ID NO: 7 - protein, 10 aa, synthetic construct
  REGION 1..10: note = Heavy chain CDR1 of clone 101.
GFSLTSYYMQ

SEQ ID NO: 8 - protein, 9 aa, synthetic construct
  REGION 1..9: note = Heavy chain CDR2 of clone 101.
FIRSGGSTE

SEQ ID NO: 9 - protein, 10 aa, synthetic construct
  REGION 1..10: note = Heavy chain CDR3 of clone 101.
AFYGGYYFDY

SEQ ID NO: 10 - protein, 11 aa, synthetic construct
  REGION 1..11: note = Light chain CDR1 of clone 101.
KASQNVGSNVD

SEQ ID NO: 11 - protein, 7 aa, synthetic construct
  REGION 1..7: note = Light chain CDR2 of clone 101.
KASNRYT

SEQ ID NO: 12 - protein, 8 aa, synthetic construct
  REGION 1..8: note = Light chain CDR3 of clone 101.
MQSNTKYT

SEQ ID NO: 13 - protein, 131 aa, synthetic construct
  REGION 1..131: note = Heavy chain of clone 98.
MAVLVLLLCLVTFPTCVLSQVQLKESGPGLVQPSQTLSLTCTVSGFSLTRYNVHWVRQPTGKGLEWMGVIWTGGSTDYNSALKSRLSISRDTSKSQVFLKMNSLQTEDIATYYCARDLGYWGQGVMVTVSS

SEQ ID NO: 14 - protein, 132 aa, synthetic construct
  REGION 1..132: note = Light chain of clone 98.
MMSPAQFLFLLMLWIQETHSDIVMTQTPLSLSVAIGQSASISCKSSQSLKYRDGKTYLNWVFQSPGQSPKRLIYQVSKLDSGVPDRFSGTGSETDFTLKISRVEAEDLGVYYCCQGSYSPHTFGAGTKLELK

SEQ ID NO: 15 - protein, 137 aa, synthetic construct
  REGION 1..137: note = Heavy chain of clone 101.
MAILVLLLCLVTIPHSVLSQVQLKETGPGLVQPTQTLSITCTVSGFSLTSYYMQWVRQTPGKGLEWMGFIRSGGSTEYNSEFKSRLSISRDTSKNQVFLKMNSLKTEDTGVYYCARAFYGGYYFDYWGQGVMVTVSS

SEQ ID NO: 16 - protein, 130 aa, synthetic construct
  REGION 1..130: note = Light chain of clone 101.
MGIRMESHTRVFIFLLLWLSGADGDIVMTQSPTSISISVGERVTMNCKASQNVGSNVDWYQQKTGQSPKLLIYKASNRYTGVPDRFTGSGSGTDFTFTISNMQAEDLAVYYCMQSNTKYTFGAGTKLELK

SEQ ID NO: 17 - protein, 517 aa, Homo sapiens
  REGION 1..517: note = MISC_FEATURE - TMEM-180 protein.
MGLGQPQAWLLGLPTAVVYGSLALFTTILHNVFLLYYVDTFVSVYKINKMAFWVGETVFLLWNSLNDPLFGWLSDRQFLSSQPRSGAGLSSRAVVLARVQALGWHGPLLALSFLAFWVPWAPAGLQFLLCLCLYDGFLTLVDLHHHALLADLALSAHDRTHLNFYCSLFSAAGSLSVFASYAFWNKEDFSSFRAFCVTLAVSSGLGFLGATQLLRRRVEAARKDPGCSGLVVDSGLCGEELLVGSEEADSITLGRYLRQLARHRNFLWFVSMDLVQVFHCHFNSNFFPLFLEHLLSDHISLSTGSILLGLSYVAPHLNNLYFLSLCRRWGVYAVVRGLFLLKLGLSLLMLLAGPDHLSLLCLFIASNRVFTEGTCKLLTLVVTDLVDEDLVLNHRKQAASALLFGMVALVTKPGQTFAPLLGTWLLCFYTGHDLFQQSLITPVGSAHPWPEPPAPAPAQAPTLRQGCFYLLVLVPITCALLQLFTWSQFTLHGRRLHMVKAQRQNLSQAQTLDVKMV

SEQ ID NO: 18 - DNA, 73 bp, synthetic construct
  misc_feature 1..73: note = Synthesized primer.
  source 1..73: mol_type = other DNA
cagacctgcacctgaacgtggttgagagctgaggaattgacggtcactgagggactgtaatgctgcacttcgc

SEQ ID NO: 19 - DNA, 78 bp, synthetic construct
  misc_feature 1..78: note = Synthesized primer.
  source 1..78: mol_type = other DNA
caaccacgttcaggtgcaggtctgtcttcacgtgctgttgtactggctcgtgttcaagagttcaaactggaggacctg

SEQ ID NO: 20 - DNA, 33 bp, synthetic construct
  misc_feature 1..33: note = Synthesized primer.
  source 1..33: mol_type = other DNA
gagatatacatatgtcggaggtgactcgtagtc

SEQ ID NO: 21 - DNA, 31 bp, synthetic construct
  misc_feature 1..31: note = Synthesized primer.
  source 1..31: mol_type = other DNA
tggtgctcgagaataggctgaacatcaaatg

SEQ ID NO: 22 - DNA, 54 bp, synthetic construct
  misc_feature 1..54: note = Synthesized primer.
  source 1..54: mol_type = other DNA
gcgaagtgcagcattacagtccgatcatctgagtctgctgtgcctcttcattgc

SEQ ID NO: 23 - DNA, 66 bp, synthetic construct
  misc_feature 1..66: note = Synthesized primer.
  source 1..66: mol_type = other DNA
caggtcctccagtttgaactcagaagctgcttgcttacgatgattcagcaccaggtcctcgtctac

SEQ ID NO: 24 - DNA, 33 bp, synthetic construct; identical to SEQ ID NO: 20
  misc_feature 1..33: note = Synthesized primer.
  source 1..33: mol_type = other DNA

SEQ ID NO: 25 - DNA, 31 bp, synthetic construct; identical to SEQ ID NO: 21
  misc_feature 1..31: note = Synthesized primer.
  source 1..31: mol_type = other DNA

SEQ ID NO: 26 - DNA, 45 bp, synthetic construct
  misc_feature 1..45: note = Synthesized primer.
  source 1..45: mol_type = other DNA
ctaatacgactcactatagggcaagcagtggtatcaacgcagagt

SEQ ID NO: 27 - DNA, 22 bp, synthetic construct
  misc_feature 1..22: note = Synthesized primer.
  source 1..22: mol_type = other DNA
ctaatacgactcactatagggc

SEQ ID NO: 28 - DNA, 29 bp, synthetic construct
  misc_feature 1..29: note = Synthesized primer.
  source 1..29: mol_type = other DNA
cccatggccaccarattctyatcagacag

SEQ ID NO: 29 - DNA, 45 bp, synthetic construct; identical to SEQ ID NO: 26
  misc_feature 1..45: note = Synthesized primer.
  source 1..45: mol_type = other DNA

SEQ ID NO: 30 - DNA, 22 bp, synthetic construct
  misc_feature 1..22: note = Synthesized primer.
  source 1..22: mol_type = other DNA
ctaatacgactcactatagggc

SEQ ID NO: 31 - DNA, 28 bp, synthetic construct
  misc_feature 1..28: note = Synthesized primer.
  source 1..28: mol_type = other DNA
gttgttcawgargcacacgactgaggca

SEQ ID NO: 32 - protein, 10 aa, synthetic construct
  REGION 1..10: note = Heavy chain CDR1 of clone 212.
GFTFSDYAMA

SEQ ID NO: 33 - protein, 9 aa, synthetic construct
  REGION 1..9: note = Heavy chain CDR2 of clone 212. 16
TIIYDGSST

SEQ ID NO: 34 - protein, 8 aa, synthetic construct
  REGION 1..8: note = Heavy chain CDR3 of clone 212.
HWYWYFDF

SEQ ID NO: 35 - protein, 11 aa, synthetic construct
  REGION 1..11: note = Light chain CDR1 of clone 212.
LASEGISNDLA

SEQ ID NO: 36 - protein, 7 aa, synthetic construct
  REGION 1..7: note = Light chain CDR2 of clone 212.
AASRLQD

SEQ ID NO: 37 - protein, 9 aa, synthetic construct
  REGION 1..9: note = Light chain CDR3 of clone 212.
QQSYKYPLT

SEQ ID NO: 38 - protein, 136 aa, synthetic construct
  REGION 1..136: note = Heavy chain of clone 212.
MDIRLSLAFLVLFIKGVQCEVQLVESGGGLVQPGNSLKLSCAASGFTFSDYAMAWVRQSPKKGLEWVATIIYDGSSTYYRDSVKGRFTISRDNAKSTLYLQMDSLRSEDTATYYCATHWYWYFDFWGPGTMVTVSS

SEQ ID NO: 39 - protein, 127 aa, synthetic construct
  REGION 1..127: note = Light chain of clone 212.
MGVPTQLLGLLLLWITDAICDIQMTQSPASLSASLGETVSIECLASEGISNDLAWYQQKSGKSPQLLIYAASRLQDGVPSRFSGSGSGTRYSLKISGMQPEDEADYFCQQSYKYPLTFGSGTKLEIK

SEQ ID NO: 40 - protein, 10 aa, synthetic construct
  REGION 1..10: note = Heavy chain CDR1 of clone 212.
GFTFSDYAMA

SEQ ID NO: 41 - protein, 9 aa, synthetic construct
  REGION 1..9: note = Heavy chain CDR2 of clone 212.
TIIYDGSST

SEQ ID NO: 42 - protein, 8 aa, synthetic construct
  REGION 1..8: note = Heavy chain CDR3 of clone 212.
HWYWYFDF

SEQ ID NO: 43 - protein, 11 aa, synthetic construct
  REGION 1..11: note = Light chain CDR1 of clone 212.
LASEGISNDLA

SEQ ID NO: 44 - protein, 7 aa, synthetic construct
  REGION 1..7: note = Light chain CDR2 of clone 212.
AASRLQD

SEQ ID NO: 45 - protein, 9 aa, synthetic construct
  REGION 1..9: note = Light chain CDR3 of clone 212.
QQSYKYPLT

SEQ ID NO: 46 - protein, 136 aa, synthetic construct; identical to SEQ ID NO: 38
  REGION 1..136: note = Heavy chain variable region of clone 212.

SEQ ID NO: 47 - protein, 127 aa, synthetic construct; identical to SEQ ID NO: 39
  REGION 1..127: note = Light chain variable region of clone 212. 22

SEQ ID NO: 48 - protein, 5 aa, synthetic construct
  REGION 1..5: note = Heavy chain CDR1 of clone 129.
DCALN

SEQ ID NO: 49 - protein, 15 aa, synthetic construct
  REGION 1..15: note = Heavy chain CDR2 of clone 129.
WINTQTGKPTYADDF

SEQ ID NO: 50 - protein, 8 aa, synthetic construct
  REGION 1..8: note = Heavy chain CDR3 of clone 129.
EDYGYFDY

SEQ ID NO: 51 - protein, 11 aa, synthetic construct
  REGION 1..11: note = Light chain CDR1 of clone 129.
QASQNINKFIA

SEQ ID NO: 52 - protein, 7 aa, synthetic construct
  REGION 1..7: note = Light chain CDR2 of clone 129.
YTSTLVS

SEQ ID NO: 53 - protein, 8 aa, synthetic construct
  REGION 1..8: note = Light chain CDR3 of clone 129.
LQYDNLRT

SEQ ID NO: 54 - protein, 136 aa, synthetic construct
  REGION 1..136: note = Heavy chain variable region of clone 129.
MDWLWNLLFLMVVAQSAQAQIQLVQSGPELKKPGESVKISCKASGYTFTDCALNWVKQAPGNGLKWMGWINTQTGKPTYADDFKQRFVFSLETSASTAYLQINNLNIEDTATYFCTREDYGYFDYWGQGVMVTVSS

SEQ ID NO: 55 - protein, 126 aa, synthetic construct
  REGION 1..126: note = Light chain variable region of clone 129.
MRTSIQLLGLLLLWLHDAQCDIQMTQSPPSLSASLGDKVTITCQASQNINKFIAWYQQKPGKAPRHLVHYTSTLVSGTPSRFSGSGSGRDYSFSISNVESEDIASYYCLQYDNLRTFGGGTKLELK

SEQ ID NO: 56 - protein, 5 aa, synthetic construct
  REGION 1..5: note = Heavy chain CDR1 of clone 382.
NYGMH

SEQ ID NO: 57 - protein, 15 aa, synthetic construct
  REGION 1..15: note = Heavy chain CDR2 of clone 382.
SISPSGGSTYYRDSV

SEQ ID NO: 58 - protein, 13 aa, synthetic construct
  REGION 1..13: note = Heavy chain CDR3 of clone 382.
SASITAYYYVMDA

SEQ ID NO: 59 - protein, 11 aa, synthetic construct
  REGION 1..11: note = Light chain CDR1 of clone 382.
KASQNVGSNVD

SEQ ID NO: 60 - protein, 7 aa, synthetic construct
  REGION 1..7: note = Light chain CDR2 of clone 382.
KASNRYT

SEQ ID NO: 61 - protein, 9 aa, synthetic construct
  REGION 1..9: note = Light chain CDR3 of clone 382.
MQSNSYPPT

SEQ ID NO: 62 - protein, 141 aa, synthetic construct
  REGION 1..141: note = Heavy chain variable region of clone 382.
MDIRLSLGFLVLFIKGVQCEVQLVESGGGLVQPGRSLKLSCAASGFTFSNYGMHWIRQAPTKGLEWVASISPSGGSTYYRDSVKGRFTISRDNAKSTLYLQMDSLRSEDTATYYCATSASITAYYYVMDAWGQGASVTVSS

SEQ ID NO: 63 - protein, 127 aa, synthetic construct
  REGION 1..127: note = Light chain variable region of clone 382.
MESHTRVFIFLLLWLSGADGDIVMTQSPTSMSISVGDRVTMNCKASQNVGSNVDWYQQKTGQSPKLLIYKASNRYTGVPDRFTGSGSGTDFTFTISNMQAEDLAVYYCMQSNSYPPTFGGGTKLELR

SEQ ID NO: 64 - protein, 5 aa, synthetic construct
  REGION 1..5: note = Heavy chain CDR1 of clone 1361.
NYWMT

SEQ ID NO: 65 - protein, 15 aa, synthetic construct
  REGION 1..15: note = Heavy chain CDR2 of clone 1361.
SITNTGGSTYYPDSV

SEQ ID NO: 66 - protein, 12 aa, synthetic construct
  REGION 1..12: note = Heavy chain CDR3 of clone 1361.
AGYSSYPDYFDY

SEQ ID NO: 67 - protein, 11 aa, synthetic construct
  REGION 1..11: note = Light chain CDR1 of clone 1361.
KAGQNIYNYLA

SEQ ID NO: 68 - protein, 7 aa, synthetic construct
  REGION 1..7: note = Light chain CDR2 of clone 1361.
NANSLQT

SEQ ID NO: 69 - protein, 8 aa, synthetic construct
  REGION 1..8: note = Light chain CDR3 of clone 1361.
QQYSSGWT

SEQ ID NO: 70 - protein, 140 aa, synthetic construct
  REGION 1..140: note = Heavy chain variable region of clone 1361.
MDIRLSLVFLVLFIKGVQCEVQLVESGGGLVQPGRSLKLSCVASGFTFNNYWMTWIRQAPGKGLEWVASITNTGGSTYYPDSVKGRFTISRDYAKSTLYLQMNSLRSEDTATYYCTRAGYSSYPDYFDYWGQGVMLTVSS

SEQ ID NO: 71 - protein, 126 aa, synthetic construct
  REGION 1..126: note = Light chain variable region of clone 1361.
MMAPVQLLGLLLLWLPAMRCNIQMTQSPSLLSASVGDRVTLSCKAGQNIYNYLAWYQQKLGEAPKLLIYNANSLQTGIPSRFSGSGSGTDFTLTISSLQPEDVATYFCQQYSSGWTFGGGTKLELK

SEQ ID NO: 72 - protein, 5 aa, synthetic construct
  REGION 1..5: note = Heavy chain CDR1 of clone 669.
DYWVS

SEQ ID NO: 73 - protein, 16 aa, synthetic construct
  REGION 1..16: note = Heavy chain CDR2 of clone 669.
EIYPNSGATNFNENFK

SEQ ID NO: 74 - protein, 12 aa, synthetic construct
  REGION 1..12: note = Heavy chain CDR3 of clone 669.
DGTMGIAYYFDY

SEQ ID NO: 75 - protein, 11 aa, synthetic construct
  REGION 1..11: note = Light chain CDR1 of clone 669.
KASQNINRYLN

SEQ ID NO: 76 - protein, 7 aa, synthetic construct
  REGION 1..7: note = Light chain CDR2 of clone 669.
NANSLQT

SEQ ID NO: 77 - protein, 9 aa, synthetic construct
  REGION 1..9: note = Light chain CDR3 of clone 669.
LQHNSWPYT

SEQ ID NO: 78 - protein, 139 aa, synthetic construct
  REGION 1..139: note = Heavy chain variable region of clone 669.
MGWICIIFLVATATGVHSQVKLLQSGAALVKPGASVKMSCKASGYTFTDYWVSWVKQSHGKSLEWIGEIYPNSGATNFNENFKGKATLTVDKSTSTAYMELSRLTSEDSAIYYCTRDGTMGIAYYFDYWGQGVMVTVSS

SEQ ID NO: 79 - protein, 127 aa, synthetic construct
  REGION 1..127: note = Light chain variable region of clone 669.
MMAAVQLLGLLLLWVPAMRCDIQMTQSPSFLSASVGDRVTINCKASQNINRYLNWYQQKLGEAPKLLIYNANSLQTGIPSRFSGSGSGTDFTLTISSLQPEDVATYFCLQHNSWPYTFGAGTKLELK

SEQ ID NO: 80 - protein, 5 aa, synthetic construct
  REGION 1..5: note = Heavy chain CDR1 of clone 699.
SYDIS

SEQ ID NO: 81 - protein, 18 aa, synthetic construct
  REGION 1..18: note = Heavy chain CDR2 of clone 699.
AINPGSGGTGYNEKFKGK

SEQ ID NO: 82 - protein, 11 aa, synthetic construct
  REGION 1..11: note = Heavy chain CDR3 of clone 699.
IHGGYRYWFAY

SEQ ID NO: 83 - protein, 10 aa, synthetic construct
  REGION 1..10: note = Light chain CDR1 of clone 699.
RASSSVSYMH

SEQ ID NO: 84 - protein, 7 aa, synthetic construct
  REGION 1..7: note = Light chain CDR2 of clone 699.
DTSKLAS

SEQ ID NO: 85 - protein, 9 aa, synthetic construct
  REGION 1..9: note = Light chain CDR3 of clone 699.
LQRSSYPPT

SEQ ID NO: 86 - protein, 139 aa, synthetic construct
  REGION 1..139: note = Heavy chain variable region of clone 699.
MEWNWVFLFLLSVTAGVHSQVQLQQSGAELTKPGSSVKISCKASGYTFTSYDISWIKQRPGQALEWIGAINPGSGGTGYNEKFKGKATLTVDKSSSTAFMQLSSLTPEDTAVYYCARIHGGYRYWFAYWGQGTLVTVSS

SEQ ID NO: 87 - protein, 128 aa, synthetic construct
  REGION 1..128: note = Light chain variable region of clone 699. 40
MDFQVQSFSLLLISITVIVSSGEIVLTQSPTTMAASPGEKVTITCRASSSVSYMHWFQQKSGTSPKPWIYDTSKLASGVPDRFSGSGSGTSYSLTISSMEAEDAATYYCLQRSSYPPTFGAGTKLELK

SEQ ID NO: 88 - protein, 5 aa, synthetic construct
  REGION 1..5: note = Heavy chain CDR1 of clone 1052.
SNGVG

SEQ ID NO: 89 - protein, 16 aa, synthetic construct
  REGION 1..16: note = Heavy chain CDR2 of clone 1052.
TIWTGGGTNYNSGVQS

SEQ ID NO: 90 - protein, 7 aa, synthetic construct
  REGION 1..7: note = Heavy chain CDR3 of clone 1052.
EYMGFDY

SEQ ID NO: 91 - protein, 11 aa, synthetic construct
  REGION 1..11: note = Light chain CDR1 of clone 1052.
KASQNVGINVG

SEQ ID NO: 92 - protein, 7 aa, synthetic construct
  REGION 1..7: note = Light chain CDR2 of clone 1052.
WASNRDT

SEQ ID NO: 93 - protein, 9 aa, synthetic construct
  REGION 1..9: note = Light chain CDR3 of clone 1052.
LQHNSYPRT

SEQ ID NO: 94 - protein, 134 aa, synthetic construct
  REGION 1..134: note = Heavy chain variable region of clone 1052.
MAVLVLLLCLVTFPSCVLSQVQLKESGPGLMQPSETLSLTCTVSGFSLTSNGVGWVRQPLGKGLVWMGTIWTGGGTNYNSGVQSRLSISRDTSKSQVFLKMNSLQPEDTGTYYCAREYMGFDYWGQGVMVTVSS

SEQ ID NO: 95 - protein, 127 aa, synthetic construct
  REGION 1..127: note = Light chain variable region of clone 1052.
MELISQVFVFLLLWLSGVYGNTVMTQSPTSMFTSVGDRVTMSCKASQNVGINVGWYQQKTGQSPKRLIYWASNRDTGVPDRFTGSGSGTDFTLTISNMQAEDPAIYYCLQHNSYPRTFGGGTKLELK

SEQ ID NO: 96 - protein, 5 aa, synthetic construct
  REGION 1..5: note = Heavy chain CDR1 of clone 1105.
SNGVG

SEQ ID NO: 97 - protein, 16 aa, synthetic construct
  REGION 1..16: note = Heavy chain CDR2 of clone 1105.
TIWSGGGTNYNSAVQS

SEQ ID NO: 98 - protein, 7 aa, synthetic construct
  REGION 1..7: note = Heavy chain CDR3 of clone 1105.
EEKGFAY

SEQ ID NO: 99 - protein, 11 aa, synthetic construct
  REGION 1..11: note = Light chain CDR1 of clone 1105.
KASQNVGINVG

SEQ ID NO: 100 - protein, 7 aa, synthetic construct
  REGION 1..7: note = Light chain CDR2 of clone 1105.
WASNRDT

SEQ ID NO: 101 - protein, 9 aa, synthetic construct
  REGION 1..9: note = Light chain CDR3 of clone 1105.
LQHNSYPRA

SEQ ID NO: 102 - protein, 134 aa, synthetic construct
  REGION 1..134: note = Heavy chain variable region of clone 1105.
MAVLVLLLCLVTFPSCVLSQVQLKESGPGLMQPSETLSLTCTVSGFSLTSNGVGWVRQPLGKGLVWMGTIWSGGGTNYNSAVQSRLSITRDTSKSQVFLKMNSLQPEDTGTYYCAREEKGFAYWGQGTLVTVSS

SEQ ID NO: 103 - protein, 127 aa, synthetic construct
  REGION 1..127: note = Light chain variable region of clone 1105.
MELISQVFVFLLLWLSGVYGNIVMTQSPTSMSTSVGDRVTMSCKASQNVGINVGWYQQKTGQSPKRLIYWASNRDTGVPDRFTGSGSGTDFTLTISNMQAEDPAIYYCLQHNSYPRAFGGGTKLELR

SEQ ID NO: 104 - protein, 10 aa, synthetic construct
  REGION 1..10: note = Predicted HLA type A2-binding peptide derived from TMEM-180 protein.
CLYDGFLTLV

SEQ ID NO: 105 - protein, 10 aa, synthetic construct
  REGION 1..10: note = Predicted HLA type A2-binding peptide derived from TMEM-180 protein.
FLLYYVDTFV

SEQ ID NO: 106 - protein, 10 aa, synthetic construct
  REGION 1..10: note = Predicted HLA type A2-binding peptide derived from TMEM-180 protein.
FLWFVSMDLV

SEQ ID NO: 107 - protein, 10 aa, synthetic construct
  REGION 1..10: note = Predicted HLA type A2-binding peptide derived from TMEM-180 protein. 49
FLSLCRRWGV

SEQ ID NO: 108 - protein, 10 aa, synthetic construct
  REGION 1..10: note = Predicted HLA type A2-binding peptide derived from TMEM-180 protein.
FLLKLGLSLL

SEQ ID NO: 109 - protein, 10 aa, synthetic construct
  REGION 1..10: note = Predicted HLA type A2-binding peptide derived from TMEM-180 protein.
KLLTLVVTDL

SEQ ID NO: 110 - protein, 10 aa, synthetic construct
  REGION 1..10: note = Predicted HLA type A2-binding peptide derived from TMEM-180 protein.
ALLFGMVALV

SEQ ID NO: 111 - protein, 10 aa, synthetic construct
  REGION 1..10: note = Predicted HLA type A2-binding peptide derived from TMEM-180 protein.
ALFTTILHNV

SEQ ID NO: 112 - protein, 10 aa, synthetic construct
  REGION 1..10: note = Predicted HLA type A2-binding peptide derived from TMEM-180 protein.
WLLGLPTAVV

SEQ ID NO: 113 - protein, 10 aa, synthetic construct
  REGION 1..10: note = Predicted HLA type A2-binding peptide derived from TMEM-180 protein.
QLFTWSQFTL

SEQ ID NO: 114 - protein, 10 aa, synthetic construct
  REGION 1..10: note = Predicted HLA type A2-binding peptide derived from TMEM-180 protein.
LALSFLAFWV

SEQ ID NO: 115 - protein, 10 aa, synthetic construct
  REGION 1..10: note = Predicted HLA type A2-binding peptide derived from TMEM-180 protein.
KLGLSLLMLL

SEQ ID NO: 116 - protein, 10 aa, synthetic construct
  REGION 1..10: note = Predicted HLA type A2-binding peptide derived from TMEM-180 protein.
GLGQPQAWLL

SEQ ID NO: 117 - protein, 10 aa, synthetic construct
  REGION 1..10: note = Predicted HLA type A2-binding peptide derived from TMEM-180 protein.
TLHGRRLHMV

SEQ ID NO: 118 - protein, 10 aa, synthetic construct
  REGION 1..10: note = Predicted HLA type A2-binding peptide derived from TMEM-180 protein.
LLWNSLNDPL

SEQ ID NO: 119 - protein, 10 aa, synthetic construct
  REGION 1..10: note = Predicted HLA type A2-binding peptide derived from TMEM-180 protein.
SLNDPLFGWL

SEQ ID NO: 120 - protein, 10 aa, synthetic construct
  REGION 1..10: note = Predicted HLA type A2-binding peptide derived from TMEM-180 protein.
TLVDLHHHAL

SEQ ID NO: 121 - protein, 10 aa, synthetic construct
  REGION 1..10: note = Predicted HLA type A2-binding peptide derived from TMEM-180 protein.
KMAFWVGETV

SEQ ID NO: 122 - protein, 10 aa, synthetic construct
  REGION 1..10: note = Predicted HLA type A2-binding peptide derived from TMEM-180 protein.
YLLVLVPITC

SEQ ID NO: 123 - protein, 10 aa, synthetic construct
  REGION 1..10: note = Predicted HLA type A2-binding peptide derived from TMEM-180 protein. 54
NLSQAQTLDV

SEQ ID NO: 124 - protein, 10 aa, synthetic construct
  REGION 1..10: note = Predicted HLA type A2-binding peptide derived from TMEM-180 protein.
LLTLVVTDLV

SEQ ID NO: 125 - protein, 10 aa, synthetic construct
  REGION 1..10: note = Predicted HLA type A2-binding peptide derived from TMEM-180 protein.
LLSDHISLST

SEQ ID NO: 126 - protein, 10 aa, synthetic construct
  REGION 1..10: note = Predicted HLA type A2-binding peptide derived from TMEM-180 protein.
LMLLAGPDHL

SEQ ID NO: 127 - protein, 10 aa, synthetic construct
  REGION 1..10: note = Predicted HLA type A2-binding peptide derived from TMEM-180 protein.
RQLARHRNFL

SEQ ID NO: 128 - protein, 10 aa, synthetic construct
  REGION 1..10: note = Predicted HLA type A2-binding peptide derived from TMEM-180 protein.
CLCLYDGFLT

SEQ ID NO: 129 - protein, 10 aa, synthetic construct
  REGION 1..10: note = Predicted HLA type A2-binding peptide derived from TMEM-180 protein.
LLCFYTGHDL

SEQ ID NO: 130 - protein, 10 aa, synthetic construct
  REGION 1..10: note = Predicted HLA type A2-binding peptide derived from TMEM-180 protein.
VLVPITCALL

SEQ ID NO: 131 - protein, 10 aa, synthetic construct
  REGION 1..10: note = Predicted HLA type A2-binding peptide derived from TMEM-180 protein.
LLAGPDHLSL

SEQ ID NO: 132 - protein, 10 aa, synthetic construct
  REGION 1..10: note = Predicted HLA type A2-binding peptide derived from TMEM-180 protein.
FGWLSDRQFL

SEQ ID NO: 133 - protein, 10 aa, synthetic construct
  REGION 1..10: note = Predicted HLA type A2-binding peptide derived from TMEM-180 protein.
SLCRRWGVYA

SEQ ID NO: 134 - protein, 10 aa, synthetic construct
  REGION 1..10: note = Predicted HLA type A2-binding peptide derived from TMEM-180 protein.
LQLFTWSQFT

SEQ ID NO: 135 - protein, 10 aa, synthetic construct
  REGION 1..10: note = Predicted HLA type A2-binding peptide derived from TMEM-180 protein.
FLSSQPRSGA

SEQ ID NO: 136 - protein, 10 aa, synthetic construct
  REGION 1..10: note = Predicted HLA type A2-binding peptide derived from TMEM-180 protein.
GLGFLGATQL

SEQ ID NO: 137 - protein, 10 aa, synthetic construct
  REGION 1..10: note = Predicted HLA type A2-binding peptide derived from TMEM-180 protein.
GLQFLLCLCL

SEQ ID NO: 138 - protein, 10 aa, synthetic construct
  REGION 1..10: note = Predicted HLA type A2-binding peptide derived from TMEM-180 protein.
ALSAHDRTHL

SEQ ID NO: 139 - protein, 10 aa, synthetic construct
  REGION 1..10: note = Predicted HLA type A2-binding peptide derived from TMEM-180 protein. 59
QLLRRRVEAA

SEQ ID NO: 140 - protein, 10 aa, synthetic construct
  REGION 1..10: note = Predicted HLA type A2-binding peptide derived from TMEM-180 protein.
VVYGSLALFT

SEQ ID NO: 141 - protein, 10 aa, synthetic construct
  REGION 1..10: note = Predicted HLA type A2-binding peptide derived from TMEM-180 protein.
LLVLVPITCA

SEQ ID NO: 142 - protein, 10 aa, synthetic construct
  REGION 1..10: note = Predicted HLA type A2-binding peptide derived from TMEM-180 protein.]
LLFGMVALVT

SEQ ID NO: 143 - protein, 10 aa, synthetic construct
  REGION 1..10: note = Predicted HLA type A2-binding peptide derived from TMEM-180 protein.
SMDLVQVFHC

SEQ ID NO: 144 - protein, 10 aa, synthetic construct
  REGION 1..10: note = Predicted HLA type A2-binding peptide derived from TMEM-180 protein.
ALLADLALSA

SEQ ID NO: 145 - protein, 10 aa, synthetic construct
  REGION 1..10: note = Predicted HLA type A2-binding peptide derived from TMEM-180 protein.
YAVVRGLFLL

SEQ ID NO: 146 - protein, 10 aa, synthetic construct
  REGION 1..10: note = Predicted HLA type A2-binding peptide derived from TMEM-180 protein.
YKINKMAFWV

SEQ ID NO: 147 - protein, 10 aa, synthetic construct
  REGION 1..10: note = Predicted HLA type A2-binding peptide derived from TMEM-180 protein.
FNSNFFPLFL

SEQ ID NO: 148 - protein, 10 aa, synthetic construct
  REGION 1..10: note = Predicted HLA type A2-binding peptide derived from TMEM-180 protein.
LVLVPITCAL

SEQ ID NO: 149 - protein, 10 aa, synthetic construct
  REGION 1..10: note = Predicted HLA type A2-binding peptide derived from TMEM-180 protein.
GLSSRAVVLA

SEQ ID NO: 150 - protein, 10 aa, synthetic construct
  REGION 1..10: note = Predicted HLA type A2-binding peptide derived from TMEM-180 protein.
FAPLLGTWLL

SEQ ID NO: 151 - protein, 10 aa, synthetic construct
  REGION 1..10: note = Predicted HLA type A24-binding peptide derived from TMEM-180 protein.
SYVAPHLNNL

SEQ ID NO: 152 - protein, 10 aa, synthetic construct
  REGION 1..10: note = Predicted HLA type A24-binding peptide derived from TMEM-180 protein.
VYAVVRGLFL

SEQ ID NO: 153 - protein, 10 aa, synthetic construct
  REGION 1..10: note = Predicted HLA type A24-binding peptide derived from TMEM-180 protein.
SYAFWNKEDF

SEQ ID NO: 154 - protein, 10 aa, synthetic construct
  REGION 1..10: note = Predicted HLA type A24-binding peptide derived from TMEM-180 protein.
NFLWFVSMDL

SEQ ID NO: 155 - protein, 10 aa, synthetic construct
  REGION 1..10: note = Predicted HLA type A24-binding peptide derived from TMEM-180 protein. 64
FFPLFLEHLL

SEQ ID NO: 156 - protein, 10 aa, synthetic construct
  REGION 1..10: note = Predicted HLA type A24-binding peptide derived from TMEM-180 protein.
LFLLKLGLSL

SEQ ID NO: 157 - protein, 10 aa, synthetic construct
  REGION 1..10: note = Predicted HLA type A24-binding peptide derived from TMEM-180 protein.
TFAPLLGTWL

SEQ ID NO: 158 - protein, 10 aa, synthetic construct
  REGION 1..10: note = Predicted HLA type A24-binding peptide derived from TMEM-180 protein.
VFTEGTCKLL

SEQ ID NO: 159 - protein, 10 aa, synthetic construct
  REGION 1..10: note = Predicted HLA type A24-binding peptide derived from TMEM-180 protein.
NFFPLFLEHL

SEQ ID NO: 160 - protein, 10 aa, synthetic construct
  REGION 1..10: note = Predicted HLA type A24-binding peptide derived from TMEM-180 protein.
AFWVGETVFL

SEQ ID NO: 161 - protein, 10 aa, synthetic construct
  REGION 1..10: note = Predicted HLA type A24-binding peptide derived from TMEM-180 protein.
HFNSNFFPLF

SEQ ID NO: 162 - protein, 10 aa, synthetic construct
  REGION 1..10: note = Predicted HLA type A24-binding peptide derived from TMEM-180 protein.
KLLTLVVTDL

SEQ ID NO: 163 - protein, 10 aa, synthetic construct
  REGION 1..10: note = Predicted HLA type A24-binding peptide derived from TMEM-180 protein.
VFLLYYVDTF

SEQ ID NO: 164 - protein, 10 aa, synthetic construct
  REGION 1..10: note = Predicted HLA type A24-binding peptide derived from TMEM-180 protein.
RQLARHRNFL

SEQ ID NO: 165 - protein, 10 aa, synthetic construct
  REGION 1..10: note = Predicted HLA type A24-binding peptide derived from TMEM-180 protein.
RGLFLLKLGL

SEQ ID NO: 166 - protein, 10 aa, synthetic construct
  REGION 1..10: note = Predicted HLA type A24-binding peptide derived from TMEM-180 protein.
RQNLSQAQTL

SEQ ID NO: 167 - protein, 10 aa, synthetic construct
  REGION 1..10: note = Predicted HLA type A24-binding peptide derived from TMEM-180 protein.
LFTTILHNVF

SEQ ID NO: 168 - protein, 10 aa, synthetic construct
  REGION 1..10: note = Predicted HLA type A24-binding peptide derived from TMEM-180 protein.
SLNDPLFGWL

SEQ ID NO: 169 - protein, 10 aa, synthetic construct
  REGION 1..10: note = Predicted HLA type A24-binding peptide derived from TMEM-180 protein.
VFHCHFNSNF

SEQ ID NO: 170 - protein, 10 aa, synthetic construct
  REGION 1..10: note = Predicted HLA type A24-binding peptide derived from TMEM-180 protein.
LFGWLSDRQF

SEQ ID NO: 171 - protein, 5 aa, synthetic construct
  REGION 1..5: note = Heavy chain CDR1 of clone 1361-5
NYWMT

SEQ ID NO: 172 - protein, 15 aa, synthetic construct
  REGION 1..15: note = Heavy chain CDR2 of clone 1361-5
SITNTGGSTAYPDSV

SEQ ID NO: 173 - protein, 12 aa, synthetic construct
  REGION 1..12: note = Heavy chain CDR3 of clone 1361-5
AGYSSYPDYFDY

SEQ ID NO: 174 - protein, 11 aa, synthetic construct
  REGION 1..11: note = Light chain CDR1 of clone 1361-5
KAGQNIYNYLA

SEQ ID NO: 175 - protein, 7 aa, synthetic construct
  REGION 1..7: note = Light chain CDR2 of clone 1361-5
NANSLQT

SEQ ID NO: 176 - protein, 8 aa, synthetic construct
  REGION 1..8: note = Light chain CDR3 of clone 1361-5
QQYSSGWT

SEQ ID NO: 177 - protein, 140 aa, synthetic construct
  REGION 1..140: note = Heavy chain variable region of clone 1361-5
MDIRLSLVFLVLFIKGVQCEVQLVESGGGLVQPGRSLKLSCVASGFTFNNYWMTWIRQAPGKGLEWVASITNTGGSTAYPDSVKGRFTISRDYAKSTLYLQMNSLRSEDTATYYCTRAGYSSYPDYFDYWGQGVMLTVSS

SEQ ID NO: 178 - protein, 120 aa, synthetic construct
  REGION 1..120: note = Light chain variable region of clone 1361-5
MMAPVQLLGLLLLWLPAMRCNIQMTQSPSLLSASVGDRVTLSCKAGQNIYNYLAWYQQKLGEAPKLLIYNANSLQTGIPSRFSGSGSGTDFTLTISSLQPEDVATYFCQQYSSGWTFGGG

SEQ ID NO: 179 - DNA, 1413 bp, synthetic construct
  misc_feature 1..1413: note = Heavy chain of clone 1361-5
  source 1..1413: mol_type = other DNA
  CDS 1..1413
atggacatcaggctcagcttggttttccttgtccttttcataaaaggtgtccagtgtgaggtgcagctggtggagtctgggggaggcctagtgcagcctggaaggtctctgaaactatcctgtgtagcctctggattcacattcaataactactggatgacctggatccgccaggctccagggaaggggctggagtgggttgcatccattactaatactggtggtagcactgcctatccagactctgtgaagggccgattcactatctccagagattatgcaaaaagcaccctatacctgcaaatgaacagtctgaggtctgaggacacggccacttattactgtacaagagctggctatagcagctatcccgactactttgattactggggccaaggagtcatgctcacagtctcctcagctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 180 - protein, 470 aa, synthetic construct
  REGION 1..470: note = Synthetic Construct
MDIRLSLVFLVLFIKGVQCEVQLVESGGGLVQPGRSLKLSCVASGFTFNNYWMTWIRQAPGKGLEWVASITNTGGSTAYPDSVKGRFTISRDYAKSTLYLQMNSLRSEDTATYYCTRAGYSSYPDYFDYWGQGVMLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 181 - DNA, 702 bp, synthetic construct
  misc_feature 1..702: note = Light chain of clone 1361-5
  source 1..702: mol_type = other DNA
  CDS 1..702
atgatggctccagttcaactcttagggctgctgctgctctggctcccagccatgagatgtaacatccagatgacccagtctccttcactactctctgcatctgtgggagacagagtcactctcagctgcaaagcaggtcagaatatttacaattacttagcctggtatcagcaaaagcttggagaagctcccaaactcctgatttataatgcaaacagtttgcaaacgggcatcccatcaaggttcagtggcagtggatctggtacagatttcacactcaccatcagcagcctgcagcctgaagatgttgccacatatttctgccagcagtatagcagtgggtggacgttcggtggaggcaccaagctggaattgaaacgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttag

SEQ ID NO: 182 - protein, 233 aa, synthetic construct
  REGION 1..233: note = Synthetic Construct
MMAPVQLLGLLLLWLPAMRCNIQMTQSPSLLSASVGDRVTLSCKAGQNIYNYLAWYQQKLGEAPKLLIYNANSLQTGIPSRFSGSGSGTDFTLTISSLQPEDVATYFCQQYSSGWTFGGGTKLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 183 - DNA, 27 bp, synthetic construct
  misc_feature 1..27: note = 1361-5 forward primer
  source 1..27: mol_type = other DNA
agcactgcctatccagactctgtgaag

SEQ ID NO: 184 - DNA, 27 bp, synthetic construct
  misc_feature 1..27: note = 1361-5 reverse primer
  source 1..27: mol_type = other DNA
tggataggcagtgctaccaccagtatt

SEQ ID NO: 185 - DNA, 27 bp, synthetic construct
  misc_feature 1..27: note = 1361-7 forward primer
  source 1..27: mol_type = other DNA
ccagacgctgtgaagggccgattcact

SEQ ID NO: 186 - DNA, 27 bp, synthetic construct
  misc_feature 1..27: note = 1361-7 Reverse primer
  source 1..27: mol_type = other DNA
cttcacagcgtctggatagtaagtgct

SEQ ID NO: 187 - DNA, 27 bp, synthetic construct
  misc_feature 1..27: note = Fc_S239D_Fw
  source 1..27: mol_type = other DNA
ggaccggacgtcttcctcttcccccca

SEQ ID NO: 188 - DNA, 27 bp, synthetic construct
  misc_feature 1..27: note = Fc_S239D_Rv
  source 1..27: mol_type = other DNA
gaagacgtccggtccccccaggagttc

SEQ ID NO: 189 - DNA, 27 bp, synthetic construct
  misc_feature 1..27: note = Fc_A330L_I332E_Fw
  source 1..27: mol_type = other DNA
ccactgcccgaggagaaaaccatctcc

SEQ ID NO: 190 - DNA, 27 bp, synthetic construct
  misc_feature 1..27: note = Fc_A330L_I332E_Rv
  source 1..27: mol_type = other DNA
ctcctcgggcagtgggagggctttgtt

SEQ ID NO: 191 - DNA, 27 bp, synthetic construct
  misc_feature 1..27: note = Fc_I332E_E333A_Fw
  source 1..27: mol_type = other DNA
cccgaggccaaaaccatctccaaagcc

SEQ ID NO: 192 - DNA, 27 bp, synthetic construct
  misc_feature 1..27: note = Fc_I332E_E333A_Rv
  source 1..27: mol_type = other DNA
ggttttggcctcgggggctgggagggc

(19) United States
(12) Patent Application Publication
(10) Pub. No.: US 2024/0148893 A1
(43) Pub. Date: May 9, 2024

(54) ANTI-TMEM-180 ANTIBODY, ANTICANCER AGENT, AND TEST METHOD FOR CANCER

(71) Applicants: NATIONAL CANCER CENTER JAPAN, Tokyo (JP); RIN INSTITUTE INC., Tokyo (JP)

(72) Inventors: Yasuhiro MATSUMURA, Chiba (JP); Masahiro Yasunaga, Chiba (JP); Shinji Saijou, Chiba (JP); Shingo Hanaoka, Chiba (JP); Takahiro Anzai, Chiba (JP); Shino Manabe, Chiba (JP)

(21) Appl. No.: 18/178,854

(22) Filed: Mar. 6, 2023

Related U.S. Application Data
(63) Continuation of application No. 16/083,077, filed on Sep. 7, 2018, now Pat. No. 11,633,499.

(30) Foreign Application Priority Data
Mar. 8, 2016 (JP) 2016 - 044946
Aug. 22, 2016 (JP) 2016 - 161697

(51) Int. Cl. A61K 47/68; A61K 39/395; A61K 49/00; A61P 35/00; C07K 16/30; C12N 5/10; C12N 15/09; C12N 15/62; G01N 33/574

(52) U.S. Cl. CPC A61K 47/6851; A61K 39/395; A61K 49/00; A61P 35/00; C07K 16/30; C12N 5/10; C12N 15/09; C12N 15/62; G01N 33/574; G01N 33/57419

(57) ABSTRACT

An object of the present invention is to provide an anticancer drug capable of treating cancer by finding a target molecule specifically expressed in cancer cells and by specifically acting on the target molecule, and to provide a cancer testing method including a step of measuring the target molecule in a sample. The present invention provides an anticancer drug containing, as an active ingredient thereof, an anti-transmembrane protein 180 (TMEM-180) antibody or an antigen-binding fragment thereof. In addition, the present invention provides a cancer testing method including a step of measuring the amount of TMEM-180 in a sample collected from a subject.

### TECHNICAL FIELD

The present invention relates to a novel anti-TMEM-180 antibody, an anticancer drug containing an anti-TMEM-180 antibody, a cancer testing method involving measuring TMEM-180 in a sample, and the like.

### BACKGROUND ART

Recently, numerous molecular targeted drugs that act specifically on a specific molecule have been developed for use as anticancer drugs. In particular, development has been proceeding on various antibody drugs having, as antigens, molecules specifically expressed in certain cancer cells or molecules exhibiting increased expression in cancer cells. In the development of such antibody drugs, a comparison is first made between expression of mRNA in cancer tissue collected at the time of surgery and expression of mRNA in normal tissue collected from a nearby site, molecules specifically expressed only in cancer tissue or molecules exhibiting increased expression in cancer tissue are identified, and antibodies are then prepared by using those molecules as antigens.

Colon cancer is manifest from cells of the intestinal mucosa. Cetuximab has previously been developed for use against colon cancer as an antibody drug that targets epidermal growth factor receptor (EGFR) (Non-Patent Documents 1 to 3). However, since EGFR is also expressed in normal tissue, Cetuximab also has the potential to act on normal tissue, thereby resulting in the desire to develop a molecular targeted drug that targets molecules more specifically expressed during colon cancer. With regard to this point, since there is only a small amount of mucosal tissue in which colon cancer occurs, there has been the problem of it being difficult to identify a target molecule by comparing cancerous mucosal cells and normal mucosal cells.

### CITATION LIST

### Non-Patent Documents

Non-Patent Document 1: Cunningham, D., et al., The New England Journal of Medicine, Vol. 351, No. 4, 2004, pp. 337-345
Non-Patent Document 2: Goldstein, N. I., et al., Clin. Cancer Res., Vol. 1, 1311-1318, 1995
Non-Patent Document 3: Karapetis, C. S., et al., The New Engl. J. Med., Vol. 359, 1757-1765

### SUMMARY

### Technical Problem

An object of the present invention is to provide an anticancer drug capable of treating cancer by finding a target molecule specifically expressed in cancer cells and by specifically acting on the target molecule, and to provide a cancer testing method comprising a step of measuring the target molecule in a sample.

### Solution to Problem

In order to solve the above-mentioned problems, the inventors of the present invention compared mRNA expression between various types of colon cancer cell lines and normal mucosal cells contained in the washing liquid obtained during colonoscopy by analyzing using a DNA microarray to seek out molecules that are only expressed in colon cancer cell lines. As a result, transmembrane protein 180 (TMEM-180) was identified as a protein that is expressed in all colon cancer cell lines but which is not observed to be expressed in the cells of healthy subjects. Moreover, in addition to confirming that TMEM-180 is not expressed in normal colon tissue by quantitative PCR and in situ hybridization, TMEM-180 was also confirmed to not be expressed in prominent normal tissues, thereby confirming TMEM-180 to be an ideal molecule for use as a target of anticancer drugs and as an indicator in cancer testing.

Antibody to TMEM-180 was produced, and this antibody was confirmed to demonstrate a cytocidal effect on colon cancer cells as well as be able to detect recurrence in particular with greater sensitivity than conventional colon cancer markers, thereby leading to completion of the present invention.

Namely, the present invention is as indicated below.

[1] An anticancer drug containing, as an active ingredient thereof, an anti-transmembrane protein 180 (TMEM-180) antibody or an antigen-binding fragment thereof.

[2] An anticancer drug containing, as an active ingredient thereof, an anti-TMEM-180 antibody having a substance having anticancer activity bound thereto, or an antigen-binding fragment thereof.

[3] The anticancer drug described in [1] or [2] above, wherein the anti-TMEM-180 antibody has at least one of the six CDRs indicated below:

heavy chain CDR1:
(SEQ ID NO: 1)
GFSLTRYNVH;
heavy chain CDR2:
(SEQ ID NO: 2)
VIWTGGSTD;
heavy chain CDR3:
(SEQ ID NO: 3)
DLGY;
light chain CDR1:
(SEQ ID NO: 4)
KSSQSLKYRDGKTYLN;
light chain CDR2:
(SEQ ID NO: 5)
QVSKLDS;
and
light chain CDR3:
(SEQ ID NO: 6)
CQGSYSPHT.

[4] The anticancer drug described in [1] or [2] above, wherein the anti-TMEM-180 antibody has at least one of the six CDRs indicated below:

heavy chain CDR1:
(SEQ ID NO: 7)
GFSLTSYYMQ;
heavy chain CDR2:
(SEQ ID NO: 8)
FIRSGGSTE;
heavy chain CDR3:
(SEQ ID NO: 9)
AFYGGYYFDY;
light chain CDR1:
(SEQ ID NO: 10)
KASQNVGSNVD;
light chain CDR2:
(SEQ ID NO: 11)
KASNRYT;
and
light chain CDR3:
(SEQ ID NO: 12)
MQSNTKYT.

[5] The anticancer drug described in [1] or [2] above, wherein the anti-TMEM-180 antibody has at least one of the six CDRs indicated below:

heavy chain CDR1:
(SEQ ID NO: 40)
GFTFSDYAMA;
heavy chain CDR2:
(SEQ ID NO: 41)
TIIYDGSST;
heavy chain CDR3:
(SEQ ID NO: 42)
HWYWYFDF;
light chain CDR1:
(SEQ ID NO: 43)
LASEGISNDLA;
light chain CDR2:
(SEQ ID NO: 44)
AASRLQD;
and
light chain CDR3:
(SEQ ID NO: 45)
QQSYKYPLT.

[6] The anticancer drug described in [1] or [2] above, wherein the anti-TMEM-180 antibody has at least one of the six CDRs indicated below:

heavy chain CDR1:
(SEQ ID NO: 48)
DCALN;
heavy chain CDR2:
(SEQ ID NO: 49)
WINTQTGKPTYADDF;
heavy chain CDR3:
(SEQ ID NO: 50)
EDYGYFDY;
light chain CDR1:
(SEQ ID NO: 51)
QASQNINKFIA;
light chain CDR2:
(SEQ ID NO: 52)
YTSTLVS;
and
light chain CDR3:
(SEQ ID NO: 53)
LQYDNLRT.

[7] The anticancer drug described in [1] or [2] above, wherein the anti-TMEM-180 antibody has at least one of the six CDRs indicated below:

heavy chain CDR1:
(SEQ ID NO: 56)
NYGMH;
heavy chain CDR2:
(SEQ ID NO: 57)
SISPSGGSTYYRDSV;
heavy chain CDR3:
(SEQ ID NO: 58)
SASITAYYYVMDA;
light chain CDR1:
(SEQ ID NO: 59)
KASQNVGSNVD;
light chain CDR2:
(SEQ ID NO: 60)
KASNRYT;
and
light chain CDR3:
(SEQ ID NO: 61)
MQSNSYPPT.

[8] The anticancer drug described in [1] or [2] above, wherein the anti-TMEM-180 antibody has at least one of the six CDRs indicated below:

heavy chain CDR1:
(SEQ ID NO: 64)
NYWMT;
heavy chain CDR2:
(SEQ ID NO: 65)
SITNTGGSTYYPDSV;
heavy chain CDR3:
(SEQ ID NO: 66)
AGYSSYPDYFDY;
light chain CDR1:
(SEQ ID NO: 67)
KAGQNIYNYLA;
light chain CDR2:
(SEQ ID NO: 68)
NANSLQT;
and
light chain CDR3:
(SEQ ID NO: 69)
QQYSSGWT.

[9] The anticancer drug described in [1] or [2] above, wherein the anti-TMEM-180 antibody has at least one of the six CDRs indicated below:

heavy chain CDR1:
(SEQ ID NO: 72)
DYWVS;
heavy chain CDR2:
(SEQ ID NO: 73)
EIYPNSGATNFNENFK;
heavy chain CDR3:
(SEQ ID NO: 74)
DGTMGIAYYFDY;
light chain CDR1:
(SEQ ID NO: 75)
KASQNINRYLN;
light chain CDR2:
(SEQ ID NO: 76)
NANSLQT;
and
light chain CDR3:
(SEQ ID NO: 77)
LQHNSWPYT.

[10] The anticancer drug described in [1] or [2] above, wherein the anti-TMEM-180 antibody has at least one of the six CDRs indicated below:

heavy chain CDR1:
(SEQ ID NO: 80)
SYDIS;
heavy chain CDR2:
(SEQ ID NO: 81)
AINPGSGGTGYNEKFKGK;
heavy chain CDR3:
(SEQ ID NO: 82)
IHGGYRYWFAY;
light chain CDR1:
(SEQ ID NO: 83)
RASSSVSYMH;
light chain CDR2:
(SEQ ID NO: 84)
DTSKLAS;
and
light chain CDR3:
(SEQ ID NO: 85)
LQRSSYPPT.

[11] The anticancer drug described in [1] or [2] above, wherein the anti-TMEM-180 antibody has at least one of the six CDRs indicated below:

heavy chain CDR1:
(SEQ ID NO: 88)
SNGVG;
heavy chain CDR2:
(SEQ ID NO: 89)
TIWTGGGTNYNSGVQS;
heavy chain CDR3:
(SEQ ID NO: 90)
EYMGFDY;
light chain CDR1:
(SEQ ID NO: 91)
KASQNVGINVG;
light chain CDR2:
(SEQ ID NO: 92)
WASNRDT;
and
light chain CDR3:
(SEQ ID NO: 93)
LQHNSYPRT.

[12] The anticancer drug described in [1] or [2] above, wherein the anti-TMEM-180 antibody has at least one of the six CDRs indicated below:

heavy chain CDR1:
(SEQ ID NO: 96)
SNGVG;
heavy chain CDR2:
(SEQ ID NO: 97)
TIWSGGGTNYNSAVQS;
heavy chain CDR3:
(SEQ ID NO: 98)
EEKGFAY;
light chain CDR1:
(SEQ ID NO: 99)
KASQNVGINVG;
light chain CDR2:
(SEQ ID NO: 100)
WASNRDT;
and
light chain CDR3:
(SEQ ID NO: 101)
LQHNSYPRA.

[13] The anticancer drug described in any one of [3] to above, wherein the anti-TMEM-180 antibody is such that
at least one of the heavy chain CDR1 to CDR3 and light chain CDR1 to CDR3 contains one to several amino acid additions, substitutions or deletions, or
at least one of the heavy chain CDR1 to CDR3 and light chain CDR1 to CDR3 has an amino acid sequence having identity of 80% or more with the amino acid sequences of the heavy chain CDR1 to CDR3 and light chain CDR1 to CDR3.

[14] The anticancer drug described in [1] or [2] above, wherein the anti-TMEM-180 antibody contains:
a heavy chain containing the amino acid sequence represented by SEQ ID NO: 13;
a heavy chain containing an amino acid sequence containing one to several amino acid additions, substitutions or deletions in the amino acid sequence represented by SEQ ID NO: 13; or
a heavy chain containing an amino acid sequence having identity of 80% or more with the amino acid sequence represented by SEQ ID NO: 13, and/or
a light chain containing the amino acid sequence represented by SEQ ID NO: 14;
a light chain containing an amino acid sequence containing one to several amino acid additions, substitutions or deletions in the amino acid sequence represented by SEQ ID NO: 14; or
a light chain containing an amino acid sequence having identity of 80% or more with the amino acid sequence represented by SEQ ID NO: 14.

[15] The anticancer drug described in [1] or [2] above, wherein the anti-TMEM-180 antibody contains:
a heavy chain containing the amino acid sequence represented by SEQ ID NO: 15;
a heavy chain containing an amino acid sequence containing one to several amino acid additions, substitutions or deletions in the amino acid sequence represented by SEQ ID NO: 15; or
a heavy chain containing an amino acid sequence having identity of 80% or more with the amino acid sequence represented by SEQ ID NO: 15, and/or
a light chain containing the amino acid sequence represented by SEQ ID NO: 16;
a light chain containing an amino acid sequence containing one to several amino acid additions, substitutions or deletions in the amino acid sequence represented by SEQ ID NO: 16; or
a light chain containing an amino acid sequence having identity of 80% or more with the amino acid sequence represented by SEQ ID NO: 16.

[16] The anticancer drug described in [1] or [2] above, wherein the anti-TMEM-180 antibody contains:
a heavy chain containing the amino acid sequence represented by SEQ ID NO: 46;
a heavy chain containing an amino acid sequence containing one to several amino acid additions, substitutions or deletions in the amino acid sequence represented by SEQ ID NO: 46; or
a heavy chain containing an amino acid sequence having identity of 80% or more with the amino acid sequence represented by SEQ ID NO: 46, and/or
a light chain containing the amino acid sequence represented by SEQ ID NO: 47;
a light chain containing an amino acid sequence containing one to several amino acid additions, substitutions or deletions in the amino acid sequence represented by SEQ ID NO: 47; or
a light chain containing an amino acid sequence having identity of 80% or more with the amino acid sequence represented by SEQ ID NO: 47.

[17] The anticancer drug described in [1] or [2] above, wherein the anti-TMEM-180 antibody contains:
a heavy chain containing the amino acid sequence represented by SEQ ID NO: 54;
a heavy chain containing an amino acid sequence containing one to several amino acid additions, substitutions or deletions in the amino acid sequence represented by SEQ ID NO: 54; or
a heavy chain containing an amino acid sequence having identity of 80% or more with the amino acid sequence represented by SEQ ID NO: 54, and/or
a light chain containing the amino acid sequence represented by SEQ ID NO: 55;
a light chain containing an amino acid sequence containing one to several amino acid additions, substitutions or deletions in the amino acid sequence represented by SEQ ID NO: 55; or
a light chain containing an amino acid sequence having identity of 80% or more with the amino acid sequence represented by SEQ ID NO: 55.

[18] The anticancer drug described in [1] or [2], wherein the anti-TMEM-180 antibody contains:
a heavy chain containing the amino acid sequence represented by SEQ ID NO: 62;
a heavy chain containing an amino acid sequence containing one to several amino acid additions, substitutions or deletions in the amino acid sequence represented by SEQ ID NO: 62; or
a heavy chain containing an amino acid sequence having identity of 80% or more with the amino acid sequence represented by SEQ ID NO: 62, and/or
a light chain containing the amino acid sequence represented by SEQ ID NO: 63;
a light chain containing an amino acid sequence containing one to several amino acid additions, substitutions or deletions in the amino acid sequence represented by SEQ ID NO: 63; or
a light chain containing an amino acid sequence having identity of 80% or more with the amino acid sequence represented by SEQ ID NO: 63.

[19] The anticancer drug described in [1] or [2] above, wherein the anti-TMEM-180 antibody contains:
a heavy chain containing the amino acid sequence represented by SEQ ID NO: 70;
a heavy chain containing an amino acid sequence containing one to several amino acid additions, substitutions or deletions in the amino acid sequence represented by SEQ ID NO: 70; or
a heavy chain containing an amino acid sequence having identity of 80% or more with the amino acid sequence represented by SEQ ID NO: 70, and/or
a light chain containing the amino acid sequence represented by SEQ ID NO: 71;
a light chain containing an amino acid sequence containing one to several amino acid additions, substitutions or deletions in the amino acid sequence represented by SEQ ID NO: 71; or
a light chain containing an amino acid sequence having identity of 80% or more with the amino acid sequence represented by SEQ ID NO: 71.

[20] The anticancer drug described in [1] or [2] above, wherein the anti-TMEM-180 antibody contains:
a heavy chain containing the amino acid sequence represented by SEQ ID NO: 78;
a heavy chain containing an amino acid sequence containing one to several amino acid additions, substitutions or deletions in the amino acid sequence represented by SEQ ID NO: 78; or
a heavy chain containing an amino acid sequence having identity of 80% or more with the amino acid sequence represented by SEQ ID NO: 78, and/or
a light chain containing the amino acid sequence represented by SEQ ID NO: 79;
a light chain containing an amino acid sequence containing one to several amino acid additions, substitutions or deletions in the amino acid sequence represented by SEQ ID NO: 79; or
a light chain containing an amino acid sequence having identity of 80% or more with the amino acid sequence represented by SEQ ID NO: 79.

[21] The anticancer drug described in [1] or [2] above, wherein the anti-TMEM-180 antibody contains:
a heavy chain containing the amino acid sequence represented by SEQ ID NO: 86;
a heavy chain containing an amino acid sequence containing one to several amino acid additions, substitutions or deletions in the amino acid sequence represented by SEQ ID NO: 86; or
a heavy chain containing an amino acid sequence having identity of 80% or more with the amino acid sequence represented by SEQ ID NO: 86, and/or
a light chain containing the amino acid sequence represented by SEQ ID NO: 87;
a light chain containing an amino acid sequence containing one to several amino acid additions, substitutions or deletions in the amino acid sequence represented by SEQ ID NO: 87; or
a light chain containing an amino acid sequence having identity of 80% or more with the amino acid sequence represented by SEQ ID NO: 87.

[22] The anticancer drug described in [1] or [2] above, wherein the anti-TMEM-180 antibody contains:
a heavy chain containing the amino acid sequence represented by SEQ ID NO: 94;
a heavy chain containing an amino acid sequence containing one to several amino acid additions, substitutions or deletions in the amino acid sequence represented by SEQ ID NO: 94; or
a heavy chain containing an amino acid sequence having identity of 80% or more with the amino acid sequence represented by SEQ ID NO: 94, and/or
a light chain containing the amino acid sequence represented by SEQ ID NO: 95;
a light chain containing an amino acid sequence containing one to several amino acid additions, substitutions or deletions in the amino acid sequence represented by SEQ ID NO: 95; or
a light chain containing an amino acid sequence having identity of 80% or more with the amino acid sequence represented by SEQ ID NO: 95.

[23] The anticancer drug described in [1] or [2] above, wherein the anti-TMEM-180 antibody contains:
a heavy chain containing the amino acid sequence represented by SEQ ID NO: 102;
a heavy chain containing an amino acid sequence containing one to several amino acid additions, substitutions or deletions in the amino acid sequence represented by SEQ ID NO: 102; or
a heavy chain containing an amino acid sequence having identity of 80% or more with the amino acid sequence represented by SEQ ID NO: 102, and/or
a light chain containing the amino acid sequence represented by SEQ ID NO: 103;
a light chain containing an amino acid sequence containing one to several amino acid additions, substitutions or deletions in the amino acid sequence represented by SEQ ID NO: 103; or
a light chain containing an amino acid sequence having identity of 80% or more with the amino acid sequence represented by SEQ ID NO: 103.

[24] The anticancer drug described in any one of [1] to above, wherein the anti-TMEM-180 antibody has one or more N-linked oligosaccharides bound to the Fc region and fucose is not bound to N-acetylglucosamine on the reducing end of the N-linked oligosaccharide.

[25] The anticancer drug described in any one of [1] to above, which is targeted at cancer expressing TMEM-180.

[26] The anticancer drug described in any one of [1] to above, which is targeted at colon cancer.

[27] The anticancer drug described in any one of [1] to above, which is administered concomitantly with vaccine therapy using a composition containing at least one peptide comprising an amino acid sequence represented by SEQ ID NOs: 104 to 170.

[28] An anti-TMEM-180 antibody described in any one of [3] to above or an antigen-binding fragment thereof.

[29] A nucleic acid encoding any one of the heavy chains CDR1 to CDR3 and light chains CDR1 to CDR3 described in any one of [3] to above.

[30] A nucleic acid encoding any one of the heavy chains and light chains described in any one of to above.

[31] An expression vector containing the nucleic acid described in or above.

[32] A transformant containing the expression vector described in above.

[33] A method for producing an anti-TMEM-180 antibody, or an antigen-binding fragment thereof, the method comprising the steps of:
expressing an antibody in the transformant described in above; and
recovering the antibody.

[34] A cancer testing method, comprising a step of measuring the amount of TMEM-180 in a sample collected from a subject.

[35] The cancer testing method described in above, wherein the amount of TMEM-180 is measured by immunoassay using an anti-TMEM-180 antibody or an antigen-binding fragment thereof.

[36] The cancer testing method described in above, wherein the anti-TMEM-180 antibody or antigen-binding fragment thereof is the anti-TMEM-180 antibody or antigen-binding fragment thereof described in above.

[37] The cancer testing method described in any one of to above, which is targeted at colon cancer.

[38] The cancer testing method described in to [37], which is used to test for recurrence or metastasis following treatment.

[39] A cancer testing kit containing an anti-TMEM-180 antibody or an antigen-binding fragment thereof.

[40] The cancer testing kit described in above, wherein the anti-TMEM-180 antibody or antigen-binding fragment thereof is the anti-TMEM-180 antibody or antigen-binding fragment thereof described in above.

[41] The cancer testing kit described in or above, which is targeted at colon cancer.

### Advantageous Effects of Invention

The present inventors also discovered that TMET-180 is existed and presented on the surface of the exosomes in the serum from a subject having cancer. According to this finding, the invention below is also provided.

[A17] A cancer testing method, comprising a step of detecting the amount of exosomes having TMEM-180 in a body fluid sample obtained from a subject.

[A18] The cancer testing method according to [A17], wherein the step of detecting the amount of exosomes having TMEM-180 is conducted with an antibody binding to TMEM-180 or an antigen-binding fragment thereof.

[A21] The cancer testing method according to [A18], wherein the step of detecting the amount of exosomes having TMEM-180 is conducted with the anti-TMEM-180 antibody according to any one of [3] to or an antigen-binding fragment thereof.

[A22] A pharmaceutical composition for use in treating cancer in a subject having cancer, comprising an antibody binding to TMEM-180, wherein the subject is a subject whose body fluid sample is a sample from which TMEM-180 has been detected.

The present inventors further obtained a 1361-5 clone antibody, in which non-specific attachment has been reduced, from a 1361 antibody. Thus, the present invention also provides the following inventions.

[A1] An antibody binding to the transmembrane protein 180 (TMEM-180) or an antigen-binding fragment thereof,
(1) wherein the antibody comprises a heavy chain variable region having
a heavy chain CDR1: NYWMT (SEQ ID NO: 171);
a heavy chain CDR2: SITNTGGSTAYPDSV (SEQ ID NO: 172); and
a heavy chain CDR3: AGYSSYPDYFDY (SEQ ID NO: 173), and
a light chain variable region having
a light chain CDR1: KAGQNIYNYLA (SEQ ID NO: 174);
a light chain CDR2: NANSLQT (SEQ ID NO: 175); and
a light chain CDR3: QQYSSGWT (SEQ ID NO: 176); or
(2) wherein the antibody competes with the antibody according to above (1) for binding to TMEM-180.

[A2] The antibody according to above [A1] or an antigen-binding fragment thereof, wherein the antibody comprises a heavy chain variable region (SEQ ID NO: 177) and a light chain variable region (SEQ ID NO: 178).

[A3] The antibody according to above [A1] or [A2] or an antigen-binding fragment thereof, wherein the antibody has a heavy chain (SEQ ID NO: 180) and a light chain (SEQ ID NO: 182).

[A4] The antibody according to any one of above [A1] to [A3], wherein the antibody has one or more N-linked oligosaccharides bound to the Fc region and fucose is not bound to N-acetylglucosamine on the reducing end of the N-linked oligosaccharide.

[A5] A pharmaceutical composition for use in treating cancer, comprising the antibody according to any one of above [A1] to [A4].

[A6] A composition for use in diagnosing cancer, comprising the antibody according to any one of above [A1] to [A4] or an antigen-binding fragment thereof.

[A7] A diagnostic kit for use in diagnosing cancer, comprising the antibody according to any one of above [A1] to [A4] or an antigen-binding fragment thereof.

[A8] The pharmaceutical composition according to above [A5], which is targeted at cancer expressing TMEM-180.

[A9] The pharmaceutical composition according to above [A5], which is targeted at colon cancer.

[A10] A nucleic acid encoding any one of the heavy chain CDRs 1 to 3 and the light chain CDRs 1 to 3 as defined in any one of above [A1] to [A4].

[A11] A nucleic acid encoding any one of the heavy chain and the light chain according to any one of above [A1] to [A4].

[A12] A vector comprising the nucleic acid according to above [A10] or [A11].

[A13] A transformant comprising the vector according to above [A12].

[A14] A cancer testing method comprising a step of measuring the amount of TMEM-180 in the sample obtained from a subject with the antibody according to above [A1] to [A4] or an antigen-binding fragment thereof.

[A15] The cancer testing method according to above [A14], which is targeted at colon cancer.

[A16] The cancer testing method according to above [A14] or [A15], which is used for testing recurrence or metastasis after a treatment.

[A19] The method according to above [A18], wherein the step of detecting the amount of exosomes having TMEM-180 is conducted with the antibody according to any one of [A1] to [A4] or an antigen-binding fragment thereof.

[A20] A method for detecting stage 1 or stage 2 cancer, comprising a step of measuring the amount of TMEM-180 in the sample obtained from a subject.

[A23] The cancer testing method according to any one of above [A17] to [A19], for use in detecting stage 1 or stage 2 cancer.

[A24] The method according to any one of above [A17] to [A19], wherein the cancer is colon cancer.

[B1] A pharmaceutical composition for use in treating cancer, comprising an antibody-drug conjugate of the anti-TMEM-180 and a cytotoxic agent.

[B2] The pharmaceutical composition according to above [B1],
wherein the anti-TMEM-180 antibody is
(1) an antibody comprising
a heavy chain having
a heavy chain CDR1: NYWMT (SEQ ID NO: 171);
a heavy chain CDR2: SITNTGGSTAYPDSV (SEQ ID NO: 172); and
a heavy chain CDR3: AGYSSYPDYFDY (SEQ ID NO: 173); and
a light chain having
a light chain CDR1: KAGQNIYNYLA (SEQ ID NO: 174);
a light chain CDR2: NANSLQT (SEQ ID NO: 175); and
a light chain CDR3: QQYSSGWT (SEQ ID NO: 176); or
(2) an antibody which competes with the antibody according to above (1) for binding to TMEM-180.

[B3] The pharmaceutical composition according to above [B2], wherein the anti-TMEM-180 antibody has a heavy chain (SEQ ID NO: 180) and a light chain (SEQ ID NO: 182).

[B4] The pharmaceutical composition according to above [B1],
wherein the anti-TMEM-180 antibody is
(1) an antibody comprising
a heavy chain comprising
a heavy chain CDR1: DYWVS (SEQ ID NO: 72);
a heavy chain CDR2: EIYPNSGATNFNENFK (SEQ ID NO: 73); and
a heavy chain CDR3: DGTMGIAYYFDY (SEQ ID NO: 74); and
a light chain comprising
a light chain CDR1: KASQNINRYLN (SEQ ID NO: 75);
a light chain CDR2: NANSLQT (SEQ ID NO: 76); and
a light chain CDR3: LQHNSWPYT (SEQ ID NO: 77); or
(2) an antibody which competes with the antibody according to above (1) for binding to TMEM-180.

[B5] The pharmaceutical composition according to any one of above [B1] to [B4], wherein the antibody and the drug are linked via a cleavable linker.

[B6] The pharmaceutical composition according to above [B5], wherein the cleavable linker is a linker which is cleavable with peptidase in bodies, a hydrazine linker, or a linker comprising a carbamate bond or an ester bond.

[B7] The pharmaceutical composition according to above [B6], wherein the cleavable linker is a linker comprising a dipeptide of valine-citrulline or phenylalanine-lysin.

[B8] The pharmaceutical composition according to above [B7], wherein the cleavable linker is a valine-citrulline linker.

[B9] The pharmaceutical composition according to above [B8], wherein the cleavable linker comprises a polyethylene glycol block and a valine-citrulline block (for example, comprises Mal-PEG₁₂-Val-Cit-PABC).

[B10] The pharmaceutical composition according to any one of above [B1] to [B9], in particular [B9], wherein the cytotoxic agent is monomethylauristatin E.

[B11] The pharmaceutical composition according to above [B9] or [B10],

wherein the antibody is the antibody according to any one of above [1] to and [A1].

[B12] A pharmaceutical composition comprising an antibody-drug conjugate of anti-TMEM-180 antibody and a cytotoxic agent; or a pharmaceutical composition for use in treating cancer according to above [A5], wherein the anti-TMEM-180 antibody is in a form linked to a cytotoxic agent.

The anticancer drug containing an anti-TMEM-180 antibody or an antigen-binding fragment thereof according to the present invention targets TMEM-180, which is a protein specifically expressed in certain cancers but is not expressed in normal tissue, and is thought to allow the obtaining of a potent effect specific to cancer cells with few adverse side effects.

Since TMEM-180 is specifically expressed in certain cancers but not expressed in normal tissue, a cancer testing method and testing kit that uses the anti-TMEM-180 antibody or antigen-binding fragment thereof according to the present invention enables cancer testing to be carried out easily. The cancer testing method and testing kit according to the present invention are able to detect recurrence of cancer in particular with greater sensitivity in comparison with conventional colon cancer markers.

In addition, according to the anticancer drug containing an anti-TMEM-180 antibody having a substance having anticancer activity bound thereto, or an antigen-binding fragment thereof, according to the present invention, that anticancer drug can be specifically delivered to cancer cells expressing TMEM-180, thereby facilitating the use of a highly toxic substance for the substance having anticancer activity.

### BRIEF DESCRIPTION OF THE DRAWINGS

FIG. 1A indicates expression levels of TMEM-180 obtained as a result of conducting an exhaustive expression analysis on human colon cancer cell lines and colon mucosal cells obtained from healthy subjects by DNA microarray analysis.

FIG. 1B indicates the results of analyzing expression of TMEM-180 in colon cancer cells and adjacent normal colon tissue by quantitative PCR. FIGS. 1C and 1D indicate the results of investigating expression of TMEM-180 at cancerous sites and normal sites by in situ hybridization.

FIG. 2A indicates the results of investigating expression of TMEM-180 in various normal tissues using a database. FIG. 2B indicates the results of investigating expression of trace amounts of TMEM-180 for molecules targeted by TMEM-180 and conventional anticancer drugs using the same method as in FIG. 2A.

FIG. 3 indicates the results of a flow cytometry analysis of various colon cancer, brain tumor and hematologic cancer cell lines using anti-TMEM-180 antibody clones 98, 101 and 212.

FIG. 4 indicates the results of immunofluorescent staining of colon cancer cells by anti-TMEM-180 antibody clones 98, 101 and 212. Furthermore, the antibody concentrations of each clone are not aligned.

FIG. 5 indicates the results of investigating the reactivity of rat IgM antibody clone 98 and rat-human chimeric antibody clone 98 with colon cancer cell line DLD-1 and a TMEM-180 knockout line thereof by flow cytometry.

FIG. 6 indicates the results of staining human colon cancer and nearby normal tissue using rat IgM antibody clone 98.

FIG. 7 indicates the results of a cytotoxicity test using culture supernatant of a hybridoma producing rat IgM antibody clone 98. Antibody concentration in the hybridoma culture supernatant was measured by ELISA using IgM-specific antibody.

FIG. 8 indicates the results of a cytotoxicity test using culture supernatant of a hybridoma producing rat IgM antibody clone 101. Antibody concentration in the hybridoma culture supernatant was measured by ELISA using IgM-specific antibody.

FIG. 9 indicates the results of measuring TMEM-180 protein concentration in culture supernatants of cancer cells.

FIG. 10 indicates the results of measuring plasma TMEM-180 protein concentrations in four stage IV colon cancer patients. In addition, the table shown to the right of the graph indicates the results of measuring CEA levels in specimens collected from the same patients.

FIG. 11 indicates the results of measuring plasma TMEM-180 protein concentrations before and after surgery in three stage III colon cancer patients.

FIG. 12 indicates the results of measuring plasma TMEM-180 protein concentrations and CEA concentrations before surgery, after surgery and at the time of recurrence in colon cancer patients at various stages.

FIG. 13 shows that the 1361-5 antibody does not recognize normal skin tissues.

FIG. 14 shows that the 1361-5 antibody does not recognize normal tissues besides skin.

FIG. 15 shows that the 1361-5 antibody binds to the surface of a colon cancer cell line DLD-1.

FIG. 16 shows that the 1361-5 antibody binds to the surface of a colon cancer cell line DLD-1.

FIG. 17 shows that the 1361-5 antibody well stains the tissues on a colon cancer tissue array.

FIG. 18 shows that the exosomes existing in the culture supernatant of a colon cancer cell line DLD-1 contains TMEM-180 protein. In the figure, an immobilized antibody means the antibody which is immobilized on the plate surface. The detection of the exosomes was performed with an anti-CD9 antibody.

FIG. 19 shows the amount of 98 antibody positive components in the fraction obtained by a gel-filtration of the culture supernatant of a TMEM-180-forcibly expressed line of a colon cancer cell line DLD-1.

FIG. 20A shows the results of evaluating the ADCC activity of cetuximab on a colon cancer cell line DLD-1.

FIG. 20B shows the results of evaluating the ADCC activity of the 1361-5 antibody on a colon cancer cell line DLD-1.

FIG. 20C shows the results of evaluating the ADCC activity of a Fc variant of the 1361-5 antibody (Variant 1) on a colon cancer cell line DLD-1.

FIG. 20D shows the results of evaluating the ADCC activity of a Fc variant of the 1361-5 antibody (Variant 2) on a colon cancer cell line DLD-1.

FIG. 21 shows that a humanized 669 IgG antibody has a strong anti-cancer effect in mouse models transplanted with human colon cancer cells. In the figure, “Erb” indicates the results in the Erbitux-administrated group.

FIG. 22 shows that the exosomes contained in serums from colon cancer patients (51 cases from stage 1 to stage 4) are TMEM-180 positive.

FIG. 23 shows the electron microscope image showing that TMEM-180 is expressed on the surface of the exosomes.

FIG. 24 is a diagram showing the antibody-drug conjugate (ADC) prepared in Example C15.

FIG. 25 is a diagram showing internalization of the TMEM-180 antibody.

FIG. 26 shows a diagram showing cytotoxicity of the ADC prepared in Example 15C.

FIG. 27 shows positions of the guide RNAs used in preparing TMEM-180 knockout mice and disrupted sequences of the gene.

### DESCRIPTION OF EMBODIMENTS

(Anti-TMEM-180 Antibody and Anticancer Drug)

One aspect of the anticancer drug according to the present invention contains an anti-TMEM-180 antibody, or an antigen-binding fragment thereof, as active ingredient.

In the present description, the anti-TMEM-180 antibody refers to an antibody that binds to the transmembrane protein 180 (TMEM-180), and the antibody may specifically bind to TMEM-180. In the present description, in the case of referring to TMEM-180, the TMEM-180 may be TMEM-180 derived from any animal, and may be a mutant thereof provided it serves as a target of an anticancer drug or an indicator of a cancer testing method. The amino acid sequence of human TMEM-180 is indicated in SEQ ID NO: 17 as an example of TMEM-180.

In the present description, an “antibody” refers to an immunoglobulin, that is, a protein having a structure in which two heavy chains (H chains) and two light chains (L chains) are associated as stabilized by a pair of disulfide bonds. The heavy chains are formed from a heavy chain variable region VH, heavy chain constant regions CH1, CH2 and CH3, and a hinge region between CH1 and CH2, while the light chains are formed from a light chain variable region VL and a light chain constant region CL. Among these chains, a variable region fragment (Fv) formed of VH and VL is directly involved in antigen binding and is a region that provides the antibody with diversity. In addition, an antigen-binding region formed of VL, CL, VH and CH1 is referred to as the Fab region, while the region formed of the hinge region, CH2 and CH3 is referred to as the Fc region.

Among the variable regions, the region that makes direct contact with antigen undergoes considerable change in particular, and is referred to as a complementarity-determining region (CDR). The region other than CDR that remains comparatively unchanged is referred to as the framework region (FR). Three CDRs each are present in the variable regions of the light chain and heavy chain, and are referred to as heavy chain CDR1 to CDR3 and light chain CDR1 to CDR3, respectively, in order starting from the side of the N-terminal.

The term “treatment” as used herein means therapeutic treatment and prophylactic treatment. Therefore, the phrase “a pharmaceutical composition for use in treating cancer” means a pharmaceutical composition for use in therapeutically or prophylactically treating cancer, for example, an anti-cancer agent.

The anti-TMEM-180 antibody according to the present invention may be a monoclonal antibody or polyclonal antibody. In addition, the anti-TMEM-180 antibody of the present invention may be an isotype of any of IgG, IgM, IgA, IgD or IgE. It may also be that produced by immunizing a non-human animal such as a mouse, rat, hamster, guinea pig, rabbit or chicken, or may be a recombinant antibody, chimeric antibody, humanized antibody, fully human antibody or the like. A chimeric antibody refers to an antibody in which fragments of antibodies from different species are linked.

A “humanized antibody” refers to an antibody in which a location corresponding to human antibody has been substituted with an amino acid sequence specific to an antibody from a non-human origin, and an example thereof is an antibody that has the heavy chain CDR1 to CDR3 and light chain CDR1 to CDR3 of an antibody produced by immunizing a mouse or rat, and in which all other regions, including each of the four framework regions (FR) of the heavy chain and light chain, are derived from human antibody. This type of antibody may also be referred to as a CDR-grafted antibody. The term “humanized antibody” may also include human chimeric antibody.

The term “human chimera antibody” is an antibody derived from a non-human antibody, in which constant regions of the non-human antibody are replaced with constant regions of a human antibody. In a human chimera antibody, for example, IgG1 can be used as a subtype of the human antibody for the constant regions in light of obtaining a high ADCC activity.

In the present description, an “antigen-binding fragment” refers to a fragment of the antibody that binds to TMEM-180. More specifically, examples thereof include, but are not limited to, Fab formed of a VL, VH, CL and CH1 region, F(ab′)2 obtained by linking two Fab in a hinge region with disulfide bonds, Fv formed of VL and VH, single-stranded antibody in the form of scFv, in which VL and VH are linked with an artificial polypeptide linker, as well as bispecific antibodies such as diabodies, scDb, tandem sdFv and leucine zippers.

One aspect of the anti-TMEM-180 antibody according to the present invention has at least one of the six CDRs indicated below. These CDRs are the CDR sequences of anti-TMEM-180 antibody clone 98 indicated in the examples to be subsequently described.

Heavy chain CDR1:
(SEQ ID NO: 1)
GFSLTRYNVH
Heavy chain CDR2:
(SEQ ID NO: 2)
VIWTGGSTD
Heavy chain CDR3:
(SEQ ID NO: 3)
DLGY
Light chain CDR1:
(SEQ ID NO: 4)
KSSQSLKYRDGKTYLN
Light chain CDR2:
(SEQ ID NO: 5)
QVSKLDS
Light chain CDR3:
(SEQ ID NO: 6)
CQGSYSPHT

One aspect of the anti-TMEM-180 antibody according to the present invention has at least one of the six CDRs indicated below. These CDRs are the CDR sequences of anti-TMEM-180 antibody clone 101 indicated in the examples to be subsequently described.

Heavy chain CDR1:
(SEQ ID NO: 7)
GFSLTSYYMQ
Heavy chain CDR2:
(SEQ ID NO: 8)
FIRSGGSTE
Heavy chain CDR3:
(SEQ ID NO: 9)
AFYGGYYFDY
Light chain CDR1:
(SEQ ID NO: 10)
KASQNVGSNVD
Light chain CDR2:
(SEQ ID NO: 11)
KASNRYT
Light chain CDR3:
(SEQ ID NO: 12)
MQSNTKYT

One aspect of the anti-TMEM-180 antibody according to the present invention has at least one of the six CDRs indicated below. These CDRs are the CDR sequences of anti-TMEM-180 antibody clone 212 indicated in the examples to be subsequently described.

Heavy chain CDR1:
(SEQ ID NO: 40)
GFTFSDYAMA
Heavy chain CDR2:
(SEQ ID NO: 41)
TIIYDGSST
Heavy chain CDR3:
(SEQ ID NO: 42)
HWYWYFDF
Light chain CDR1:
(SEQ ID NO: 43)
LASEGISNDLA
Light chain CDR2:
(SEQ ID NO: 44)
AASRLQD
Light chain CDR3:
(SEQ ID NO: 45)
QQSYKYPLT

One aspect of the anti-TMEM-180 antibody according to the present invention has at least one of the six CDRs indicated below. These CDRs are the CDR sequences of anti-TMEM-180 antibody clone 129 indicated in the examples to be subsequently described.

Heavy chain CDR1:
(SEQ ID NO: 48)
DCALN
Heavy chain CDR2:
(SEQ ID NO: 49)
WINTQTGKPTYADDF
Heavy chain CDR3:
(SEQ ID NO: 50)
EDYGYFDY
Light chain CDR1:
(SEQ ID NO: 51)
QASQNINKFIA
Light chain CDR2:
(SEQ ID NO: 52)
YTSTLVS
Light chain CDR3:
(SEQ ID NO: 53)
LQYDNLRT

One aspect of the anti-TMEM-180 antibody according to the present invention has at least one of the six CDRs indicated below. These CDRs are the CDR sequences of anti-TMEM-180 antibody clone 382 indicated in the examples to be subsequently described.

Heavy chain CDR1:

(SEQ ID NO: 56)

NYGMH
Heavy chain CDR2:

(SEQ ID NO: 57)

SISPSGGSTYYRDSV
Heavy chain CDR3:

(SEQ ID NO: 58)

SASITAYYYVMDA
Light chain CDR1:

(SEQ ID NO: 59)

KASQNVGSNVD
Light chain CDR2:

(SEQ ID NO: 60)

KASNRYT
Light chain CDR3:

(SEQ ID NO: 61)

MQSNSYPPT

One aspect of the anti-TMEM-180 antibody according to the present invention has at least one of the six CDRs indicated below. These CDRs are the CDR sequences of anti-TMEM-180 antibody clone 1361 indicated in the examples to be subsequently described.

Heavy chain CDR1:

(SEQ ID NO: 64)

NYWMT
Heavy chain CDR2:

(SEQ ID NO: 65)

SITNTGGSTYYPDSV
Heavy chain CDR3:

(SEQ ID NO: 66)

AGYSSYPDYFDY
Light chain CDR1:

(SEQ ID NO: 67)

KAGQNIYNYLA
Light chain CDR2:

(SEQ ID NO: 68)

NANSLQT
Light chain CDR3:

(SEQ ID NO: 69)

QQYSSGWT

One aspect of the anti-TMEM-180 antibody according to the present invention has at least one of the six CDRs indicated below. These CDRs are the CDR sequences of anti-TMEM-180 antibody clone 669 indicated in the examples to be subsequently described.

Heavy chain CDR1:

(SEQ ID NO: 72)

DYWVS
Heavy chain CDR2:

(SEQ ID NO: 73)

EIYPNSGATNFNENFK
Heavy chain CDR3:

(SEQ ID NO: 74)

DGTMGIAYYFDY
Light chain CDR1:

(SEQ ID NO: 75)

KASQNINRYLN
Light chain CDR2:

(SEQ ID NO: 76)

NANSLQT
Light chain CDR3:

(SEQ ID NO: 77)

LQHNSWPYT

In an embodiment of the present invention, an anti-TMEM-180 antibody comprises all of the above CDRs. In an embodiment of the present invention, an anti-TMEM-180 antibody is a humanized antibody comprising all of the above CDRs.

One aspect of the anti-TMEM-180 antibody according to the present invention has at least one of the six CDRs indicated below. These CDRs are the CDR sequences of anti-TMEM-180 antibody clone 699 indicated in the examples to be subsequently described.

Heavy chain CDR1:

(SEQ ID NO: 80)

SYDIS
Heavy chain CDR2:

(SEQ ID NO: 81)

AINPGSGGTGYNEKFKGK
Heavy chain CDR3:

(SEQ ID NO: 82)

IHGGYRYWFAY
Light chain CDR1:

(SEQ ID NO: 83)

RASSSVSYMH
Light chain CDR2:

(SEQ ID NO: 84)

DTSKLAS
Light chain CDR3:

(SEQ ID NO: 85)

LQRSSYPPT

One aspect of the anti-TMEM-180 antibody according to the present invention has at least one of the six CDRs indicated below. These CDRs are the CDR sequences of anti-TMEM-180 antibody clone 1052 indicated in the examples to be subsequently described.

Heavy chain CDR1:

(SEQ ID NO: 88)

SNGVG
Heavy chain CDR2:

(SEQ ID NO: 89)

TIWTGGGTNYNSGVQS
Heavy chain CDR3:

(SEQ ID NO: 90)

EYMGFDY
Light chain CDR1:

(SEQ ID NO: 91)

KASQNVGINVG
Light chain CDR2:

(SEQ ID NO: 92)

WASNRDT
Light chain CDR3:

(SEQ ID NO: 93)

LQHNSYPRT

One aspect of the anti-TMEM-180 antibody according to the present invention has at least one of the six CDRs indicated below. These CDRs are the CDR sequences of anti-TMEM-180 antibody clone 1105 indicated in the examples to be subsequently described.

Heavy chain CDR1:

(SEQ ID NO: 96)

SNGVG
Heavy chain CDR2:

(SEQ ID NO: 97)

TIWSGGGTNYNSAVQS
Heavy chain CDR3:

(SEQ ID NO: 98)

EEKGFAY
Light chain CDR1:

(SEQ ID NO: 99)

KASQNVGINVG
Light chain CDR2:

(SEQ ID NO: 100)

WASNRDT
Light chain CDR3:

(SEQ ID NO: 101)

LQHNSYPRA

In an embodiment of the present invention, an anti-TMEM-180 antibody comprises at least one of the six CDRs shown below. These CDRs are CDR sequences of the 1361-5 clone antibody shown in the examples described below.

a heavy chain CDR1:

(SEQ ID NO: 171)

NYWMT
a heavy chain CDR2:

(SEQ ID NO: 172)

SITNTGGSTAYPDSV
a heavy chain CDR3:

(SEQ ID NO: 173)

AGYSSYPDYFDY
a light chain CDR1:

(SEQ ID NO: 174)

KAGQNIYNYLA
a light chain CDR2:

(SEQ ID NO: 175)

NANSLQT
a light chain CDR3:

(SEQ ID NO: 176)

QQYSSGWT

In an embodiment of the present invention, an anti-TMEM-180 antibody is an antibody which compete with the antibody in this embodiment for binding to TMEM-180.

In an embodiment of the present invention, an anti-TMEM-180 antibody comprises all of the six CDRs below. These CDRs are CDR sequences of the 1361-5 clone antibody shown in the examples described below.

a heavy chain CDR1:

(SEQ ID NO: 171)

NYWMT
a heavy chain CDR2:

(SEQ ID NO: 172)

SITNTGGSTAYPDSV
a heavy chain CDR3:

(SEQ ID NO: 173)

AGYSSYPDYFDY
a light chain CDR1:

(SEQ ID NO: 174)

KAGQNIYNYLA
a light chain CDR2:

(SEQ ID NO: 175)

NANSLQT
a light chain CDR3:

(SEQ ID NO: 176)

QQYSSGWT

In an embodiment of the present invention, an anti-TMEM-180 antibody is an antibody which compete with the antibody in this embodiment for binding to TMEM-180.

In an embodiment of the present invention, an anti-TMEM-180 antibody comprises a heavy chain variable region (SEQ ID NO: 177) and a light chain variable region (SEQ ID NO: 178).

In an embodiment of the present invention, an anti-TMEM-180 antibody is an antibody which compete with the antibody in this embodiment for binding to TMEM-180.

In an embodiment of the present invention, an anti-TMEM-180 antibody has a heavy chain (SEQ ID NO: 180) and a light chain (SEQ ID NO: 182). In an embodiment of the present invention, an anti-TMEM-180 antibody is an antibody which compete with the antibody in this embodiment for binding to TMEM-180.

In each of the above-mentioned aspects, although the anti-TMEM-180 antibody may contain any of the six CDRs provided the effects of the present invention are demonstrated, it can also contain, for example, two or more, three or more, four or more, five or more or all six of the six CDRs.

In each of the above-mentioned aspects, at least one of heavy chain CDR1 to CDR3 and light chain CDR1 to CDR3 may contain one to several amino acid additions, substitutions or deletions.

In the present description, an “amino acid” is used in the broadest sense, and includes artificial amino acid variants and derivatives in addition to naturally-occurring amino acids. Amino acids may be indicated using customary one-letter or three-letter codes. In the present description, examples of amino acids or derivatives thereof include natural protein L-amino acids, unnatural amino acids, and chemically synthesized compounds having properties widely known in the art to be properties of amino acids. Examples of unnatural amino acids include, but are not limited to, α,α-disubstituted amino acids (such as α-methylalanine), N-alkyl-α-amino acids, D-amino acids, β-amino acids and α-hydroxy acids having main chain structures that differ from those occurring naturally, amino acids having side chain structures that differ from those occurring naturally (such as norleucine or homohistidine), amino acids having a surplus methylene in a side chain thereof (such as homoamino acid, homophenylalanine or homohistidine), and amino acids in which a carboxylic acid functional group has been substituted with a sulfonic acid group in a side chain thereof (such as cysteic acid).

In the present description, in the case of referring to “having one to several amino acid additions, substitutions or deletions”, although there are no particular limitations on the number of, for example, added, substituted or deleted amino acids provided the polypeptide obtained as a result thereof retains the function of a CDR, examples thereof include one, two, three or four amino acid additions, substitutions or deletions. The substituted or added amino acids may be unnatural amino acids or amino acid analogs in addition to natural protein amino acids. The location of an amino acid deletion, substitution or addition may be at any location of the original CDR sequence provided the function of a CDR is retained.

In each of the aspects of the above-mentioned anti-TMEM-180 antibody, at least one of the heavy chain CDR1 to CDR3 and light chain CDR1 to CDR3 may have an amino acid sequence having identity of 80% or more with the original amino acid sequences of the heavy chain CDR1 to CDR3 and light chain CDR1 to CDR3.

In the present description, “having identity of 80% or more” means that the number of common amino acid residues is 80% or more of the amino acids of the original sequence when the amino acid sequence of a polypeptide having the original sequence is aligned with the amino acid sequence of a polypeptide having a mutated sequence so that there is maximum agreement between the two.

Identity may be of any percentage of 80% or more that retains the function of a CDR, and can be, for example, 85% or more, 90% or more, 95% or more, 98% or more or 99% or more.

A CDR comprising an amino acid sequence in which an amino acid has been added, substituted or deleted in the amino acid sequences of heavy chain CDR1 to CDR3 and light chain CDR1 to CDR3, or a CDR having sequence identity of 80% or more with the amino acid sequences of heavy chain CDR1 to CDR3 and light chain CDR1 to CDR3, can be produced using a known method such as site-specific mutagenesis, random mutagenesis, chain shuffling or CDR walking. According to these methods, antibody or antigen fragments having various mutations in the CDR thereof can be presented on a phage surface by phage display, and CDRs having a higher degree of affinity maturation are well known among persons with ordinary skill in the art to be obtained by screening using antigen (see, for example, Wu, et al., PNAS, 95: 6037-6042 (1998); Schier, R., et al., J. Mol. Biol., 263, 551-567 (1996); Schier, R., et al., J. Mol. Biol., 255, 28-43 (1996); Yang, W. P., et al., J. Mol. Biol., 254, 392-403 (1995)).

Another aspect of the anti-TMEM-180 antibody according to the present invention contains:
a heavy chain containing the amino acid sequence represented by SEQ ID NO: 13;
a heavy chain containing an amino acid sequence containing one to several amino acid additions, substitutions or deletions in the amino acid sequence represented by SEQ ID NO: 13; or
a heavy chain containing an amino acid sequence having identity of 80% or more with the amino acid sequence represented by SEQ ID NO: 13.

The amino acid sequence represented by SEQ ID NO: 13 is an amino acid sequence of the heavy chain variable region of anti-TMEM-180 antibody clone 98.

In the present description, in the case of one to several amino acid additions, substitutions or deletions in the amino acid sequence of the heavy chain or light chain, the number of amino acids that are added, substituted or deleted can be, for example, one, two, three, four, five, six, seven, eight, nine, or ten. Other terms are as previously described.

Another aspect of the anti-TMEM-180 antibody according to the present invention contains:
a light chain containing the amino acid sequence represented by SEQ ID NO: 14;
a light chain containing an amino acid sequence containing one to several amino acid additions, substitutions or deletions in the amino acid sequence represented by SEQ ID NO: 14; or
a light chain containing an amino acid sequence having identity of 80% or more with the amino acid sequence represented by SEQ ID NO: 14.

The amino acid sequence represented by SEQ ID NO: 14 is an amino acid sequence of the light chain variable region of anti-TMEM-180 antibody clone 98.

The anti-TMEM-180 antibody according to the present invention may contain a heavy chain containing the amino acid sequence represented by SEQ ID NO: 13, a heavy chain containing an amino acid sequence containing one to several amino acid additions, substitutions or deletions in the amino acid sequence represented by SEQ ID NO: 13, or a heavy chain containing an amino acid sequence having identity of 80% or more with the amino acid sequence represented by SEQ ID NO: 13, and
a light chain containing the amino acid sequence represented by SEQ ID NO: 14, a light chain containing an amino acid sequence containing one to several amino acid additions, substitutions or deletions in the amino acid sequence represented by SEQ ID NO: 14, or a light chain containing an amino acid sequence having identity of 80% or more with the amino acid sequence represented by SEQ ID NO: 14.

Another aspect of the anti-TMEM-180 antibody according to the present invention contains:
a heavy chain containing the amino acid sequence represented by SEQ ID NO: 15;
a heavy chain containing an amino acid sequence containing one to several amino acid additions, substitutions or deletions in the amino acid sequence represented by SEQ ID NO: 15; or
a heavy chain containing an amino acid sequence having identity of 80% or more with the amino acid sequence represented by SEQ ID NO: 15.

The amino acid sequence represented by SEQ ID NO: 15 is an amino acid sequence of the heavy chain variable region of anti-TMEM-180 antibody clone 101.

Another aspect of the anti-TMEM-180 antibody according to the present invention contains:
a light chain containing the amino acid sequence represented by SEQ ID NO: 16;
a light chain containing an amino acid sequence containing one to several amino acid additions, substitutions or deletions in the amino acid sequence represented by SEQ ID NO: 16; or
a light chain containing an amino acid sequence having identity of 80% or more with the amino acid sequence represented by SEQ ID NO: 16.

The amino acid sequence represented by SEQ ID NO: 16 is an amino acid sequence of the light chain variable region of anti-TMEM-180 antibody clone 101.

Another aspect of the anti-TMEM-180 antibody according to the present invention may contain a heavy chain containing the amino acid sequence represented by SEQ ID NO: 15, a heavy chain containing an amino acid sequence containing one to several amino acid additions, substitutions or deletions in the amino acid sequence represented by SEQ ID NO: 15, or a heavy chain containing an amino acid sequence having identity of 80% or more with the amino acid sequence represented by SEQ ID NO: 15, and
a light chain containing the amino acid sequence represented by SEQ ID NO: 16, a light chain containing an amino acid sequence containing one to several amino acid additions, substitutions or deletions in the amino acid sequence represented by SEQ ID NO: 16, or a light chain containing an amino acid sequence having identity of 80% or more with the amino acid sequence represented by SEQ ID NO: 16.

Another aspect of the anti-TMEM-180 antibody according to the present invention contains:
a heavy chain containing the amino acid sequence represented by SEQ ID NO: 46;
a heavy chain containing an amino acid sequence containing one to several amino acid additions, substitutions or deletions in the amino acid sequence represented by SEQ ID NO: 46; or
a heavy chain containing an amino acid sequence having identity of 80% or more with the amino acid sequence represented by SEQ ID NO: 46.

The amino acid sequence represented by SEQ ID NO: 46 is an amino acid sequence of the heavy chain variable region of anti-TMEM-180 antibody clone 212.

Another aspect of the anti-TMEM-180 antibody according to the present invention contains:
a light chain containing the amino acid sequence represented by SEQ ID NO: 47;
a light chain containing an amino acid sequence containing one to several amino acid additions, substitutions or deletions in the amino acid sequence represented by SEQ ID NO: 47; or
a light chain containing an amino acid sequence having identity of 80% or more with the amino acid sequence represented by SEQ ID NO: 47.

The amino acid sequence represented by SEQ ID NO: 47 is an amino acid sequence of the light chain variable region of anti-TMEM-180 antibody clone 212.

Another aspect of the anti-TMEM-180 antibody according to the present invention may contain a heavy chain containing the amino acid sequence represented by SEQ ID NO: 46, a heavy chain containing an amino acid sequence containing one to several amino acid additions, substitutions or deletions in the amino acid sequence represented by SEQ ID NO: 46, or a heavy chain containing an amino acid sequence having identity of 80% or more with the amino acid sequence represented by SEQ ID NO: 46, and
a light chain containing the amino acid sequence represented by SEQ ID NO: 47, a light chain containing an amino acid sequence containing one to several amino acid additions, substitutions or deletions in the amino acid sequence represented by SEQ ID NO: 47, or a light chain containing an amino acid sequence having identity of 80% or more with the amino acid sequence represented by SEQ ID NO: 47.

Another aspect of the anti-TMEM-180 antibody according to the present invention contains:
a heavy chain containing the amino acid sequence represented by SEQ ID NO: 54;
a heavy chain containing an amino acid sequence containing one to several amino acid additions, substitutions or deletions in the amino acid sequence represented by SEQ ID NO: 54; or
a heavy chain containing an amino acid sequence having identity of 80% or more with the amino acid sequence represented by SEQ ID NO: 54.

The amino acid sequence represented by SEQ ID NO: 54 is an amino acid sequence of the heavy chain variable region of anti-TMEM-180 antibody clone 129.

Another aspect of the anti-TMEM-180 antibody according to the present invention contains:
a light chain containing the amino acid sequence represented by SEQ ID NO: 55;
a light chain containing an amino acid sequence containing one to several amino acid additions, substitutions or deletions in the amino acid sequence represented by SEQ ID NO: 55; or
a light chain containing an amino acid sequence having identity of 80% or more with the amino acid sequence represented by SEQ ID NO: 55.

The amino acid sequence represented by SEQ ID NO: 55 is an amino acid sequence of the light chain variable region of anti-TMEM-180 antibody clone 129.

Another aspect of the anti-TMEM-180 antibody according to the present invention may contain a heavy chain containing the amino acid sequence represented by SEQ ID NO: 54, a heavy chain containing an amino acid sequence containing one to several amino acid additions, substitutions or deletions in the amino acid sequence represented by SEQ ID NO: 54, or a heavy chain containing an amino acid sequence having identity of 80% or more with the amino acid sequence represented by SEQ ID NO: 54, and
a light chain containing the amino acid sequence represented by SEQ ID NO: 55, a light chain containing an amino acid sequence containing one to several amino acid additions, substitutions or deletions in the amino acid sequence represented by SEQ ID NO: 55, or a light chain containing an amino acid sequence having identity of 80% or more with the amino acid sequence represented by SEQ ID NO: 55.

Another aspect of the anti-TMEM-180 antibody according to the present invention contains:
a heavy chain containing the amino acid sequence represented by SEQ ID NO: 62;
a heavy chain containing an amino acid sequence containing one to several amino acid additions, substitutions or deletions in the amino acid sequence represented by SEQ ID NO: 62; or
a heavy chain containing an amino acid sequence having identity of 80% or more with the amino acid sequence represented by SEQ ID NO: 62.

The amino acid sequence represented by SEQ ID NO: 62 is an amino acid sequence of the heavy chain variable region of anti-TMEM-180 antibody clone 382.

Another aspect of the anti-TMEM-180 antibody according to the present invention contains:
a light chain containing the amino acid sequence represented by SEQ ID NO: 63;
a light chain containing an amino acid sequence containing one to several amino acid additions, substitutions or deletions in the amino acid sequence represented by SEQ ID NO: 63; or
a light chain containing an amino acid sequence having identity of 80% or more with the amino acid sequence represented by SEQ ID NO: 63.

The amino acid sequence represented by SEQ ID NO: 63 is an amino acid sequence of the light chain variable region of anti-TMEM-180 antibody clone 382.

Another aspect of the anti-TMEM-180 antibody according to the present invention may contain a heavy chain containing the amino acid sequence represented by SEQ ID NO: 62, a heavy chain containing an amino acid sequence containing one to several amino acid additions, substitutions or deletions in the amino acid sequence represented by SEQ ID NO: 62, or a heavy chain containing an amino acid sequence having identity of 80% or more with the amino acid sequence represented by SEQ ID NO: 62, and
a light chain containing the amino acid sequence represented by SEQ ID NO: 63, a light chain containing an amino acid sequence containing one to several amino acid additions, substitutions or deletions in the amino acid sequence represented by SEQ ID NO: 63, or a light chain containing an amino acid sequence having identity of 80% or more with the amino acid sequence represented by SEQ ID NO: 63.

Another aspect of the anti-TMEM-180 antibody according to the present invention contains:
a heavy chain containing the amino acid sequence represented by SEQ ID NO: 70;
a heavy chain containing an amino acid sequence containing one to several amino acid additions, substitutions or deletions in the amino acid sequence represented by SEQ ID NO: 70; or
a heavy chain containing an amino acid sequence having identity of 80% or more with the amino acid sequence represented by SEQ ID NO: 70.

The amino acid sequence represented by SEQ ID NO: 70 is an amino acid sequence of the heavy chain variable region of anti-TMEM-180 antibody clone 1361.

Another aspect of the anti-TMEM-180 antibody according to the present invention contains:
a light chain containing the amino acid sequence represented by SEQ ID NO: 71;
a light chain containing an amino acid sequence containing one to several amino acid additions, substitutions or deletions in the amino acid sequence represented by SEQ ID NO: 71; or
a light chain containing an amino acid sequence having identity of 80% or more with the amino acid sequence represented by SEQ ID NO: 71.

The amino acid sequence represented by SEQ ID NO: 71 is an amino acid sequence of the light chain variable region of anti-TMEM-180 antibody clone 1361.

Another aspect of the anti-TMEM-180 antibody according to the present invention may contain a heavy chain containing the amino acid sequence represented by SEQ ID NO: 70, a heavy chain containing an amino acid sequence containing one to several amino acid additions, substitutions or deletions in the amino acid sequence represented by SEQ ID NO: 70, or a heavy chain containing an amino acid sequence having identity of 80% or more with the amino acid sequence represented by SEQ ID NO: 70, and
a light chain containing the amino acid sequence represented by SEQ ID NO: 71, a light chain containing an amino acid sequence containing one to several amino acid additions, substitutions or deletions in the amino acid sequence represented by SEQ ID NO: 71, or a light chain containing an amino acid sequence having identity of 80% or more with the amino acid sequence represented by SEQ ID NO: 71.

Another aspect of the anti-TMEM-180 antibody according to the present invention contains:
a heavy chain containing the amino acid sequence represented by SEQ ID NO: 78;
a heavy chain containing an amino acid sequence containing one to several amino acid additions, substitutions or deletions in the amino acid sequence represented by SEQ ID NO: 78; or
a heavy chain containing an amino acid sequence having identity of 80% or more with the amino acid sequence represented by SEQ ID NO: 78.

The amino acid sequence represented by SEQ ID NO: 78 is an amino acid sequence of the heavy chain variable region of anti-TMEM-180 antibody clone 669.

Another aspect of the anti-TMEM-180 antibody according to the present invention contains:
a light chain containing the amino acid sequence represented by SEQ ID NO: 79;
a light chain containing an amino acid sequence containing one to several amino acid additions, substitutions or deletions in the amino acid sequence represented by SEQ ID NO: 79; or
a light chain containing an amino acid sequence having identity of 80% or more with the amino acid sequence represented by SEQ ID NO: 79.

The amino acid sequence represented by SEQ ID NO: 79 is an amino acid sequence of the light chain variable region of anti-TMEM-180 antibody clone 669.

Another aspect of the anti-TMEM-180 antibody according to the present invention may contain a heavy chain containing the amino acid sequence represented by SEQ ID NO: 78, a heavy chain containing an amino acid sequence containing one to several amino acid additions, substitutions or deletions in the amino acid sequence represented by SEQ ID NO: 78, or a heavy chain containing an amino acid sequence having identity of 80% or more with the amino acid sequence represented by SEQ ID NO: 78, and
a light chain containing the amino acid sequence represented by SEQ ID NO: 79, a light chain containing an amino acid sequence containing one to several amino acid additions, substitutions or deletions in the amino acid sequence represented by SEQ ID NO: 79, or a light chain containing an amino acid sequence having identity of 80% or more with the amino acid sequence represented by SEQ ID NO: 79.

Another aspect of the anti-TMEM-180 antibody according to the present invention contains:
a heavy chain containing the amino acid sequence represented by SEQ ID NO: 86;
a heavy chain containing an amino acid sequence containing one to several amino acid additions, substitutions or deletions in the amino acid sequence represented by SEQ ID NO: 86; or
a heavy chain containing an amino acid sequence having identity of 80% or more with the amino acid sequence represented by SEQ ID NO: 86.

The amino acid sequence represented by SEQ ID NO: 86 is an amino acid sequence of the heavy chain variable region of anti-TMEM-180 antibody clone 699.

Another aspect of the anti-TMEM-180 antibody according to the present invention contains:
a light chain containing the amino acid sequence represented by SEQ ID NO: 87;
a light chain containing an amino acid sequence containing one to several amino acid additions, substitutions or deletions in the amino acid sequence represented by SEQ ID NO: 87; or
a light chain containing an amino acid sequence having identity of 80% or more with the amino acid sequence represented by SEQ ID NO: 87.

The amino acid sequence represented by SEQ ID NO: 87 is an amino acid sequence of the light chain variable region of anti-TMEM-180 antibody clone 699.

Another aspect of the anti-TMEM-180 antibody according to the present invention may contain a heavy chain containing the amino acid sequence represented by SEQ ID NO: 86, a heavy chain containing an amino acid sequence containing one to several amino acid additions, substitutions or deletions in the amino acid sequence represented by SEQ ID NO: 86, or a heavy chain containing an amino acid sequence having identity of 80% or more with the amino acid sequence represented by SEQ ID NO: 86, and
a light chain containing the amino acid sequence represented by SEQ ID NO: 87, a light chain containing an amino acid sequence containing one to several amino acid additions, substitutions or deletions in the amino acid sequence represented by SEQ ID NO: 87, or a light chain containing an amino acid sequence having identity of 80% or more with the amino acid sequence represented by SEQ ID NO: 87.

Another aspect of the anti-TMEM-180 antibody according to the present invention contains:
a heavy chain containing the amino acid sequence represented by SEQ ID NO: 94;
a heavy chain containing an amino acid sequence containing one to several amino acid additions, substitutions or deletions in the amino acid sequence represented by SEQ ID NO: 94; or
a heavy chain containing an amino acid sequence having identity of 80% or more with the amino acid sequence represented by SEQ ID NO: 94.

The amino acid sequence represented by SEQ ID NO: 94 is an amino acid sequence of the heavy chain variable region of anti-TMEM-180 antibody clone 1052.

Another aspect of the anti-TMEM-180 antibody according to the present invention contains:
a light chain containing the amino acid sequence represented by SEQ ID NO: 95;
a light chain containing an amino acid sequence containing one to several amino acid additions, substitutions or deletions in the amino acid sequence represented by SEQ ID NO: 95; or
a light chain containing an amino acid sequence having identity of 80% or more with the amino acid sequence represented by SEQ ID NO: 95.

The amino acid sequence represented by SEQ ID NO: 95 is an amino acid sequence of the light chain variable region of anti-TMEM-180 antibody clone 1052.

Another aspect of the anti-TMEM-180 antibody according to the present invention may contain a heavy chain containing the amino acid sequence represented by SEQ ID NO: 94, a heavy chain containing an amino acid sequence containing one to several amino acid additions, substitutions or deletions in the amino acid sequence represented by SEQ ID NO: 94, or a heavy chain containing an amino acid sequence having identity of 80% or more with the amino acid sequence represented by SEQ ID NO: 94, and
a light chain containing the amino acid sequence represented by SEQ ID NO: 95, a light chain containing an amino acid sequence containing one to several amino acid additions, substitutions or deletions in the amino acid sequence represented by SEQ ID NO: 95, or a light chain containing an amino acid sequence having identity of 80% or more with the amino acid sequence represented by SEQ ID NO: 95.

Another aspect of the anti-TMEM-180 antibody according to the present invention contains:
a heavy chain containing the amino acid sequence represented by SEQ ID NO: 102;
a heavy chain containing an amino acid sequence containing one to several amino acid additions, substitutions or deletions in the amino acid sequence represented by SEQ ID NO: 102; or
a heavy chain containing an amino acid sequence having identity of 80% or more with the amino acid sequence represented by SEQ ID NO: 102.

The amino acid sequence represented by SEQ ID NO: 102 is an amino acid sequence of the heavy chain variable region of anti-TMEM-180 antibody clone 1105.

Another aspect of the anti-TMEM-180 antibody according to the present invention contains:
a light chain containing the amino acid sequence represented by SEQ ID NO: 103;
a light chain containing an amino acid sequence containing one to several amino acid additions, substitutions or deletions in the amino acid sequence represented by SEQ ID NO: 103; or
a light chain containing an amino acid sequence having identity of 80% or more with the amino acid sequence represented by SEQ ID NO: 103.

The amino acid sequence represented by SEQ ID NO: 103 is an amino acid sequence of the light chain variable region of anti-TMEM-180 antibody clone 1105.

Another aspect of the anti-TMEM-180 antibody according to the present invention may contain a heavy chain containing the amino acid sequence represented by SEQ ID NO: 102, a heavy chain containing an amino acid sequence containing one to several amino acid additions, substitutions or deletions in the amino acid sequence represented by SEQ ID NO: 102, or a heavy chain containing an amino acid sequence having identity of 80% or more with the amino acid sequence represented by SEQ ID NO: 102, and
a light chain containing the amino acid sequence represented by SEQ ID NO: 103, a light chain containing an amino acid sequence containing one to several amino acid additions, substitutions or deletions in the amino acid sequence represented by SEQ ID NO: 103, or a light chain containing an amino acid sequence having identity of 80% or more with the amino acid sequence represented by SEQ ID NO: 103.

In an embodiment of the present invention, an anti-TMEM-180 antibody comprises
a heavy chain containing the amino acid sequence represented by SEQ ID NO: 180;
a heavy chain containing an amino acid sequence having one to several amino acid additions, substitutions or deletions in the amino acid sequence represented by SEQ ID NO: 180; or
a heavy chain containing an amino acid sequence having identity of 80% or more with the amino acid sequence represented by SEQ ID NO: 180.

The amino acid sequence represented by SEQ ID NO: 180 is the amino acid sequence of the heavy chain variable region of the 1361-5 clone anti-TMEM-180 antibody.

In an embodiment of the present invention, an anti-TMEM-180 antibody comprises
a light chain containing the amino acid sequence represented by SEQ ID NO: 182;
a light chain containing an amino acid sequence having one to several amino acid additions, substitutions or deletions in the amino acid sequence represented by SEQ ID NO: 182; or
a light chain containing an amino acid sequence having identity of 80% or more with the amino acid sequence represented by SEQ ID NO: 182.

The amino acid sequence represented by SEQ ID NO: 182 is the amino acid sequence of the light chain variable region of the 1361-5 clone anti-TMEM-180

In an embodiment of the present invention, an anti-TMEM-180 antibody may comprise
a heavy chain containing the amino acid sequence represented by SEQ ID NO: 180;
a heavy chain containing an amino acid sequence having one to several amino acid additions, substitutions or deletions in the amino acid sequence represented by SEQ ID NO: 180; or
a heavy chain containing an amino acid sequence having identity of 80% or more with the amino acid sequence represented by SEQ ID NO: 180; and
a light chain containing the amino acid sequence represented by SEQ ID NO: 182;
a light chain containing an amino acid sequence having one to several amino acid additions, substitutions or deletions in the amino acid sequence represented by SEQ ID NO: 182; or
a light chain containing an amino acid sequence having identity of 80% or more with the amino acid sequence represented by SEQ ID NO: 182.

In another embodiment of the present invention, an anti-TMEM-180 antibody may be an antibody that compete with the anti-TMEM-180 antibody of the present invention for binding to TMEM-180. In this specific embodiment, the anti-TMEM-180 antibody may not be an antibody comprising the amino acid sequences represented by SEQ ID NOs: 64 to 66 for heavy chain CDRs and the amino acid sequence represented by SEQ ID NOs: 67 to 69 for light chain CDRs.

In the antibody of the present invention or an antibody that compete with the antibody of the present invention for binding to TMEM-180, Fc region may have one or more amino acid substitutions. For example, the heavy chain may have, but not limited to, an amino acid substitution of serine to aspartic acid in the amino acid corresponding to the position 262 in SEQ ID NO: 180, an amino acid substitution of alanine to leucine in the amino acid corresponding to the position 353 of SEQ ID NO: 180, and an amino acid substitution of leucine to glutamine in the amino acid corresponding to the position 355 of SEQ ID NO: 180. For example, the heavy chain may further have, but not limited to, an amino acid substitution of serine to aspartic acid in the amino acid corresponding to the position 262 in SEQ ID NO: 180, an amino acid substitution of leucine to glutamine in the amino acid corresponding to the position 355 in SEQ ID NO: 180, and an amino acid substitution of glutamate to alanine in the amino acid corresponding to the position 356 in SEQ ID NO: 180.

The anti-TMEM-180 antibody according to the present invention may be an antibody in which one or more N-linked oligosaccharides are bound to the Fc region, and fucose is not bound to N-acetylglucosamine on the reducing end of the N-linked oligosaccharide.

For example, two N-linked oligosaccharide binding sites are present in the Fc region of IgG antibody and a complex oligosaccharide is bound to this site. N-linked oligosaccharides refer to oligosaccharides bound to Asn of the sequence Asn-X-Ser/Thr that have the common structure of Man3GlcNAc2-Asn. These are classified into high mannose types, mixed types, complex types and the like depending on the type of oligosaccharide that binds to the two mannose (Man) on the non-reducing end.

Although fucose can be bound to N-acetylglucosamine (GlcNAc) on the reducing end of N-linked oligosaccharides, ADCC activity is known to increase considerably in the case fucose is not bound to this site in comparison with the case in which it is bound thereto. This finding is described in, for example, WO 2002/031140, and the disclosure thereof is incorporated in the present description in its entirety by reference.

Since the dosage of an antibody can be reduced in the case of using as a pharmaceutical due to this considerable increase in ADCC activity, in addition to making it possible to reduce adverse side effects, medical expenses can also be reduced.

One aspect of the anticancer drug according to the present invention contains, as an active ingredient thereof, an anti-TMEM-180 antibody having a substance having anticancer activity bound thereto, or an antigen-binding fragment thereof. These substances are also referred to as antibody-drug conjugates (ADC). In this aspect, the anti-TMEM-180 antibody or antigen-binding fragment thereof may also have anticancer activity per se or may only have the ability to bind to TMEM-180 without exhibiting anticancer activity.

In the present description, a “substance having anticancer activity” refers to a substance that causes at least one of a reduction (delay or interruption) of tumor size, inhibition of tumor metastasis, inhibition (delay or interruption) of tumor growth and alleviation of one or multiple symptoms associated with cancer. More specifically, examples thereof include, but are not limited to, toxins, anticancer drugs and radioisotopes.

Examples of toxins having anticancer activity include Pseudomonas exotoxin (PE) and cytotoxic fragments thereof (such as PE38), diphtheria toxin and lysin A. Since these toxins having anticancer activity demonstrate toxicity only in cells recognized by the anti-TMEM-180 antibody, namely cancer cells expressing TMEM-180, they offer the advantage of allowing the obtaining of specific effects without having a detrimental effect on surrounding cells.

Examples of anticancer drugs include low molecular weight compounds such as adriamycin, daunomycin, mitomycin, cisplatin, vincristine, epirubicin, methotrexate, 5-fluorouracil, aclacinomycin, nitrogen mustard, cyclophosphamide, bleomycin, daunorubicin, doxorubicin, vincristine, vinblastine, vindesine, tamoxifen, monomethylauristatin E, SN-38, or dexamethasone, and proteins such as cytokines that activate immunocompetent cells (such as human interleukin 2, human granulocyte-macrophage colony stimulating factor, human macrophage colony stimulating factor or human interleukin 12).

Examples of radioisotopes having anticancer activity include ³²P, ¹⁴C, ¹²⁵I, ³H, ¹³¹I, ²¹¹At and ⁹⁰Y.

Substances having anticancer activity are able to bind to the anti-TMEM-180 antibody either directly or through a linker (for example, a cleavable linker) according to a known method or method in compliance therewith. A substance having anticancer activity may be enclosed in a carrier such as a micelle or liposome and then bind the liposome to the anti-TMEM-180 antibody or an antigen-binding fragment thereof.

In the case the above-mentioned substance having anticancer activity is a protein or polypeptide, it may also be expressed in the form of a fusion protein formed of the substance having anticancer activity and an anti-TMEM-180 antibody by linking a nucleic acid encoding the anti-TMEM-180 antibody of the present invention to be subsequently described, encoding a substance having anticancer activity using DNA, and then inserting into a suitable expression vector.

In the present description, an “anticancer drug” refers to a pharmaceutical that causes at least one of a reduction (delay or interruption) of tumor size, inhibition of tumor metastasis, inhibition (delay or interruption) of tumor growth and alleviation of one or multiple symptoms associated with cancer. The term “anticancer drug” as used herein is interchangeably used with the term “pharmaceutical composition for use in treating cancer”.

The anticancer drug according to the present invention may also contain a pharmaceutically acceptable carrier or additive in addition to the active ingredient.

Examples of carriers and additives include, but are not limited to, pharmaceutically acceptable organic solvents such as water, saltwater, phosphate buffer, dextrose, glycerol and ethanol, as well as collagen, polyvinyl alcohol, polyvinylpyrrolidone, carboxyvinyl polymer, carboxymethyl cellulose sodium, sodium polyacrylate, sodium alginate, water-soluble dextran, sodium carboxymethyl starch, pectin, methyl cellulose, ethyl cellulose, xanthan gum, gum arabic, casein, agar, polyethylene glycol, diglycerin, glycerin, propylene glycol, Vaseline, paraffin, stearyl alcohol, stearic acid, human serum albumin, mannitol, sorbitol, lactose and surfactants.

The pharmaceutical composition of the present invention can adopt various forms, such as a liquid (e.g. injection preparation), dispersion, suspension, tablet, pill, powder or suppository. The pharmaceutical composition is preferably in the form of an injection preparation, and is preferably administered parenterally (such as intravenously, percutaneously, intraperitoneally or intramuscularly).

The pharmaceutical composition of the present invention is effective for treatment of cancer expressing TMEM-180. Examples of cancer expressing TMEM-180 include, but are not limited to, colon cancer and brain tumors. Examples of cancer include, but not limited to, solid cancers, for example, colon cancer, brain tumor, gastric cancer, pancreas cancer, breast cancer, prostate cancer, kidney cancer, and bladder cancer.

The pharmaceutical composition of the present invention is a pharmaceutical composition for use in treating cancer in a subject having the cancer, comprising an antibody that binds to TMEM-180. In an embodiment of the present invention, the subject to be treated may be a subject that has a cancer expressing TMEM-180. In an embodiment of the present invention, the subject to be treated may be a subject wherein TMEM-180 has been detected in the body fluid sample of the subject. In an embodiment of the present invention, the subject to be treated may be a subject wherein exosomes expressing TMEM-180 on their surface have been detected in the body fluid sample of the subject.

The present invention also includes a method for treating cancer comprising administering the anticancer drug according to the present invention. In an aspect of the present invention, provided is a method for treating cancer in a subject having the cancer, comprising administrating an antibody that binds to TMEM-180 to the subject.

The dosage of the anticancer drug of the present invention is such that, for example, the dosage of the anti-TMEM-180 antibody or antigen-binding fragment thereof is 0.025 mg/kg to 50 mg/kg, preferably 0.1 mg/kg to 0 mg/kg, more preferably 0.1 mg/kg to 25 mg/kg, and even more preferably 0.1 mg/kg to 10 mg/kg or 0.1 mg/kg to 3 mg/kg, although not limited thereto.

The anticancer drug according to the present invention may be combined with other cancer therapy. Examples of other cancer therapy include administration of the above-mentioned other anticancer drugs, radiotherapy, surgery and cancer vaccine therapy.

In the present description, “cancer vaccine therapy” refers to a method for preventing or treating cancer that involves enhancing the immunity of a patient per se against cancer cells by administering a cancer antigen to the patient or stimulating patient-derived immune cells in vitro with a cancer antigen and then returning the cells to the patient. A peptide derived from a protein specifically expressed by cancer cells (cancer antigen-derived peptide) is used as an example of a cancer antigen used in cancer vaccine therapy.

A cancer antigen-derived peptide activates cytotoxic T lymphocytes (CTL) as a result of binding to human leukocyte antigen (HLA) on the surface of antigen-presenting cells such as dendritic cells and being presented on the CTL. Activated CTL then attack and eliminate cancer cells expressing the same antigen as the peptide. HLA molecules have a high degree of genetic diversity and demonstrate different genotypes depending on the individual. The sequences of those peptides having the possibility of being derived from a certain cancer antigen protein and binding to an HLA molecule of a specific genotype can be determined with known software or the like.

For example, those peptides derived from TMEM-180 protein and having the possibility of binding to HLA type A2 or HLA type A24 commonly found among Japanese were predicted as indicated below using HLA Peptide Binding Predictions published by the Bioinformatics and Molecular Analysis Section of the U.S. National Institutes of Health (http://www-bimas.citnih.govicgi-bin/molbio/ken_parker_comboform). In the tables, “Start position” indicates the location in the amino acid sequence set forth in SEQ ID NO: 17.

According to cancer vaccine therapy using these peptides, since CTL can be activated to attack cancer cells expressing TMEM-180 protein, cancer cells can be efficiently attacked by using in combination with the anticancer drug according to the present invention that targets TMEM-180, thereby making it possible to prevent or treat cancer. Cancer vaccine compositions containing these peptides can be prepared by a person with ordinary skill in the art in accordance with known methods.

TABLE 1
HLA molecule type: A_0201
Cutoff Score: 10 (Estimate of Half Time of
Disassociation of a Molecule Containing
This Subsequence)

| Rank | Start Position | Sequence | SEQ ID NO |
|---|---|---|---|
| 1 | 132 | CLYDgFLTLV | 104 |
| 2 | 33 | FLLYyVDTFV | 105 |
| 3 | 266 | FLWFvSMDLV | 106 |
| 4 | 322 | FLSLcRRWGV | 107 |
| 5 | 339 | FLLKIGLSLL | 108 |
| 6 | 376 | KLLTIVVTDL | 109 |
| 7 | 401 | ALLFgMVALV | 110 |
| 8 | 23 | ALFTtILHNV | 111 |
| 9 | 9 | WLLGIPTAVV | 112 |
| 10 | 482 | QLFTwSQFTL | 113 |
| 11 | 109 | LALSfLAFWV | 114 |
| 12 | 342 | KLGLsLLMLL | 115 |
| 13 | 2 | GLGQpQAWLL | 116 |
| 14 | 490 | TLHGrRLHMV | 117 |
| 15 | 60 | LLWNsLNDPL | 118 |
| 16 | 64 | SLNDpLFGWL | 119 |
| 17 | 139 | TLVDIHHHAL | 120 |
| 18 | 49 | KMAFwVGETV | 121 |
| 19 | 469 | YLLVIVPITC | 122 |
| 20 | 505 | NLSQaQTLDV | 123 |
| 21 | 377 | LLTLvVTDLV | 124 |
| 22 | 294 | LLSDhISLST | 125 |
| 23 | 348 | LMLLaGPDHL | 126 |
| 24 | 258 | RQLArHRNFL | 127 |
| 25 | 130 | CLCLyDGFLT | 128 |
| 26 | 425 | LLCFyTGHDL | 129 |
| 27 | 472 | VLVPiTCALL | 130 |
| 28 | 350 | LLAGpDHLSL | 131 |
| 29 | 70 | FGWLsDRQFL | 132 |
| 30 | 324 | SLCRrWGVYA | 133 |
| 31 | 481 | LQLFtWSQFT | 134 |
| 32 | 78 | FLSSqPRSGA | 135 |
| 33 | 204 | GLGFIGATQL | 136 |
| 34 | 124 | GLQFILCLCL | 137 |
| 35 | 153 | ALSAhDRTHL | 138 |
| 36 | 212 | QLLRrRVEAA | 139 |
| 37 | 17 | VVYGsLALFT | 140 |
| 38 | 470 | LLVLvPITCA | 141 |
| 39 | 402 | LLFGmVALVT | 142 |
| 40 | 271 | SMDLvQVFHC | 143 |
| 41 | 147 | ALLAdLALSA | 144 |
| 42 | 332 | YAVVrGLFLL | 145 |
| 43 | 45 | YKINkMAFWV | 146 |
| 44 | 282 | FNSNfFPLFL | 147 |
| 45 | 471 | LVLVpITCAL | 148 |
| 46 | 88 | GLSSrAVVLA | 149 |
| 47 | 417 | FAPLIGTWLL | 150 |

TABLE 2
HLA molecule type: A24
Cutoff Score: 10 (Estimate of Half Time of
Disassociation of a Molecule Containing
This Subsequence)

| Rank | Start Position | Sequence | SEQ ID |
|---|---|---|---|
| 1 | 311 | SYVApHLNNL | 151 |
| 2 | 331 | VYAVvRGLFL | 152 |
| 3 | 180 | SYAFwNKEDF | 153 |
| 4 | 265 | NFLWfVSMDL | 154 |
| 5 | 286 | FFPLfLEHLL | 155 |
| 6 | 338 | LFLLkLGLSL | 156 |
| 7 | 416 | TFAPILGTWL | 157 |
| 8 | 369 | VFTEgTCKLL | 158 |
| 9 | 285 | NFFPIFLEHL | 159 |
| 10 | 51 | AFWVgETVFL | 160 |
| 11 | 281 | HFNSnFFPLF | 161 |
| 12 | 376 | KLLTIVVTDL | 162 |
| 13 | 32 | VFLLyYVDTF | 163 |
| 14 | 258 | RQLArHRNFL | 164 |
| 15 | 336 | RGLFILKLGL | 165 |
| 16 | 503 | RQNLsQAQTL | 166 |
| 17 | 24 | LFTTiLHNVF | 167 |
| 18 | 64 | SLNDpLFGWL | 168 |
| 19 | 277 | VFHChFNSNF | 169 |
| 20 | 69 | LFGWISDRQF | 170 |

(Nucleic Acid)

The present invention also includes a nucleic acid that encodes the anti-TMEM-180 antibody, or antigen-binding fragment thereof, according to the present invention. The nucleic acid may be a naturally-occurring nucleic acid or artificial nucleic acid, and examples thereof include, but are not limited to, DNA, RNA and chimeras of DNA and RNA. The base sequence of a nucleic acid encoding the anti-TMEM-180 antibody or antigen-binding fragment thereof can be determined by a person with ordinary skill in the art in accordance with a known method or method complying therewith, and can also be prepared using a known method or method complying therewith.

Examples of nucleic acids encoding the anti-TMEM-180 antibody or antigen-binding fragment thereof according to the present invention include, but are not limited to, nucleic acid containing DNA encoding a heavy chain or light chain of anti-TMEM-180 antibody clone 98, nucleic acid containing DNA encoding any of heavy chain CDR1 to CDR3 and light chain CDR1 to CDR3 of anti-TMEM-180 antibody clone 98, nucleic acid containing DNA encoding a heavy chain or light chain of anti-TMEM-180 antibody clone 101, nucleic acid containing DNA encoding any of heavy chain CDR1 to CDR3 and light chain CDR1 to CDR3 of anti-TMEM-180 antibody clone 101, nucleic acid containing DNA encoding a heavy chain or light chain of anti-TMEM-180 antibody clone 212, and nucleic acid containing DNA encoding any of heavy chain CDR1 to CDR3 and light chain CDR1 to CDR3 of anti-TMEM-180 antibody clone 212.

Examples of the nucleic acid encoding the anti-TMEM-180 antibody of the present invention or an antigen-binding fragment thereof include a nucleic acid comprising DNA encoding the heavy chain or the light chain of the 1361-5 clone antibody, a nucleic acid comprising any one of the heavy chain CDRs 1 to 3 and the light chain CDRs 1 to 3 (in particular, the heavy chain CDR2) of the 1361-5 clone antibody.

(Expression Vector)

The expression vector according to the present invention contains nucleic acid encoding the anti-TMEM-180 antibody, or antigen-binding fragment thereof, according to the present invention. The expression vector can be suitably selected according to the host cells used, and examples thereof include plant virus vectors such as a plasmid, retrovirus vector, adenovirus vector, adeno-associated virus (AAV) vector, cauliflower mosaic virus vector or tobacco mosaic virus vector, as well as a cosmid, YAC or EBV-derived episomal vector. Nucleic acid encoding the anti-TMEM-180 antibody of the present invention can be inserted into these expression vectors using a known method (such as a method using a restriction enzyme).

The expression vector according to the present invention can further contain a promoter for regulating expression of antibody gene, a replication origin or a selection marker gene and the like. The promoter and replication origin can be suitably selected according to the types of host cells and vector.

(Transformant)

The transformant according to the present invention contains the vector according to the present invention. A transformant can be obtained by transfecting suitable host cells with the vector of the present invention. Examples of host cells that can be used include eukaryotic cells in the manner of mammalian cells (such as CHO cells, COS cells, myeloma cells, HeLa cells or Vero cells), insect cells, plant cells and fungal cells (such as cells of fungi belonging to the genii Saccharomyces and Aspergillus), and prokaryotic cells in the manner of Escherichia coli (E. coli) or Bacillus subtilis cells.

(Antibody Production Method)

Although there are no particular limitations on the method used to produce the anti-TMEM-180 antibody or antigen-binding fragment thereof according to the present invention, anti-TMEM-180 monoclonal antibody, for example, can be obtained by isolating antibody-producing cells from a non-human mammal immunized with TMEM-180 or a fragment thereof, fusing these cells with myeloma cells or the like to produce a hybridoma, and then purifying antibody produced by this hybridoma. In addition, anti-TMEM-180 polyclonal antibody can be obtained from the serum of an animal immunized with TMEM-180 or a fragment thereof.

Immunization can be performed by immunizing an animal with TMEM-180 protein according to a common general technique. Immunization can be performed also by using exosomes expressing TMEM-180 on their surfaces instead of TMEM-180 protein. Antibodies obtained by immunization with exosomes expressing TMEM-180 on their surfaces may be suitable, but not limited, for detecting exosomes expressing TMEM-180 on their surfaces.

In the case of producing the anti-TMEM-180 antibody according to the present invention by genetic recombination, for example, a suitable host is transformed with an expression vector containing the nucleic acid according to the present invention and this transformant is then cultured under suitable conditions to express antibody followed by isolating and purifying the antibody in accordance with known methods.

Examples of isolation and purification methods include an affinity column using Protein A or the like, other chromatography column, filter, ultrafiltration, salting out and dialysis, and these methods can also be suitably combined.

Human chimeric antibody and human CDR-grafted antibody can be produced by cloning antibody gene from mRNA of a hybridoma that produces antibody of an animal other than a human, and then linking this with a portion of a human antibody gene using genetic recombination technology.

For example, in the case of human chimeric antibody, cDNA is synthesized from the mRNA of a hybridoma producing mouse antibody using reverse transcriptase, and the heavy chain variable region (VH) and light chain variable region (LH) are cloned by PCR followed by analysis of the sequences thereof. Next, 5′-primers containing a leader sequence are produced starting with the antibody base sequence having the highest identity rate, and the region from the signal sequence to the 3′-end of the variable region is cloned by PCR from the above-mentioned cDNA by the 5′-primer and variable region 3′-primer. On the other hand, the constant regions of the heavy chain and light chain of human IgG1 are cloned, and the variable portions derived from mouse antibody and the constant regions derived from human antibody are linked for each of heavy chain and light chain by PCR using the overlapping hanging method and then amplified. The resulting DNA can then be inserted into a suitable vector followed by transformation thereof to obtain human chimeric antibody.

In the case of CDR-grafted antibody, the variable portion of mouse antibody used and the variable portion of human antibody demonstrating the highest homology are selected and cloned followed by modifying the base sequence of the CDR by site-directed mutagenesis using the megaprimer method. In cases in which antigen cannot be bound specifically following humanization of the amino acid sequence that composes the framework region, the amino acids of a portion of the framework region may be converted from human amino acids to rat amino acids.

CDRs comprising an amino acid sequence having one or two amino acid deletions, substitutions or additions in the original sequence, and CDRs comprising an amino acid sequence having identity of X % or more with the original sequence can be produced using a known method such as site-specific mutagenesis, random mutagenesis, chain shuffling or CDR walking.

According to these methods, CDRs having a higher degree of affinity maturation are well known among persons with ordinary skill in the art to be obtained by presenting antibodies or antibody fragments having various mutations in the CDR thereof on a phage surface by phage display followed by screening using antigen (see, for example, Wu, et al., PNAS, 95, 6037-6042 (1998); Schier, R., et al., J. Mol. Biol., 263, 551-567 (1996); Schier, R., et al., J. Mol. Biol., 255, 28-43 (1996); Yang, W. P., et al., J. Mol. Biol., 254, 392-403 (1995)). The present invention also includes an antibody containing the CDR subjected to affinity maturation using such methods.

Examples of other antibody production methods include the Adlib method, by which an antibody-producing line is acquired from a chicken B cell-derived DT40 cell line treated with trichostatin A (Seo, H., et al., Nat. Biotechnol., 6, 731-736, 2002), and a method involving immunizing KM mice introduced with human antibody gene following destruction of mouse antibody gene to produce human antibody (Itoh, K., et al., Jpn. J. Cancer Res., 92, 1313-1321, 2001; Koide, A., et al., J. Mol. Biol., 284, 1141-1151, 1998), and these methods can be applied to production of the antibody according to the present invention.

An antigen-binding fragment of the anti-TMEM-180 antibody according to the present invention may be expressed according to the above-mentioned methods using DNA encoding the fragment, or by fragmenting the full-length antibody by treating with an enzyme such as papain or pepsin.

Antibodies that compete with a certain antibody for binding to an antigen can be obtained by a competition assay which is well-known in the art. For example, if an antibody can block the binding of the desired antibody by at least 20%, preferably at least 20 to 50%, more preferably at least 50%, the antibody can be an antibody that compete for binding to the same antigen. A competing antibody can be confirmed by a cross-blocking assay, preferably a competitive ELISA assay. In a cross-blocking assay, for example, a microtiter plate is coated with an antigen and then a candidate competing antibody is added to the plate, which is followed by incubation to form binding between the antigen and the candidate antibody. Then, the desired antibody, which has been labeled, is further added to the plate, and the plate is washed. Whether the antibody competes or not can be determined by measuring the amount of the bound desired antibody. The amount of the labels left in the well should decrease when the antibody competes.

The anti-TMEM-180 antibody according to the present invention can vary in terms of amino acid sequence, molecular weight, isoelectric point, presence or absence of oligosaccharide or form and the like according to the production method or purification method. However, the resulting antibody is included in the present invention provided it has a function that is equivalent to that of the antibody of the present invention. For example, in the case the antibody of the present invention has been expressed in E. coli or other prokaryotic cells, a methionine residue is added to the N-terminal of the amino acid sequence of the original antibody. This antibody is also included in the present invention.

In the case the anti-TMEM-180 antibody according to the present invention is an antibody having an N-linked oligosaccharide in which fucose is not bound to N-acetylglucosamine of the reducing end, the antibody can be produced in accordance with a known method or method complying therewith. Methods for producing this antibody are described in, for example, WO 2002/031140 and Japanese Patent Application Publication No. 2009-225781, and the disclosure thereof is incorporated in the present description in its entirety by reference.

More specifically, the anti-TMEM-180 antibody according to the present invention can be obtained, for example, by transforming cells deficient in or lacking enzyme activity involved in synthesis of GDP-fucose or α-1,6-fucosyl transferase activity using a vector containing DNA encoding the anti-TMEM-180 antibody according to the present invention, and then culturing the resulting transformant followed by purifying the target anti-TMEM-180 antibody.

Examples of enzymes involved in synthesis of GDP-fucose include GDP-mannose 4,6-dehydratase (GMP), GDP-keto-6-deoxymannose 3,5-epimerase, 4-reductase (Fx) and GDP-beta-L-fucose pyrophosphorylase (GFPP).

Here, although there are no particular limitations thereon, the cells are preferably mammalian cells, and for example, CHO cells deficient in or lacking the above-mentioned enzyme activity can be used.

Although there are cases in which an antibody composition obtained according to the above-mentioned method may contain antibody in which fucose is bound to N-acetylglucosamine on the reducing end, the proportion of antibody in which fucose is bound in this manner is 20% by weight or less, preferably 10% by weight or less, more preferably 5% by weight or less, and most preferably 3% by weight or less of total antibody weight.

In addition, antibody having an N-linked oligosaccharide in which fucose is not bound to N-acetylglucosamine on the reducing end can be obtained by introducing a vector containing DNA encoding the anti-TMEM-180 antibody according to the present invention into insect eggs, allowing the eggs to hatch and grow into insects, and then crossbreeding the insects as necessary to produce transgenic insects followed by extracting the anti-TMEM-180 antibody from the transgenic insects or secretions thereof. Silkworms can be used as the transgenic insects and in this case, antibody can be extracted from the cocoon.

Although there are also cases in which an antibody composition obtained according to this method may contain antibody in which fucose is bound to N-acetylglucosamine of the reducing end, the proportion antibody in which fucose is bound in this manner is 20% by weight or less, preferably 10% by weight or less, more preferably 5% by weight or less, and most preferably 3% by weight or less of total antibody weight.

(Activity of Antibody of Present Invention)

The mechanism of the efficacy of an antibody drug is based on two types of biological activity associated with the antibody. The first is binding activity specific to a target antigen in which the function of a target antigen molecule is neutralized as a result of binding thereto. Neutralization of the function of a target antigen molecule is demonstrated via the Fab region.

The other type of biological activity is antibody biological activity referred to as effector activity. Effector activity is demonstrated via the antibody Fc region in the form of, for example, antibody-dependent cellular cytotoxicity (ADCC), complement-dependent cytotoxicity (CDC) or direction induction of apoptosis.

Activity of the anti-TMEM-180 antibody according to the present invention can be measured using the methods indicated below.

(1) Binding Activity

Antibody binding activity can be measured using a known method such as enzyme-linked immunosorbent assay (ELISA), enzyme immunoassay (EIA), radioimmunoassay (RIA), fluorescent antibody technique or FACS.

(2) ADCC Activity

ADCC activity refers to activity that causes damage to a target cell when the antibody of the present invention has bound to an antigen on the surface of a target cell and an Fcγ receptor-retaining cell (effector cell) binds to the Fc moiety thereof via an Fcγ receptor.

ADCC activity can be determined by mixing a target cell expressing TMEM-180, an effector cell and the antibody of the present invention and measuring the degree of ADCC. Examples of effector cells that can be used include mouse spleen cells and monocytes isolated from human peripheral blood or bone marrow. TMEM-180-positive colon cancer mucosal cells, for example, can be used as target cells. The target cells are preliminarily labeled with a label such as ⁵¹Cr and the antibody of the present invention is then added thereto and incubated, followed by adding a suitable ratio of effector cells to the target cells and incubating. Following incubation, the supernatant is collected and ADCC activity can be measured by counting the level of the above-mentioned label in the supernatant.

(3) CDC Activity

CDC activity refers to cytotoxic activity involving complement.

CDC activity can be measured by using complement instead of effector cells in a test of ADCC activity.

(4) Tumor Growth Inhibitory Activity

Tumor growth inhibitory activity can be measured using tumor model animals. For example, the antibody of the present invention is administered after having transplanted a tumor beneath the skin of a mouse. Tumor growth inhibitory effect can be measured by comparing the volume of tumor tissue between a non-dose group and dose group.

Furthermore, the tumor growth inhibitory activity of the present invention may be that occurring as a result of inhibiting the growth of individual cells or that occurring as a result of inducting cell death.

(Testing Method and Testing Kit)

Cancers to be detected by the testing method of the present invention or with the testing kit of the present invention may include, for example, solid cancers, for example, colon cancer, brain tumor, gastric cancer, pancreas cancer, breast cancer, prostate cancer, kidney cancer, and bladder cancer. As was previously described, TMEM-180 is expressed in specific cancer cells, while TMEM-180 is not expressed in normal tissues and tissues of healthy subjects. Thus, the cancer testing method according to the present invention includes a step of measuring the amount of TMEM-180 in a sample collected from a subject.

In the present description, the sample collected from a subject can be any sample suitable for cancer testing, and can be suitably determined and collected according to the type of cancer by a person with ordinary skill in the art. Examples of samples include, but are not limited to, serum, plasma, whole blood, urine, stool, coelomic fluid and tissue. In the case of testing for colon cancer, the sample may be tissue collected from the subject with a colon endoscope or the like, or mucosal cells contained in washings following colonoscopy.

Carcinoembryonic antigen (CEA) has conventionally been widely used as a marker for colon cancer. Although plasma CEA levels decrease due to treatment such as surgical excision of the cancer, since they increase again accompanying recurrence or metastasis, measurement of CEA level is used to monitor progress following treatment. In cases in which CEA level has been observed to rise again, follow-up examinations involving abdominal ultrasonography and abdominal CT are required. However, since only 45% of colon cancer patients demonstrate an increase in CEA level, patients having cancer of a type that does not exhibit an increase in CEA level must undergo several rounds of follow-up examinations in order to monitor their progress.

In contrast, plasma TMEM-180 levels increase even in patients not exhibiting increases in CEA levels and are lower after surgery than before surgery as is indicated in the examples to be subsequently described. Moreover, plasma TMEM-180 levels are also observed to temporarily decrease after surgery and then exhibit a significant increase at the time of recurrence. This increase has been shown to be observed particularly in patients who do not exhibit an increase in CEA levels at the time of recurrence. Thus, this is considered to be an extremely useful marker capable of being used in a wide range of cancer patients.

The cancer testing method according to the present invention may be used for cancer diagnosis or may be used to confirm recurrence or metastasis when monitoring progress following surgery or other treatment.

In the present description, the “step for measuring the amount of TMEM-180 in a sample” includes not only a step of quantifying the amount of TMEM-180, but also includes a step of detecting for the presence or absence of TMEM-180, a step of determining a change in the amount of TMEM-180, and a step of comparing with the amount of TMEM-180 present in another sample.

Cancers (or the existence of cancers) can be detected by detecting exosomes expressing TMEM-180 on the surface, because TMEM-180 expresses on the membranes of the exosomes contained in the body fluid sample (for example, serum, plasma, whole blood, urine, and celomic fluid) obtained from a subject. Then, when exosomes expressing TMEM-180 on the surfaces are detected, it is indicated that the existence of cancer in the body of the subject is detected. The “step of measuring the amount of TMEM-180 in a sample” of above may comprise a step of detecting the existence or absence or the amount of exosomes expressing TMEM-180 on the surface in the body fluid sample (for example, serum, plasma, whole blood, urine, or celomic fluid) and a step of determining that cancer is detected when the amount of the exosomes detected are higher than a prescribed value. The prescribed value can be determined by those skilled in the art based on a false positive rate and a false negative rate. Namely, in order to decrease the false negative rate, the prescribed value needs to be increased, and in order to decrease the false positive rate, the prescribed value needs to be decreased.

The prescribed value may include, for example, but not limited to, the amount of the exosomes in the body fluid sample from a healthy subject. The prescribed value may also include, for example, but not limited to, the average of the amounts of the exosomes in the body fluids from a plurality of healthy subjects. When comparing to the amount of the exosomes in the body fluids from a plurality of healthy subjects, the prescribed value may be the average value of the amounts, the maximum value among the amounts, the third quartile value of the amounts, or a value between two values from these values, or a value larger than the value. Further, the prescribed value may be, for example, but not limited to, a value more than the average value, the maximum value, or the third quartile value, or a value selected between two values from these values, a value more than twice of the value, a value more than triple of the value, a value more than four times of the value, or a value more than five times of the value. For example, the prescribed value may be the average value of the amounts of the exosomes in the body fluid samples from a plurality of healthy subjects. For example, the prescribed value may be the maximum value among the amounts of the exosomes in the body fluid samples from a plurality of healthy subjects. For example, the prescribed value may be a value more than the average value of the amounts of the exosomes in the body fluid samples from a plurality of healthy subjects, a value more than twice of the average value, a value more than triple of the average value, a value more than four times of the average value, or a value more than five times of the average value, or a value from twice to five times of the average value or a value from triple to five times of the average value. Cancers to be detected in the invention may include, for example, solid cancers, for example, colon cancer, brain tumor, gastric cancer, pancreas cancer, breast cancer, prostate cancer, kidney cancer, and bladder cancer. The number of the healthy subjects which should be referred in order to determine the prescribed value may be, for example, but not limited to, 5 to 100 subjects.

Detecting exosomes expressing TMEM-180 on the surface can be performed by detecting TMEM-180 after purification of exosomes, or by detecting exosomes in a purified product after purification of the substance containing TMEM-180. Namely, the present invention provides a method for detecting exosomes, comprising purifying exosomes expressing TMEM-180 on the surface from a body fluid sample, and detecting the exosomes expressing TMEM-180 on the surface, thereby determining that exosomes expressing TMEM-180 on the surface exist in the body fluid sample, provided that anti-TMEM-180 antibody is used in the purification or the detection, or both.

Purifying or detecting exosomes can be performed by well-known techniques, for example, but not limited to, using an antibody binding to exosomes (e.g., anti-CD9 antibody, anti-CD63 antibody, or anti-CD81 antibody). Detecting TMEM-180 or purifying substance containing TMEM-180 can be performed by using the anti-TMEM-180 antibody of the present invention. An antibody to be used in the detection may be an antibody which has been detectably labelled with a label such as an enzyme including peroxidase and alkaline phosphatase, a radio isotope, a fluorescent material, and a luminescent material. Detecting such an antibody can be performed by a technique well-known by those skilled in the art. The amount of exosomes and the above-mentioned prescribed value can be determined based on the measured values by these detecting methods or a standardized value therefrom. Exosomes can be purified with an anti-TMEM-180 antibody and detected with another anti-TMEM-180 antibody.

For example, exosomes expressing TMEM-180 on the surface can be immunoprecipitated from a sample with beads attaching the anti-TMEM-180 antibody of the present invention, and then can be detected with an antibody that binds to exosomes (e.g., anti-CD9 antibody, anti-CD63 antibody, or anti-CD81 antibody) in the purified TMEM-180 fraction. For example, exosomes expressing TMEM-180 on the surface can also be immunoprecipitated from a sample with beads attaching an antibody that binds to exosomes (e.g., anti-CD9 antibody, anti-CD63 antibody, or anti-CD81 antibody), and then can be detected with the anti-TMEM-180 antibody of the present invention in the purified TMEM-180 fraction.

Any anti-TMEM-180 antibody can be used as the antibody of the present invention in purifying or detecting exosomes, and, for example, the 1361 antibody or the 1361-5 antibody, or an antibody that compete with these antibodies for binding to TMEM-180 can be used.

The amount of TMEM-180 in a sample can be measured using any method used to measure the amount of a specific protein in a sample, and examples thereof include, but are not limited to, immunoassay (including agglutination and turbidimetry), western blotting and surface plasmon resonance (SPR). Among these, immunoassay using an anti-TMEM-180 antibody or an antigen-binding fragment thereof is both easy and useful.

Since TMEM-180 is a membrane protein, the amount thereof may be measured after having solubilized the membrane protein by treating with a commercially available cell lysis buffer, nonionic surfactant, membrane protein solubilizing reagent or the like.

Immunoassay uses an anti-TMEM-180 antibody labeled to allow subsequent detection and/or an antibody to the anti-TMEM-180 antibody labeled to allow subsequent detection (secondary antibody). Antibody labeling methods are classified into enzyme immunoassay (EIA or ELISA), radioimmunoassay (RIA), fluorescent immunoassay (FIA), fluorescence polarization immunoassay (FPIA), chemiluminescence immunoassay (CLIA), fluorescence enzyme immunoassay (FLEIA), chemiluminescence enzyme immunoassay (CLEIA), electrochemiluminescence immunoassay (ECLIA) and the like, and any of these methods can be used in the method of the present invention.

An antibody labeled with an enzyme such as peroxidase or alkaline phosphatase is used in the ELISA method, an antibody labeled with a radioactive substance such as ¹²⁵I, ¹³¹I, ³⁵S or ³H is used in the RIA method, an antibody labeled with a fluorescent substance such as fluorescein isocyanate, rhodamine, dansyl chloride, phycoerythrin, tetramethylrhodamine isothiocyanate or near-infrared fluorescent material is used in the FPIA method, and an antibody labeled with a luminescent substance such as luciferase, luciferin or aequorin is used in the CLIA method. Antibodies labeled with other substances in the manner of nanoparticles such as metal colloids or quantum dots can also be detected.

In addition, when using an immunoassay, the anti-TMEM-180 antibody can also be detected by labeling with biotin and then binding with avidin or streptavidin labeled with an enzyme and the like.

Among these immunoassays, the ELISA method using an enzyme label enables antigen to be measured both easily and quickly.

The ELISA method includes a competitive method and a sandwich method. In the competitive method, an anti-TMEM-180 antibody or an antigen-binding fragment thereof is immobilized on a microplate or other solid-phase support, and a sample and enzyme-labeled TMEM-180 are added thereto followed by allowing an antigen-antibody reaction to occur. After having washed the support, the antigen-antibody complex is allowed to react with the enzyme substrate resulting in the generation of color followed by measurement of optical absorbance. Since coloring is lighter if a large amount of TMEM-180 is contained in the sample and darker if only a small amount thereof is contained in the sample, the amount of TMEM-180 can be determined using a calibration curve.

In the sandwich method, an anti-TMEM-180 antibody or an antigen-binding fragment thereof is immobilized on a solid-phase support, and after adding a sample and allowing to react, an enzyme-labeled anti-TMEM-180 antibody that recognizes a different epitope is further added and allowed to react. After washing, the resulting complex is allowed to react with the enzyme substrate to generate color followed by measurement of optical absorbance to determine the amount of TMEM-180. In the sandwich method, after having reacted TMEM-180 in a sample with antibody immobilized on a solid-phase support, an unlabeled antibody (primary antibody) may be added followed by enzyme-labeling an antibody to this unlabeled antibody (secondary antibody) and further adding thereto.

Examples of enzyme substrates that can be used in the case the enzyme is peroxidase include 3,3′-diaminobenzidine (DAB), 3,3′,5,5′-tetramethylbenzidine (TMB) and o-phenylenediamine (OPD), while examples of substrates in the case the enzyme is alkaline phosphatase include p-nitropheny phosphate (NPP).

Alternatively, TMEM-180 antigen in a sample may be immobilized directly on a microtiter plate or other solid-phase support, and after carrying out the required blocking, an unlabeled antibody (primary antibody) is added followed by enzyme-labeling and further adding an antibody to this unlabeled antibody (secondary antibody).

In the present description, there are no particular limitations on the “solid-phase support” provided it is a support that is capable of immobilizing antibody thereon, and examples thereof include microtiter plates made of glass, metal, resin or the like, substrates, beads, nitrocellulose membranes, nylon membranes and PVDF membranes, and a target substance can be immobilized on these solid-phase supports in accordance with known methods.

In addition, among these immunoassays, agglutination methods are preferable since they enable a trace amount of protein to be detected easily. Examples of agglutination methods include latex agglutination that uses latex particles bound to antibody.

When the anti-TMEM-180 antibody is allowed to bind to the latex particles and then mixed with a suitably treated sample, antibody-bound latex particles end up agglutinating if TMEM-180 is present. Therefore, antigen concentration can be determined by quantifying the amount of agglomerate by irradiating the sample with near-infrared light and measuring optical absorbance (turbidimetry) or measuring scattered light (nephelometry).

The previously described anti-TMEM-180 antibody, or antigen-binding fragment thereof, having the previously listed CDR sequences may be used for the anti-TMEM-180 antibody or antigen-binding fragment thereof.

The cancer testing kit according to the present invention is a kit for testing for cancer using the above-mentioned testing method, and contains an anti-TMEM-180 antibody or antigen-binding fragment thereof. In addition to the anti-TMEM-180 antibody or antigen-binding fragment thereof, the cancer testing kit according to the present invention may also contain reagents and apparatuses required to measure the amount of TMEM-180 in a sample by immunoassay.

One aspect of a testing kit is that for measuring TMEM-180 according to the sandwich method, and contains a microtiter plate, anti-TMEM-180 capture antibody or antigen-binding fragment thereof, an anti-TMEM-180 antibody or antigen-binding fragment thereof labeled with alkaline phosphatase or peroxidase, and alkaline phosphatase substrate (such as NPP) or peroxidase substrate (such as DAB, TMB or OPD).

The capture antibody and labeled antibody recognize different epitopes.

In this type of kit, the capture antibody is first immobilized on a microtiter plate followed by suitably diluting a sample and adding thereto, incubating, removing the sample and washing. Next, the labeled antibody is added followed by incubating and adding substrate to develop color. The amount of TMEM-180 can be determined by measuring the degree of coloring using a microtiter plate reader and the like.

Another aspect of the testing kit is for measuring TMEM-180 according to the sandwich method using secondary antibody, and contains a microtiter plate, anti-TMEM-180 capture antibody, primary antibody in the form of anti-TMEM-180 antibody, secondary antibody in the form of alkaline phosphatase- or peroxidase-labeled anti-TMEM-180 antibody, and alkaline phosphatase substrate (such as NPP) or peroxidase substrate (such as DAB, TMB or OPD).

The capture antibody and primary antibody recognize different epitopes.

In this type of kit, the capture antibody is first immobilized on a microtiter plate followed by suitably diluting a sample and adding thereto, incubating, removing the sample and washing. Continuing, the primary antibody is added followed by incubating and washing, after which the enzyme-labeled secondary antibody is added and incubated followed by adding the substrate to develop color. The amount of TMEM-180 can be determined by measuring the degree of coloring using a microtiter plate reader and the like. The use of secondary antibody makes it possible to amplify the reaction and enhance detection sensitivity.

The testing kit may further contain a required buffer solution, enzyme reaction stopping solution or microplate reader and the like.

The labeled antibody is not limited to an enzyme-labeled antibody, but rather may also be an antibody labeled with a radioactive substance (such as ²⁵I, ¹³¹I, ³⁵S or ³H), fluorescent substance (such as fluorescein isocyanate, rhodamine, dansyl chloride, phycoerythrin, tetramethylrhodamine isothiocyanate or near-infrared fluorescent material), luminescent substance (such as luciferase, luciferin or aequorin), nanoparticles (such as metal colloids or quantum dots) or the like. In addition, biotinated antibody can be used for the labeled antibody and labeled avidin or streptavidin can be added to the kit.

Still another aspect of the testing kit is a testing kit used to measure the amount of TMEM-180 by latex agglutination. This kit contains anti-TMEM-180 antibody-sensitized latex, and agglomerates are quantified by an optical method after mixing with a sample and an anti-TMEM-180 antibody. An agglutination reaction plate for visualizing the agglutination reaction is also preferably contained in the kit.

Disclosures of all patent documents and non-patent documents cited in the present description are incorporated in the present description in their entirety by reference.

### EXAMPLES

Although the following provides a detailed explanation of the present invention based on examples thereof, the present invention is not limited thereto. A person with ordinary skill in the art would be able to modify the present invention in various forms without deviating from the significance of the present invention, and all such modifications are included within the scope of the present invention.

1. Identification of TMEM-180 Molecule

1) DNA Microarray Analysis

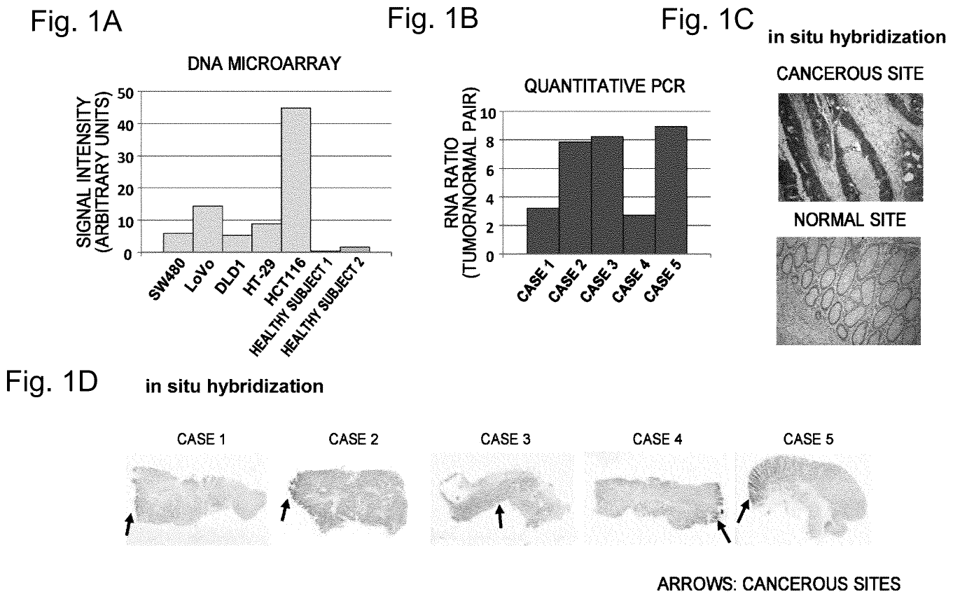

10 μg aliquots of mRNA were used that were respectively derived from five types of human colon cancer cell lines (HT29, SW480, LOVO, HCT116 and DLD-1) and two types of free colon cells derived from healthy human subjects. Biotin-labeled cRNA was synthesized after obtaining double-stranded cDNA from RNA in accordance with the instructions of the manufacturer (Affymetrix, Inc.). Following fragmentation, the fragments were hybridized with the GeneChip Human Genome U133 Plus 2.0 Array (Affymetrix, Inc.). The hybrids were scanned using the GeneChip Scanner 3000 7G (Affymetrix, Inc.). CEL data was acquired and then subjected to statistical processing to calculate the signal value of each sample. TMEM-180 was selected as a colon cancer cell-specific surface marker for which expression was observed in the five types of human colon cancer cell lines but not observed in the two types of healthy subject colon cells (FIG. 1A).

2) Quantitative PCR

Colon cancer cell tissue sample cDNA (Clontech Laboratories, Inc.) was analyzed with the ABI 7500Fast analyzer (Applied Biosystems) using adjacent normal colon tissue as a negative control for the colon cancer surgical specimens of five patients. 10 μL of 2× TaqMan Fast Universal PCR Master Mix (Applied Biosystems) and 1 μL of 20× TaqMan Gene Expression Assay (Applied Biosystems) were used in 20 μL of reaction solution. In addition, quantitative PCR was carried out with a fast run under conditions of 40 cycles of AmpliTaq Gold Enzyme activation at 95° C. and 20 seconds per cycle, denaturation at 95° C. for 3 seconds, and annealing/extending for 30 seconds at 62° C. The results were analyzed with 7500 Fast System SDS Software Version 1.3. The results of calculating the ratio of the amount of RNA in colon cancer tissue to the amount of RNA in normal tissue are shown in FIG. 1B for each case. In each of the cases, TMEM-180 was confirmed to be strongly expressed in colon cancer tissue as compared with normal colon tissue.

3) In Situ Hybridization

A colon cancer tissue paraffin section (Genostaff Co., Ltd.) was subjected to dewaxing treatment with xylene and then hydrated with ethanol and PBS. The section was then fixed for 15 minutes with 4% para-formaldehyde in PBS. After treating with PBS containing 7 μg/mL of Proteinase K (F. Hoffmann-La Roche Ltd.), the section was again fixed with 4% para-formaldehyde in PBS. The section was acetylated with 0.1 M Tris-HCl (pH 8.0) containing 0.25% acetic anhydride. After washing with PBS, the section was dehydrated with ethanol. The section was then subjected to a hybridization reaction with 300 ng/mL of an RNA probe of digoxigenin-labeled TMEM-180 (475 bp sequence from the 1314th nucleotide to the 1789th nucleotide of GeneBank Accession No. NM_024789, Genostaff Co., Ltd.) for 16 hours at 60° C. After washing, the section was treated with 50 μg/mL of RNaseA, 10 mM Tris-HCl (pH 8.0), 0.1 M NaCl and 1 mM EDTA. After washing again, the section was allowed to react using 0.5% blocking reaction solution (F. Hoffmann-La Roche Ltd.) followed by reacting for 1 hour in blocking reaction solution containing 20% heat-treated sheep serum (Sigma-Aldrich Co. LLC.). AP-labeled anti-DIG antibody (F. Hoffmann-La Roche Ltd.) was then added and allowed to react for 2 hours at room temperature. After washing, color was developed in NBT/NCIP solution (F. Hoffmann-La Roche Ltd.). The results of enclosure and microscopic observation are shown in FIGS. 1C and 1D. TMEM-180 was confirmed to be positive in all five types of the colon cancer tissue and negative in normal colon mucosa.

2. Expression Analysis of TMEM-180 in Normal Tissue

A search was made for TMEM-180 using the public database, PaxDB, in the form of a comprehensive absolute protein abundance database (http://pax-db.org/#!home, which measures the expression level of each protein by analyzing unique peptides by LC/MS/MS) to investigate measured values in each type of normal tissue. Other than TMEM-180, expression of the following molecules was also investigated as controls.
β-actin ((SACT) (housekeeping molecule)
Glyceraldehyde 3-phosphate dehydrogenase (GAPDH) (housekeeping molecule)
Epidermal growth factor receptor (EGFR) (target molecule of antibody drug)
HE R2 (target molecule of antibody drug)
Carcinoembryonic antigen (CEA) (typical tumor marker molecule)

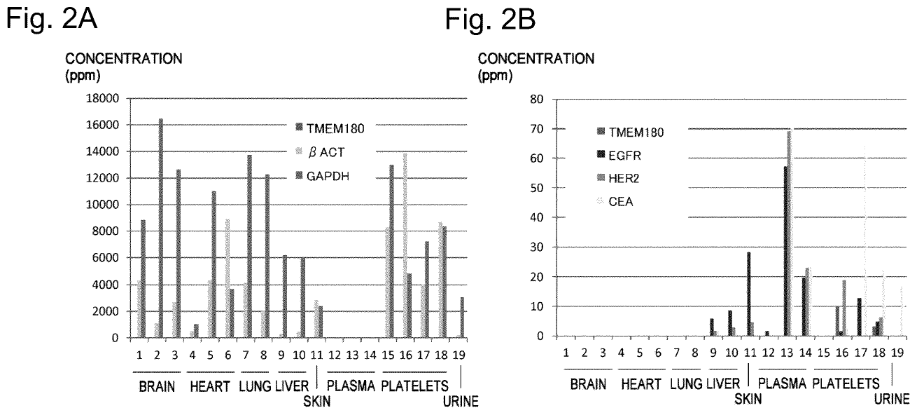

The results of entering the data into an Excel (2010, Microsoft Corporation) file and generating graphs are shown in FIG. 2. Although TMEM-180 is rarely expressed normally (FIG. 2A) and was observed to be only slightly expressed in platelets when sensitivity was increased, expression was much lower than the conventional antibody drug target molecules of EGFR and HER2 (FIG. 2B), thereby confirming that TMEM-180 is specifically expressed in cancer tissue.

3. Antibody Production and FACS on Various Types of Cancer Cells

1) Antigen Production

[PCR Reaction]

PCT amplification was carried out on a tag sequence incorporated in pET21 b using the primers indicated below.

PCR enzyme used: PrimeStar HS DNA polymerase (Takara Bio Inc., R010A)

Primer sequences for producing immunizing
antigen 1:
(i)
(SEQ ID NO: 18)
cagacctgcacctgaacgtggttgagagctgaggaattgacggtcactga
gggactgtaatgctgcacttcgc
(ii)
(SEQ ID NO: 19)
caaccacgttcaggtgcaggtctgtcttcacgtgctgttgtactggctcg
tgttcaagagttcaaactggaggacctg
(iii)
(SEQ ID NO: 20)
gagatatacatatgtcggaggtgactcgtagtc
(iv)
(SEQ ID NO: 21)
tggtgctcgagaataggctgaacatcaaatg
Primer sequences for producing immunizing
antigen 2:
(i)
(SEQ ID NO: 22)
gcgaagtgcagcattacagtccgatcatctgagtctgctgtgcctcttca
ttgc
(ii)
(SEQ ID NO: 23)
caggtcctccagtttgaactcagaagctgcttgcttacgatgattcagca
ccaggtcctcgtctac
(iii)
(SEQ ID NO: 24)
gagatatacatatgtcggaggtgactcgtagtc
(iv)
(SEQ ID NO: 25)
tggtgctcgagaataggctgaacatcaaatg

[Restriction Enzyme Treatment]

NdeI (Takara Bio Inc.) and XhoI (Takara Bio Inc.) were reacted for 2 hours with expression vector pET21 b and the PCR products in accordance with the manufacturer's protocol, and carrying out 1% agarose gel electrophoresis, the products were purified using the Promega Wizard SV Gal and PCR Clean-up System kit.

[Ligation Reaction]

A vector and insert were reacted for 30 minutes using Ligation High (Toyobo Co., Ltd.).

[Transformation]

Transformation was carried out using Competent High DH5α (Toyobo Co., Ltd.) followed by culturing in plates containing LB medium (50 μg/mL).

[Gene Confirmation]

The plasmid was extracted from the transformed E. coli and the sequence was analyzed to confirm the target sequence.

[Transformation (E. coli)]

BL21(DE3) was transformed with the plasmid inserted with the target sequence.

[Culturing]

The transformed E. coli was inoculated into 10 mL of LB medium and cultured for 16 hours at 37° C. followed by transferring the medium to 1 L of LB medium and culturing at 37° C. IPTG was added to a final concentration of 1 mM when the OD value at 600 nm reached 0.6 followed by additionally culturing for 4 hours.

[Purification]

Sediment of the disrupted E. coli was suspended in 50 mM Tris-HCl, 500 mM NaCl and 6 M GdnHCl followed by recovering supernatant from the sample after shaking for 16 hours and purifying with a nickel column.

[Refolding]

Since the target antigen had been denatured as a result of dissolving with 6 M GdnHCl, refolding (protein unwinding) was carried out by dialysis.

Refolding was carried out according to the following steps under the conditions indicated below.
(1) 50 mM Tris-HCl, pH 8.5, 500 mM NaCl, 1 mM EDTA, 1 mM DTT, 3 M GdnHCl, 6 hours
(2) 50 mM Tris-HCl, pH 8.5, 500 mM NaCl, 1 mM EDTA, 1 mM DTT, 2 M GdnHCl, 6 hours
(3) 50 mM Tris-HCl, pH 8.5, 500 mM NaCl, 1 mM EDTA, 1 mM DTT, 1 M GdnHCl, 12 hours
(4) 50 mM Tris-HCl, pH 8.5, 500 mM NaCl, 1 mM EDTA, 1 mM DTT, 0.5 M GdnHCl, 12 hours
(5) 50 mM Tris-HCl, pH 8.5, 150 mM NaCl, 50 mM L-Arg, 6 hours
(6) 50 mM Tris-HCl, pH 8.5, 150 mM NaCl, 50 mM L-Arg, 6 hours

The dialysis solution of the above-mentioned sample was replaced with 50 mM phosphate buffer (pH 8.0) and 500 mM NaCl using an ultrafiltration membrane (Amicon-Ultra 10K).

[Measurement of CD]

Helix content was confirmed to approach the theoretical value using a circular dichroic disperser (JASCO Corporation, J-725) in order to confirm whether the refolded immunizing antigen retained its steric structure.

2) Antibody Production

An emulsion prepared by mixing immunizing antigen 1 or immunizing antigen 2, diluted to 100 jig/mL with PBS, with Freund's Complete Adjuvant in a 1:1 ratio was administered in 100 μL aliquots at both sides of the base of the caudal vein in rats (Japan SLC, Inc., Wistar, females, age 6 to 8 weeks). Blood was collected from the caudal vein at 12 to 13 days after immunization and serum antibody titers were evaluated by ELISA using the prepared antiserum and immunizing antigen for the solid phase or by flow cytometry using DLD-1 cells and K562 cells to select individuals for use in cell fusion. Iliac lymph nodes, inguinal lymph nodes, axillary lymph nodes and popliteal lymph nodes were dissected from individuals selected at 14 days after immunization, and lymph node cells and mouse myeloma cells p3X63 were fused according to the PEG method. Culture supernatant was recovered 10 to 14 days after fusion and antibody-producing hybridoma cells that were positive for DLD-1 cells and negative for K562 cells were selected by flow cytometry. The selected antibody-producing hybridoma cells were single-cloned and established by limiting dilution. Antibody isotypes were identified by isotype-specific ELISA (Bethyl Laboratories).

The single-cloned clones were clone 98, clone 101, clone 212 (which are IgM antibodies), clone 129, clone 382, clone 1361 (which are IgG antibodies), clone 669, clone 699, clone 1052 and clone 1105 (which are IgM antibodies). An antibody obtainable from the clone having the clone number x may be referred to as “x antibody”.

3) Flow Cytometry

Cancer cells targeted for measurement were cultured in medium and then added to a V-bottom 96-well plate (Corning Incorporated) to a concentration of 1×10⁵ cells/well. The plate was centrifuged for 3 minutes at 440×g and 4° C. followed by removing the supernatant, adding antibody-producing hybridoma culture supernatant or antibody solution to the cell pellet at 50 μL/well and suspending therein. After allowing to react for 45 minutes on ice, the plate was washed three times with a mixture of 0.1% BSA, 2 mM EDTA and PBS at 200 μL/well. The supernatant was removed and secondary antibody was added to the cell pellet at 50 μL/well followed by suspending the cells therein. AlexaFluor 647 goat anti-rat IgG (H-L) (Life Technologies Corporation) was used for the secondary antibody after diluting 400-fold with a mixture of 0.1% BSA, 2 mM EDTA and PBS. After allowing to react for 45 minutes on ice, the plate was washed three times with a mixture of 0.1% BSA, 2 mM EDTA and PBS at 200 μL/well. After removing the supernatant, 50 ng/mL of propidium iodide, 0.1% BSA, 2 mM EDTA and PBS were added to the cell pellet at 250 μL/well followed by suspending therein. The cells stained in this manner were measured using a flow cytometer such as the Guava easyCyte 8HT (Merck Millipore Corporation) followed by analysis of the resulting data using FlowJO (Tomy Digital Biology Co., Ltd.). FACS analysis was carried out using colon cancer, brain tumor and hematologic cancer.

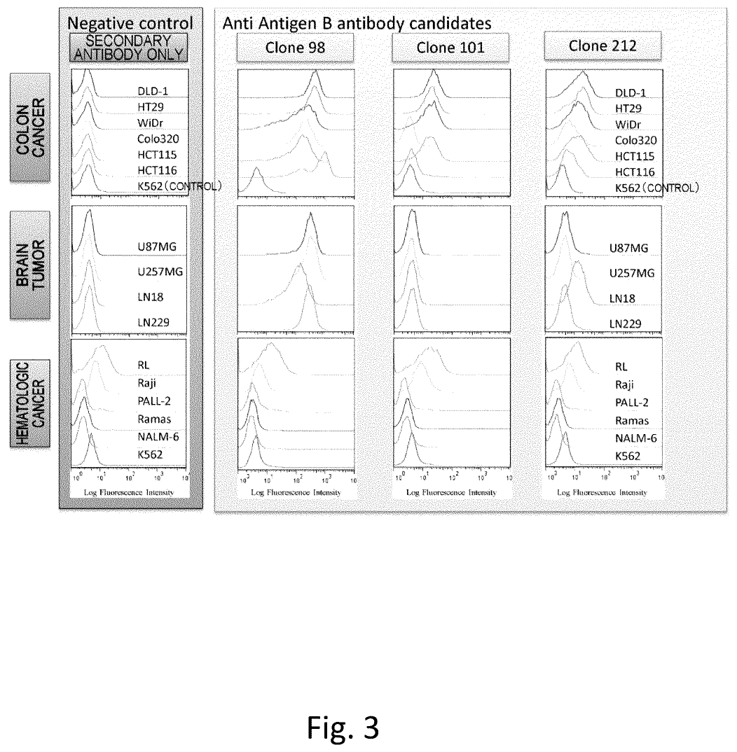

The results of FACS analysis using the acquired anti-TMEM-180 IgM antibody are shown in FIG. 3. Clone 98 was positive for all six types of colon cancers and all four types of brain tumors while clone 101 was positive for four types of colon cancers and negative for all of the brain tumors. Clone 212 was positive for all six types of colon cancers and was positive for only one type of brain tumor. All of the clones were negative for hematologic tumors.

4. Fluorescent Immunostaining of DLD-1 Colon Cancer Cells

DLD-1 cells were added to a 96-well plate (Corning Incorporated, CellBIND) to a concentration of 5×10³ cells/well and subjected to cell staining after culturing for 2 days. The cells were subjected to fixation and permeation treatment in the manner indicated below. After washing the cell culturing plate twice with PBS at 200 μL/well, the washings were removed followed by the addition of 4% para-formaldehyde-phosphate buffer (Wako Pure Chemical Industries, Ltd.) containing 0.1% Triton X-100 (Wako Pure Chemical Industries, Ltd.) at 100 μL/well while cooling with ice and fixing the cells for 10 minutes. After removing the fixation solution, the plate was washed once with PBS at 200 μL/well and once with a mixture of 1% FBS in PBS at 200 μL/well to obtain a cell-immobilized plate. The cells were stained in the manner indicated below. Namely, after removing medium from the non-immobilized cell culturing plate and removing washings from the cell-immobilized plate, cell-producing hybridoma culture supernatant or diluted antibody solution was added at 50 μL/well. After allowing to react for 1 hour on ice, the plate was washed once with PBS at 200 μL/well and twice with a mixture of 1% FBS in PBS at 200 μL/well. After removing the washings, a mixture of 5 μg/mL AlexaFluor 647 goat anti-rat IgG (H-L), 2 μg/mL Hoechst 33342 and 1% FBS/PBS was added at 50 μL/well. After allowing to react for 1 hour on ice, the plate was washed twice with PBS at 200 μL/well. PBS was then added at 50 μL/well followed by measuring with ArrayScan (Thermo Fisher Scientific Inc.).

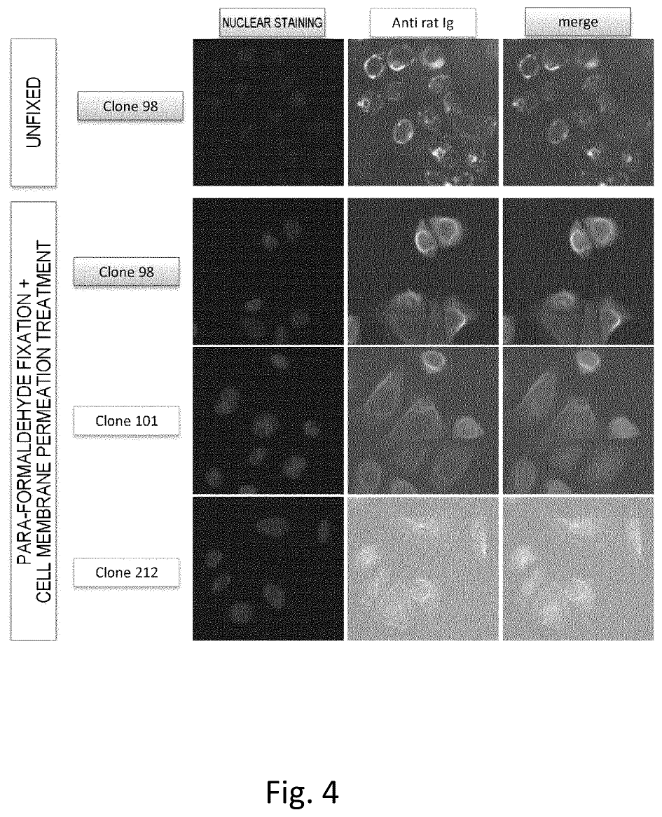

The results of fluorescent immunostaining using clones 98, 101 and 212 of anti-TMEM-180 IgM antibody are shown in FIG. 4. Clone 98 was stained primarily in the membrane. Clones 101 and 212 exhibited weaker fluorescence intensity than clone 98 and the cytoplasm was also stained.

5. FACS of DLD-1 Parent Line and TMEM-180 Knockout Line Using Anti-TME M-180 Antibody
1) Production of Knockout Cells

[Transfection]

2.5 μg of Sigma CRISPR/Cas9 System (HS0000468201) plasmid were diluted with 0.5 mL of Opti-MEM (Invitrogen Corporation) followed by the addition of 11 μL of Lipofectionamine LTX. After allowing to stand undisturbed for 30 minutes at room temperature, DLD-1 cells (6.26×10⁵ cells/well, 6-well plate (Corning Incorporated)) were added to the DNA-Lipofection preparation.

[Selection of GFP-Expressing Cells]

Cells cultured for 2 days after transfection were subjected to cell sorting using the FACSAria Cell Sorter (BD) to acquire only cells that express GFP.

[Cloning]

After culturing the GFP-expressing cells and confirming that GFP was no longer expressed, the cells were subjected to limiting dilution in a 96-well plate. Genome purification was carried out on those wells containing only a single cell colony using the PureLink Genomic DNA Mini Kit (Invitrogen Corporation) followed by determining knockout cells by confirming the target sequence.

2) Flow Cytometry

Flow cytometry was carried out in accordance with the procedure described in the above-mentioned section 3. Rat IgM antibody clone 98 used as primary antibody was used after diluting antibody-producing hybridoma culture supernatant to 1 μg/mL. The supernatant of a culture of hybridoma producing rat IgM antibody clone 365 was used as a negative control after similarly diluting to 1 μg/mL. Rat-human chimeric antibody clone 98 was used after diluting the culture supernatant of chimeric antibody constantly-expressing cells two-fold. Human tissue factor antibody clone hTF1849, rat anti-human EpCAM antibody clone B8-4, mouse anti-human CD44v6 antibody clone 2F10 (Medical & Biological Laboratories Co., Ltd.)

and mouse-human chimeric anti-EGFR antibody (trade name: Erbitux) were respectively used as positive controls after diluting to 1 μg/mL. A mixture of RPMI and 10% FBS was used to dilute each of the antibodies. Furthermore, antibody concentration in the hybridoma culture supernatant was measured by rat IgM-specific ELISA (Bethyl Laboratories). In addition, secondary antibody formed of any of AlexaFluor 647 goat anti-rat IgG (H+L), AlexaFluor 647 goat anti-human IgG (H+L) or AlexaFluor 647 goat anti-mouse IgG (H+L) (Life Technologies Corporation) was used corresponding to the origin of the primary antibody after diluting 400-fold with a mixture of 0.1% BSA, 2 mM EDTA and PBS.

3) Production of Human Chimeric Antibody

Total RNA was extracted from the hybridoma cell lines and cDNA of variable regions of antibody H chain and variable regions of antibody L chain was synthesized with the SMARTer RACE cDNA Amplification Kit (Takara Bio Inc.) using the 5′-end rapid amplification of cDNA ends (RACE) method.

The synthesized cDNA was subjected to amplification by PCR and cloned to pUC19 (Invitrogen Corporation). The variable regions of the H chain and variable regions of the L chain are shown in subsequently indicated Tables 3 to 22.

After amplifying the variable regions of the H chain and L chain by PCR, the variable region of the H chain was inserted into pQCxIP incorporating a constant region (Clontech Laboratories, Inc.) while the variable region of the L chain was inserted into pQCxIH incorporating a constant region (Clontech Laboratories, Inc.) to complete the expression vector. The expression vector was then transfected into 293T cells using Lipofectamine 2000 (Invitrogen Corporation). Human anti-TMEM-180 antibody constantly expressing cell line was subjected to drug selection using puromycin (Sigma-Aldrich Co. LLC.) at 101 μg/mL and hygromycin B (Invitrogen Corporation) at 1 mg/mL and established by acquiring a cell line resistant to both drugs. The established cell line was subjected to maintenance culturing in DMEM (sigma) with 10% FBS, 1% penicillin-streptomycin (Invitrogen Corporation), 10 μg/mL of puromycin and 1 mg/mL of hygromycin B.

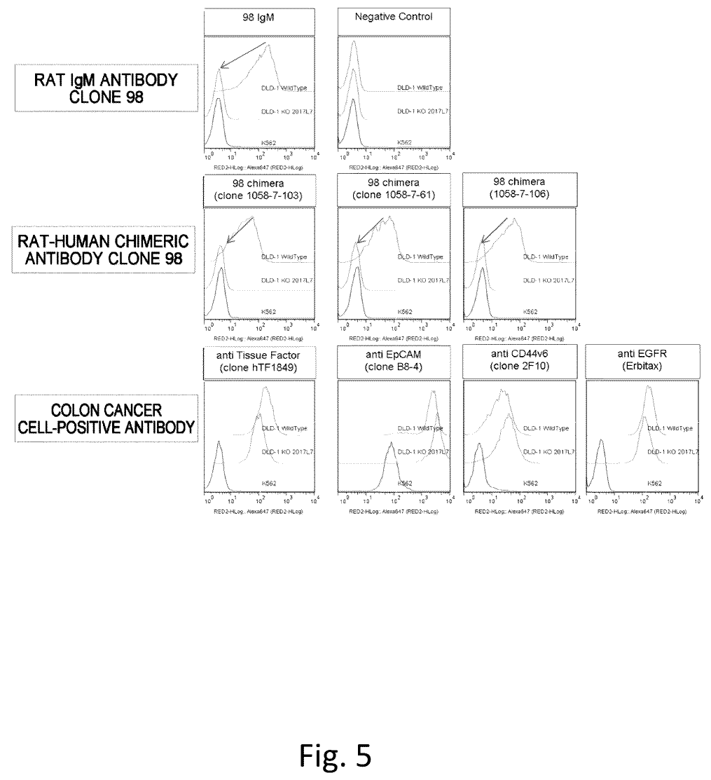

FAGS was carried out on the colon cancer DLD-1 parent line and the TMEM-180 knockout line using rat IgM antibody clone 98 and human chimeric IgG1 antibody. The results are shown in FIG. 5. The rat IgM antibody clone 98 and human chimeric IgG antibody both demonstrated a considerable left shift. There were no differences between the parent line and knockout line in FAGS using anti-tissue factor antibody, anti-EpCAM antibody, anti-CD44v6 antibody and anti-EGFR antibody as controls. This indicates that both rat IgM antibody clone 98 and human chimeric IgG1 antibody specifically recognize TMEM-180.

6. Immunostaining of Formalin-Fixed Section of Human Colon Cancer Surgical Specimens by Anti-TMEM-180 Antibody

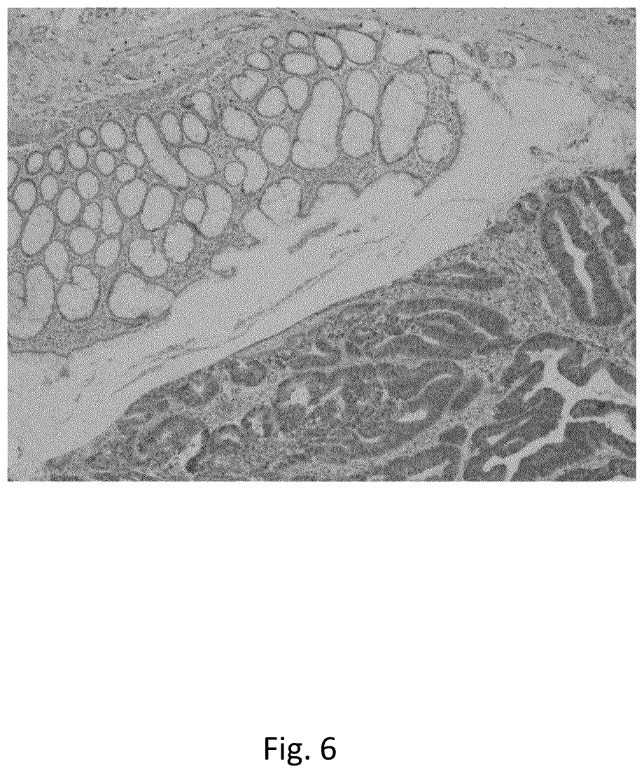

Anti-TMEM-180 IgM rat antibody (clone 98) was reacted with a colon cancer formalin-fixed section at 1 μg/mL. HRP-labeled anti-rat antibody was used as secondary antibody and the section was stained with DAB followed by post-staining with hematoxylin. The result is shown in FIG. 6. Colon cancer was shown to be stained specifically. Both the cell membrane and cytoplasm of the colon cancer cells were stained.

7. Cytocidal Effect of Anti-TMEM-180 IgM Antibody

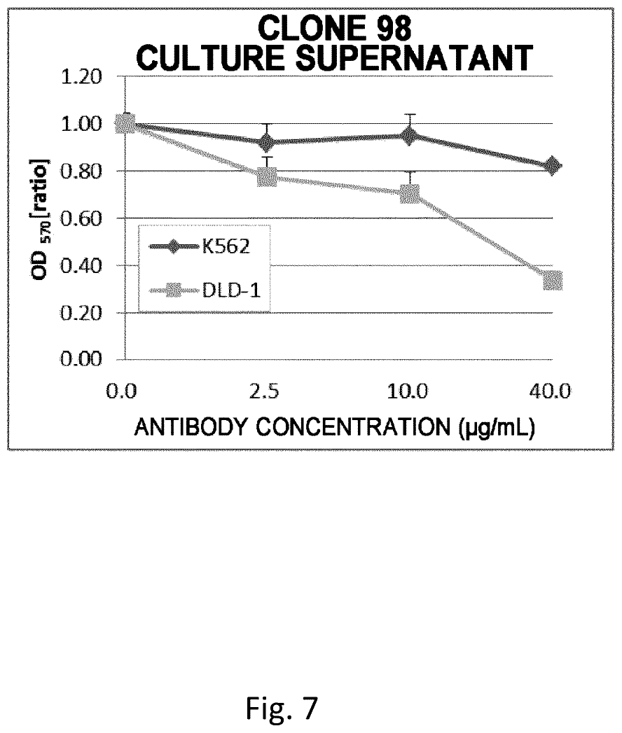

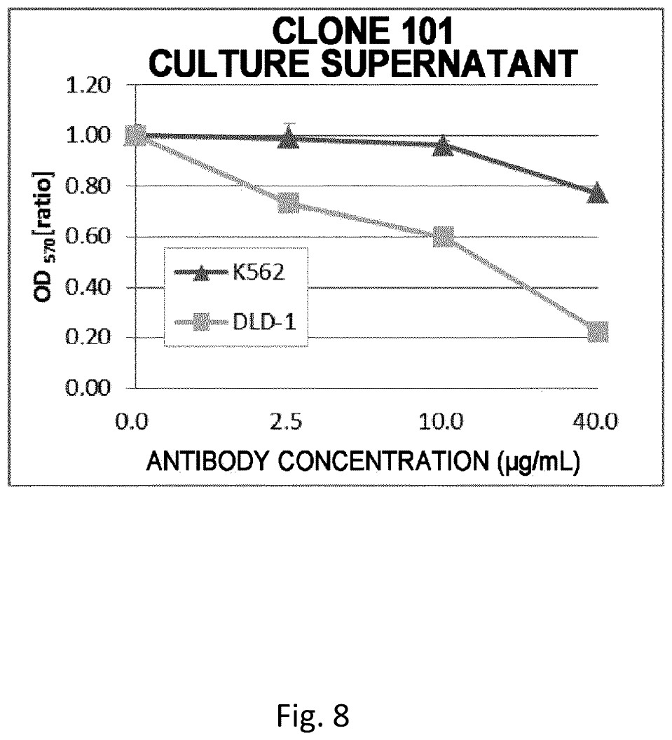

DLD-1 cells and K562 cells were added to a 96-well plate at 1×10³ cells/100 μL/well followed by culturing for 24 hours. 50 μL of medium were removed from each well of the 96-well plate followed by the addition of rat IgM antibody clone 98-producing hybridoma culture supernatant or rat IgM antibody clone 101-producing hybridoma culture supernatant, in which antibody concentration had been adjusted to 80 μg/mL, 20 μg/mL or 5 μg/mL, at 50 μL/well. Here, n number in each culture condition was 3, and wells containing medium only were prepared as controls. is added After culturing for 96 hours, WST-8 (Dojindo Laboratories, Cell Counting Kit-8) was added at 10 μL/well followed by measuring absorbance at 450 nm with a microplate reader after culturing for 3 hours. The relative values of absorbance at each antibody concentration were plotted by plotting antibody concentration on the horizontal axis and assigning a value of 1 to the optical absorbance of the well containing medium only. The results are shown in FIGS. 7 and 8. Both clone 98 and clone 101 only demonstrated cytocidal effects against colon cancer DLD-1 cells and did not demonstrate cytocidal effects against hematologic tumor K562 cells. Cytocidal effects were also observed for other colon cancer cells Difi, Car1, SW480 and Colo201. In addition, similar effects were observed for brain tumor LN229 cells and breast cancer MCF7 cells. Cytocidal effects are therefore predicted to be demonstrated against a wide range of cancers other than hematologic tumors.

8. Determination of the Base Sequences and CDR of the Variable Portion of Each Clone

1) Extraction of Total RNA

Total RNA was extracted from each antibody-producing hybridoma using the RNeasy Mini Kit (Qiagen).

2) Production of cDNA

cDNA was synthesized with the SMARTer RACE cDNA Amplification Kit (Takara Bio Inc.) according to the 5′-end rapid amplification of cDNA ends (RACE) method using the total RNA obtained in the manner described above.

3) Cloning of Anti-TMEM-180 Antibody Gene

The target gene was amplified by using PrimeStar HS DNA Polymerase (Takara Bio Inc.) on the above-mentioned cDNA. Amplification was carried out by touchdown PCR under amplification condition of 5 cycles of denaturing for 10 seconds at 98° C. and annealing/extending for 90 seconds at 72° C., 5 cycles of denaturing for 10 seconds at 98° C., annealing for 5 seconds at 67° C. and extending for 90 seconds at 72° C., and 25 cycles of denaturing for 10 seconds at 98° C., annealing for 5 seconds at 62° C. and extending for 90 seconds at 72° C.

PCR device: Takara PCR Thermal Cycler Dice Gradient

Primer sequences used:

Forward primer:

(SEQ ID NO: 26)

CTAATACGACTCACTATAGGGCAAGCAGTGGTATCAACGCAGAGT
Forward primer:

(SEQ ID NO: 27)

CTAATACGACTCACTATAGGGC
Reverse primer:

(SEQ ID NO: 28)

CCCATGGCCACCARATTCTYATCAGACAG

L chain primer:
Forward primer:

(SEQ ID NO: 29)

CTAATACGACTCACTATAGGGCAAGCAGTGGTATCAACGCAGAGT
Forward primer:

(SEQ ID NO: 30)

CTAATACGACTCACTATAGGGC
Reverse primer:

(SEQ ID NO: 31)

GTTGTTCAWGARGCACACGACTGAGGCA

The amplified PCR products of the H chain and L chain were phosphorylated using T4 Polynucleotide Kinase (Takara Bio Inc.). The phosphorylated PCR products were inserted into pUC19 (Invitrogen Corporation) cleaved at the SmaI site using Ligation High reagent (Toyobo Co., Ltd.). Following insertion, the PCR products were transformed to DH5a (Toyobo Co., Ltd.) and plasmids were extracted from single colonies using the Plasmid Mini Kit (Qiagen).

4) Gene Analysis

Genes of each of the cloned H chains and L chains were analyzed for gene base sequence using the ABI Prism 3100 Genetic Analyzer. The sequences of the H chain variable region and L chain variable region of each clone are shown in Tables 3 to 22.

Clone 98 H Chain (SEQ ID NO: 13)

TABLE 3

| | | |
|---|---|---|
| 1 | ATG GCT GTC CTG GTG CTG TTG CTC TGC CTG GTG ACA TTT CCA ACC TGT GTC CTG TCC CAG | 60 |
| | M A V L V L L L C L V T F P T C V L S Q | |
| 61 | GTG CAG CTG AAG GAG TCA GGA CCT GGT CTG GTG CAG CCC TCA CAG ACC CTG TCC CTC ACC | 120 |
| | V Q L K E S G P G L V Q P S Q T L S L T | |
| 121 | 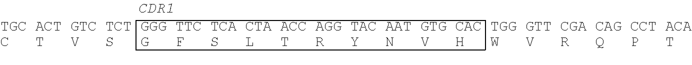 | 180 |
| 181 | 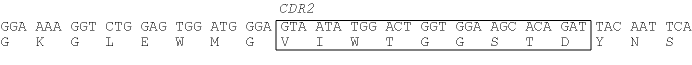 | 240 |
| 241 | GCT CTC AAA TCC CGA CTG AGC ATC AGC AGG GAC ACC TCC AAG AGC CAA GTT TTC TTA AAA | 300 |
| | A L K S R L S I S R D T S K S Q V F L K | |
| 301 | 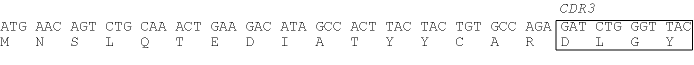 | 360 |
| 361 | TGG GGC CAA GGA GTC ATG GTC ACA GTC TCC TCA | 393 |
| | W G Q G V M V T V S S | |

Clone 98 L Chain (SEQ ID NO: 14)

TABLE 4

| | | |
|---|---|---|
| 1 | ATG ATG AGT CCT GCC CAG TTC CTG TTT CTG CTA ATG CTC TGG ATC CAG GAA ACC CAC AGT | 60 |
| | M M S P A Q F L F L L M L W I Q E T H S | |
| 61 | GAT ATT GTG ATG ACA CAG ACT CCA CTC TCT TTG TCA GTT GCC ATT GGA CAA TCA GCC TCC | 120 |
| | D T V M T Q T P L S L S V A I G Q S A S | |
| 121 | 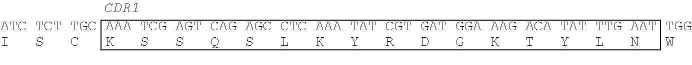 | 180 |
| 181 | 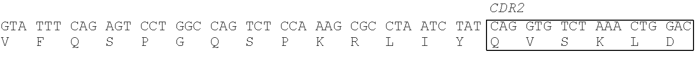 | 240 |
| 241 | 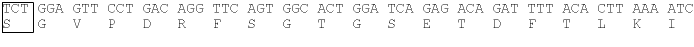 | 300 |
| 301 | 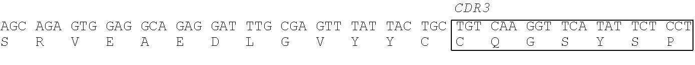 | 360 |
| 361 | 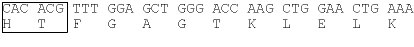 | 396 |

Clone 101 H Chain (SEQ ID NO: 15)

TABLE 5

| | | |
|---|---|---|
| 1 | ATG GCT ATC CTG GTG CTG CTT CTC TGC CTG GTG ACC ATT CCA CAC TCT GTC TTG TCC CAG | 60 |
| | M A I L V L L L C L V T I P H S V L S Q | |
| 61 | GTG CAG CTG AAG GAG ACA GGA CCT GGC CTG GTG CAA CCA ACA CAG ACC CTG TCC ATC ACA | 120 |
| | V Q L K E T G P G L V Q P T Q T L S I T | |
| 121 | 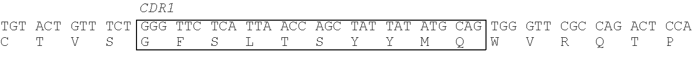 | 180 |
| 181 | 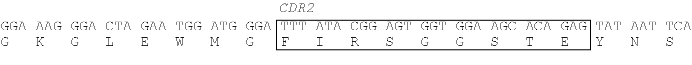 | 240 |
| 241 | GAG TTC AAA TCC CGA CTT AGC ATC AGC AGG GAC ACC TCC AAG AAC CAA GTT TTC TTA AAA | 300 |
| | E F K S R L S I S R D T S K N Q V F L K | |
| 301 | 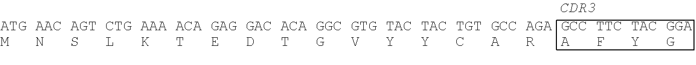 | 360 |
| 361 | 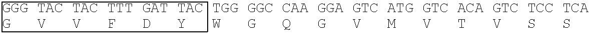 | 411 |

Clone 101 L Chain (SEQ ID NO: 16)

TABLE 6

| | | |
|---|---|---|
| 1 | ATG GGC ATC AGG ATG GAG TCA CAT ACT AGG GTC TTC ATA TTC CTG CTG CTC TGG TTG TCT | 60 |
| | M G I R M E S H T R V F I F L L L W L S | |
| 61 | GGT GCT GAT GGG GAC ATT GTG ATG ACT CAG TCT CCC ACA TCC ATT TCC ATA TCA GTA GGA | 120 |
| | G A D G D I V M T Q S P T S I S I S V G | |
| 121 | 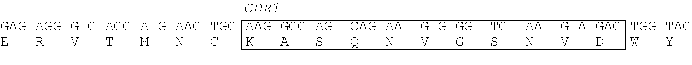 | 180 |
| 181 | 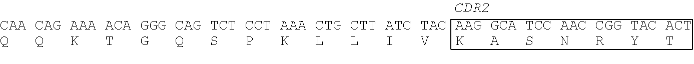 | 240 |
| 241 | GGC GTC CCT GAT CGC TTC ACA GGC AGT GGA TCT GGA ACA GAT TTC ACT TTC ACC ATC AGC | 300 |
| | G V P D R F T G S G S G T D F T F T I S | |
| 300 | 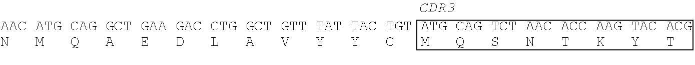 | 360 |
| 361 | TTT GGA GCT GGG ACC AAG CTG GAA CTG AAA | 390 |
| | P G A G T K L E L K | |

Clone 212 H Chain (SEQ ID NO: 46)

TABLE 7

| | | |
|---|---|---|
| 1 | ATG GAC ATC AGG CTC AGC TTG GCT TTC CTT GTC CTT TTC ATA AAA GGT GTC CAG TGT GAG | 60 |
| | M D I R L S L A F L V L F I K G V Q C E | |
| 61 | GTG CAG CTG GTG GAG TCT GGC GGA GGA TTG GTA CAG CCT GGA AAC TCC CTG AAA CTC TCC | 120 |
| | V Q L V E S G G G L V Q P G N S L K L S | |
| 121 | 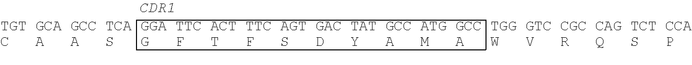 | 180 |
| 181 | 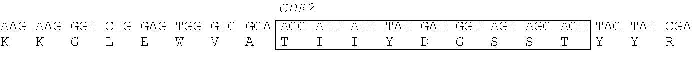 | 240 |
| 241 | GAC TCC GTG AAG GGC CGA TTC ACT ATC TCC AGA GAT AAT GCA AAA AGC ACC CTA TAC CTG | 300 |
| | D S V K G R F T I S R D N A K S T L Y L | |
| 301 | 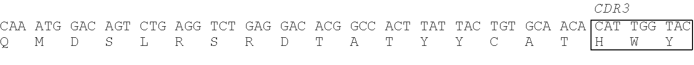 | 360 |
| 361 | 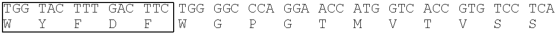 | 408 |

Clone 212 L Chain (SEQ ID NO: 47)

TABLE 8

| | | |
|---|---|---|
| 1 | ATG GGT GTG CCC ACT CAG CTC CTG GGG TTG TTG CTG CTC TGG ATT ACA GAT GCC ATA TGT | 61 |
| | M G V P T Q L L G L L L L W I T D A T C | |
| 61 | GAC ATC CAG ATG ACA CAG TCT CCA GCT TCC CTG TCT GCA TCT CTT GGA GAA ACT GTC TCC | 120 |
| | D I Q M T Q S P A S L S A S L G E T V S | |
| | CDR1 | |
| 121 | 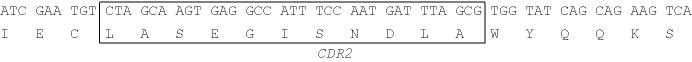 | 180 |
| 181 | 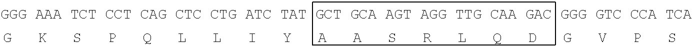 | 240 |
| 241 | CGG TTC AGT GGC AGT GGA TCT GGC ACA CGG TAT TCT CTC AAG ATC AGC GGC ATG CAA CCT | 300 |
| | R F S G S G S G T R Y S L K I S G M Q P | |
| | CDR3 | |
| 301 | 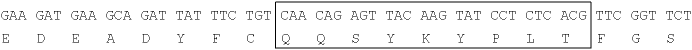 | 360 |
| 361 | GGG ACC AAG CTG GAG ATC AAA | 381 |
| | G T K L E I K | |

Clone 129 H Chain (SEQ ID NO: 54)

TABLE 9
ATG GAT TGG TTG TGG AAC TTG CTA TTC CTG ATG GTA GTT GCC CAA AGT GCT CAA GCA CAG
M D W L W N L L F L M V V A Q S A Q A Q
ATC CAG TTG GTA CAG TCT GGT CCT GAA CTG AAG AAG CCT GGA GAG TCA GTG AAG ATC TCC
I Q L V Q S G P E L K K P G E S V K I S
CDR1

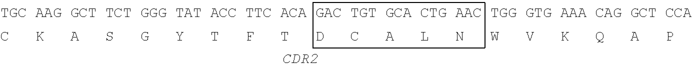

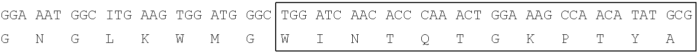

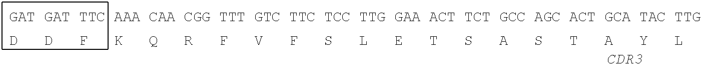

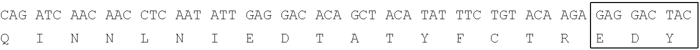

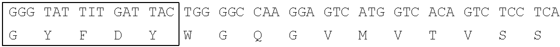

Clone 129 L Chain (SEQ ID NO: 55)

```
TABLE 10
ATG AGG ACT TCA ATT CAA CTC CTG GGG CTC TTG CTG TTG CTC TGG CTT CAT GAT GCT TGT
M R T S I Q L L G L L L L W L H D A Q C
GAC ATC CAA ATG ACA CAG TCT CCT CCC TCC CTG TCT GCA TCT CTG GGA GAC AAA GIC ACC
D I Q M T Q S P P S L S A S L G D K V T
                        CDR1

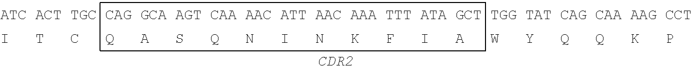

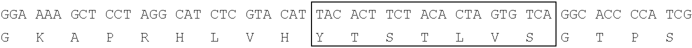


AGG TTC AGT GGC AGT GGA TCT GGG AGA GAT TAT TCA TTC AGC ATC AGC AAC GTG GAG TCT
R F S G S G S G R D Y S F S I S N V E S
                                                                CDR3

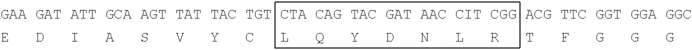

ACC AAG CTG GAA TTG AAA
T K L E L K
```

Clone 382 H Chain (SEQ ID NO: 62)

TABLE 11
ATG GAC ATC AGG CTC AGC TTG GGT TTC CTT GTC CTT TTC ATA AAA GGT GTC CAA TGT GAG
M D I R L S L G F L V L F I K G V Q C E
GTG CAG CTG GTG GAG TCT GGG GGG GGG TTG GTG CAG CCT GGA AGG TCC CTG AAA CTC TCC
V Q L V E S G G G L V Q P G R S L K L S
CDR1

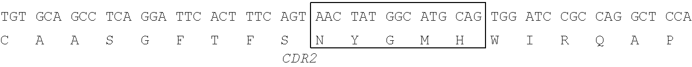

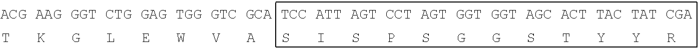

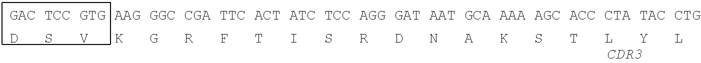

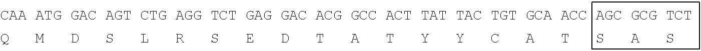

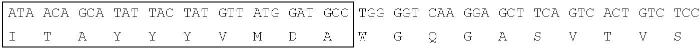

TCA
S

Clone 382 L Chain (SEQ ID NO: 63)

```
TABLE 12
ATG GAG TCA CAT ACT AGG GTC TTC ATA TTC CTG CTG CTG TGG TTG TCT GGT GCT GAT GGG
M E S H T R V F I F L L L W L S G A D G
GAC ATT GTG ATG ACT CAG TCT CCC ACA TCC ATG TCC ATA TCA GTA GGA GAC AGG GTC ACC
D I V M T Q S P T S M S I S V G D R V T
                        CDR1

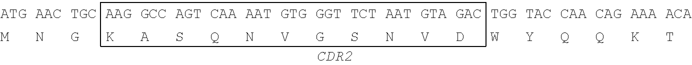

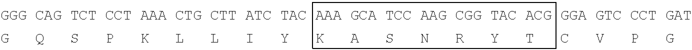


CGC TTC ACA GGC AGT GGA TCT GGA ACA GAT TTC ACT TTC ACC ATC AGG AAC ATG CAG GCT
R F T G S G S G T D F T F T I S N M Q A
                                                                CDR3

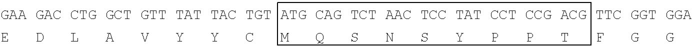

GGC ACC AAG CTG GAA TTG AGA
G T K L E L R
```

Clone 1361 H Chain (SEQ ID NO: 70)

TABLE 13
ATG GAC ATC AGG CTC AGC TTG GTT TTC CTT GTC CTT TTC ATA AAA GGT GTC CAG TGT GAG
M D I R L S L V F L V L F I K G V Q C E
GTG CAG CTG GTG GAG TCT GGG GGA GGC CTA GTG CAG CCT GGA AGG TCT CTG AAA CTA TCC
V Q L V E S G G G L V Q P G R S L K L S
CDR1

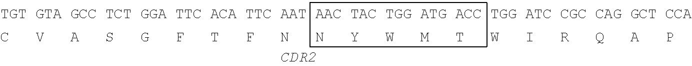

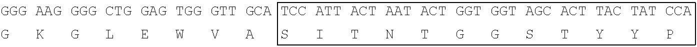

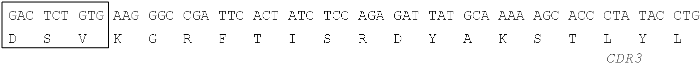

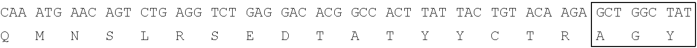

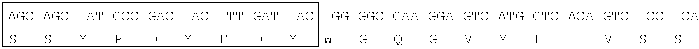

Clone 1361 L Chain (SEQ ID NO: 71)

```
TABLE 14
ATG ATG CCT CCA GTT CAA CTC TTA GGG CTG CTG CTG CTC TGG CTC CCA GCC ATG AGA TGT
M M A P V Q L L G L L L L W L P A M R C
AAC ATC CAG ATG ACC CAG TCT CCT TCA CTA CTC TCT GCA TCT GTG GGA GAC AGA GTC ACT
N I Q M T Q S P S L L S A S V G D R V T
                        CDR1

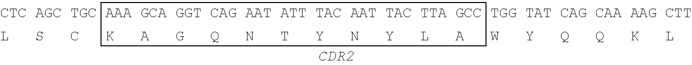

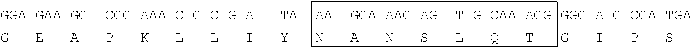


AGG TTC AGT GGC ACT GGA TCT GGT ACA GAT TTC ACA CTC ACC ATC AGC AGC CTG CAG CCT
R F S G S G S G T D F T L T I S S L O P
                                                                CDR3

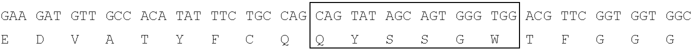

ACC AAG CTG GAA TTG AAA
T K L E L K
```

Clone 669 H Chain (SEQ ID NO: 78)

TABLE 15

| | | |
|---|---|---|
| 1 | ATG GGA TGG ATC TGT ATC ATC TTT CTT GTG GCA ACA GCT ACA GGT GTC CAC TCC CAG GTC | 60 |
| | M G W I C I I F L V A T A T G V H S Q V | |
| 61 | AAG CTG CTG CAG TCT GGG GCT GCA CTG GTG AAG CCT GGA GCC TCT GTG AAG ATG TCT TGC | 120 |
| | K L L Q S G A A L V K P G A S V K M S C | |
| | CDR1 | |
| 121 | 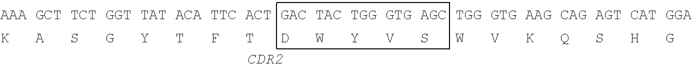 | 180 |
| 181 | 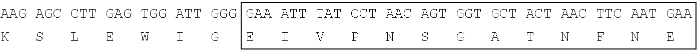 | 240 |
| 241 | 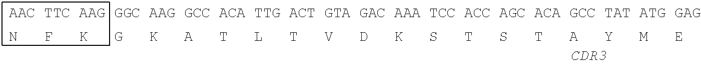 | 300 |
| 301 | 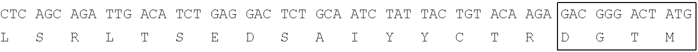 | 360 |
| 361 | 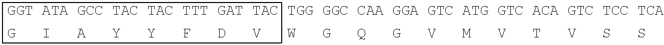 | 420 |

Clone 669 L Chain (SEQ ID NO: 79)

TABLE 16

| | | |
|---|---|---|
| 1 | ATG ATG GCT GCA GTT CAA CTC TTA GGG CTG CTG CTG CTT TGG GTC CCA GCC ATG AGA TGT | 60 |
| | M M A A V Q L L G L L L L W V P A N R C | |
| 61 | GAC ATC CAG ATG ACC CAG TCT CCT TCA TTC CTG TCT GCA TCT GTG GGA GAC AGA GTC ACT | 120 |
| | D I Q M T Q S P S V L S A S V G D R V T | |
| | CDR1 | |
| 121 | 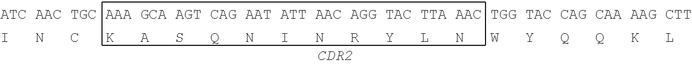 | 180 |
| 181 | 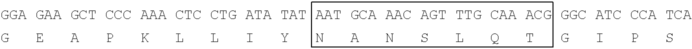 | 240 |
| 241 | CGG TTC AGT GGC AGT GGA TCT GGT ACT GAT TTC ACA CTC ACC ATC AGC AGC CTG CAG CCT | 300 |
| | R F S G S G S G T D F T L T I S S L Q P | |
| | CDR3 | |
| 301 | 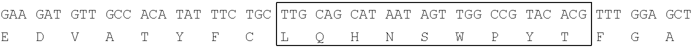 | 360 |
| 361 | GGG ACC AAG CTG GAA CTG AAA | 420 |
| | G T K L E L K | |

Clone 699 H Chain (SEQ ID NO: 86)

TABLE 17

| | | |
|---|---|---|
| 1 | ATG GAA TGG AAC TGG GTC TTT CTC TTC CTC CTG TCA GTA ACT GCA GGA GTC CAC TCC CAG | 60 |
| | M E W N W V F L F L L S V T A G V H S Q | |
| 61 | GTC CAG CTG CAG CAG TCT GGA GCT GAG CTG ACA AAG CCT GGC TCT TCA GTG AAG ATT TCC | 120 |
| | V Q L Q Q S G A E L T K P G S S V K I S | |
| | CDR1 | |
| 121 | 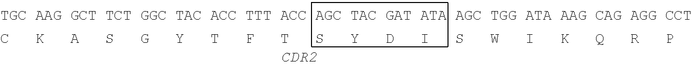 | 180 |
| 181 | 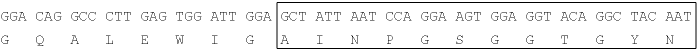 | 240 |
| 241 | 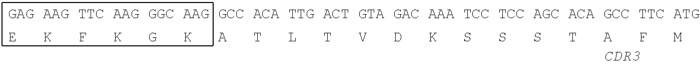 | 300 |
| 301 | 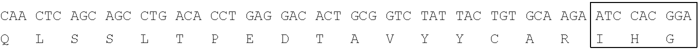 | 360 |
| 361 | 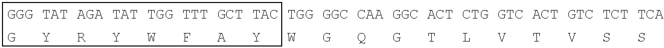 | 420 |

Clone 699 L Chain (SEQ ID NO: 87)

TABLE 18

| | | |
|---|---|---|
| 1 | ATG GAT TTT CAG GTG CAC ACT TTC AGC CTC CTG CTA ATC AGT ATC ACA GTC ATA GTG TCC | 120 |
| | M D F Q V Q S F S L L L I S I T V I V S | |
| 61 | AGT GGA GAA ATT GTG CTC ACC CAG TCT CCA ACA ACC ATG GCT GCA TCT CCA GGA GAG AAG | 180 |
| | S G E I V L T Q S P T T M A A S P G E K | |
| | CDR1 | |
| 121 | 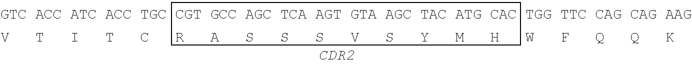 | 210 |
| 181 | 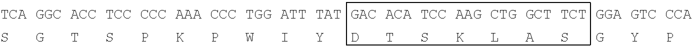 | 300 |
| 241 | GAT CGC TTC AGT GGC AGT GGG TCT GGG ACC TCT TAT TCT CTC ACA ATC AGC TCC ATG GAG | 360 |
| | D R F S G S G S G T S Y S L T I S S M E | |
| | CDR3 | |
| 301 | 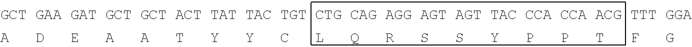 | 420 |
| 361 | GCT GGG ACC AAG CTG GAA CTG AAA | 480 |
| | A G T K L E L K | |

Clone 1052 H Chain (SEQ ID NO: 94)

TABLE 19

| | | |
|---|---|---|
| 1 | ATG GCT GTC CTG GTG CTA TTG CTC TGC CTG GTG ACA TTT CCA AGC TGT GTC CTG TCC CAG | 60 |
| | M A V L V L L L C L V T F P S C V L S Q | |
| 61 | GTG CAG CTG AAG GAG TCA GGA CCT GGC TTG ATG CAG CCC TCA GAG ACC CTG TCC CTC ACC | 120 |
| | V Q L K E S G P G L M Q P S E T L S L T | |
| | CDR1 | |
| 121 | 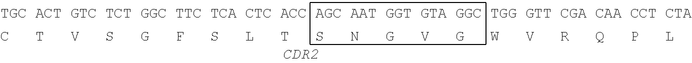 | 180 |
| 181 | 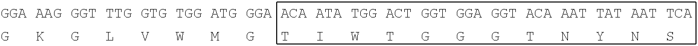 | 240 |
| 241 | 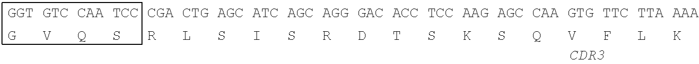 | 300 |
| 301 | 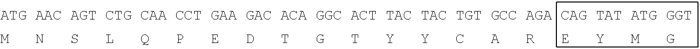 | 360 |
| 361 | 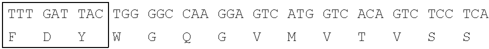 | 420 |

Clone 1052 L Chain (SEQ ID NO: 95)

TABLE 20

| | | |
|---|---|---|
| 1 | ATG GAG TTA ATC AGT CAG GTC TTC GTA TTT CTG CTG CTC TGG TTG TCT GGG GTT TAT GGG | 120 |
| | M E L I S Q V F V F L L L W L S G V Y G | |
| 61 | AAT ACT GTG ATG ACC CAG TCT CCC ACA TCT ATG TTC ACA TCA GTA GGA GAC AGG GTT ACC | 180 |
| | N T V M T Q S P I S M F T S V G D R V T | |
| | CDR1 | |
| 121 | 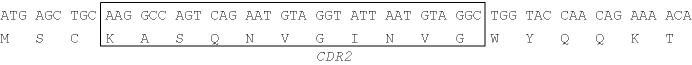 | 210 |
| 181 | 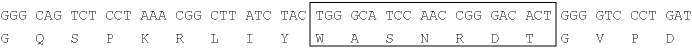 | 300 |
| 241 | GCG TTC ACA GGC AGT GGA TCT GGG ACA GAT TTC ACT CTC ACC ATC AGC AAC ATG CAG GCT | 360 |
| | R F T G S G S G T D F T L T I S N M Q A | |
| | CDR3 | |
| 301 | 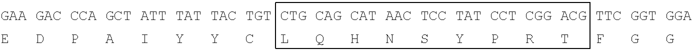 | 420 |
| 361 | GGC ACC AAG CTG GAA TTG AAA | 480 |
| | G T K L E L K | |

Clone 1105 H Chain (SEQ ID NO: 102)

TABLE 21

| | | |
|---|---|---|
| 1 | ATG GCT GTC CTG GTG CTA TTG CTC TGC CTG CTG ACA TTT CCA AGC TGT CTG CTG TCC CAG | 60 |
| | M A V L V L L L C L V T F P S C V L S Q | |
| 61 | GTG CAG CTG AAG GAG TCA GGA CCT GGC TTG ATG CAG CCC TCA GAG ACC CTG TCC CTC ACC | 120 |
| | V Q L K E S G P G L M Q P S F T L S L T | |
| | CDR1 | |
| 121 | 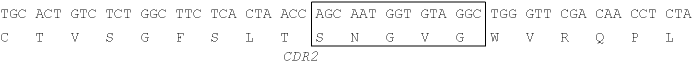 | 180 |
| 181 | 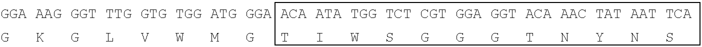 | 240 |
| 241 | 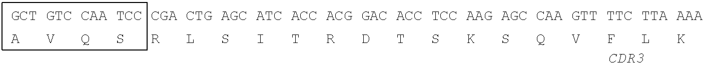 | 300 |
| 301 | 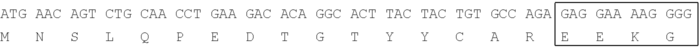 | 360 |
| 361 | 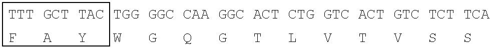 | 402 |

Clone 1105 L Chain (SEQ ID NO: 103)

TABLE 22

| | | |
|---|---|---|
| 1 | ATG GAG TTA ATC AGT CAG GTC TTC GTA TTT CTG CTG CTC TGG TTG TCT GGG GTT TAT GGG | 60 |
| | M E L I S Q V F V F L L L W L S G V Y G | |
| 61 | AAC ATT GTG ATG ACC CAG TCT CCC ACA TCT ATG TCC ACA TCA GTA GGA GAC AGG GTT ACC | 120 |
| | N T V M T Q S P T S M S T S V G D R V T | |
| | CDR1 | |
| 121 | 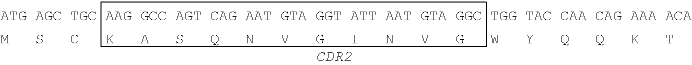 | 180 |
| 181 | 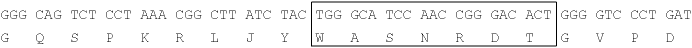 | 240 |
| 241 | CGC TTC ACA GGC AGT GGA TCT GGG ACA GAT TTC ACT CTC ACC ATC AGC AAC ATG CAG GCT | 300 |
| | R F T G S G S G T D F T L T I S N M Q A | |
| | CDR3 | |
| 301 | 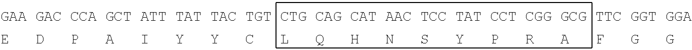 | 360 |
| 361 | GGC ACC AAG CTG GAA TTG AGA | 381 |
| | G T K L E L R | |

9. Cancer Testing Method Involving Measurement of Amount of TMEM-180

1) ELISA Protocol

First, the following indicates the protocol for the ELISA method used in the following examples.

(Reagents)

The following reagents were used.
96-well plate: C8 Maxi Breakapart (Nunc Co., Ltd. #473768)
Plate washing solution: PBS/0.05% Tween 20
Blocking solution: PBS/1% BSA
Antibody diluent: PBS/0.05% Tween 20/1% BSA
Primary antibody: IgM 98 (hybridoma culture supernatant)
Secondary antibody: Polyclonal rabbit anti-rat immunoglobulins/HRP (Dako #P0450)
Color developing solution: 1-Step Slow TMB-ELISA Substrate (Thermo Fisher Scientific Inc. #34024)
Stopping solution: 2N H₂SO₄

(Procedure)

Testing was carried out according to the procedure indicated below.

(i) Antigen Immobilization

Antibody culture supernatant or human serum (diluted 1/10 and 1/50) was added to a 96-well plate at 50 μL/well followed by incubating overnight at 4° C.

(ii) Blocking

The antigen-immobilized plate was repeatedly washed five times with plate washing solution at 200 μL/well. Blocking was then carried out with blocking solution at 200 μL/well followed by incubating for 1 hour at room temperature.

(iii) Primary Antibody Reaction

The antigen-immobilized plate was repeatedly washed five times with plate washing solution at 200 μL/well. IgM 98 antibody solution was then added at 100 μL/well and 10 μg/mL followed by incubating for 1 hour at room temperature.

(iv) Secondary Antibody Reaction

The antigen-immobilized plate was repeatedly washed five times with plate washing solution at 200 μL/well. Secondary antibody diluted 4000-fold was added at 100 μL/well followed by incubating for 1 hour at room temperature.

(v) Color Development and Stopping of the Reaction

The antigen-immobilized plate was repeated washed five times with plate washing solution at 200 μL/well. Color developing solution was then added at 100 μL/well followed by incubating for 15 minutes at room temperature. Stopping solution was then added at 100 μL/well.

(vi) Measurement

Absorbance at 450 nm was measured after stopping the reaction.

2) Measurement of Amount of TMEM-180 Protein in Cancer Cell Culture Supernatant

### (Preparation of TMEM-180-Forcibly Expressed Line)

Plasmid RC219636 (Origen) comprising human TMEM180 cDNA (NM_024789.2) were transfected into DLD-1 cells with Lipofectamine LTX (Invitrogen). Human TMEM-180 expressing lines were subjected to drug selection using Geneticin (Invitrogen) to obtain a resistant line. The obtained resistant line was subjected to limited dilution to confirm the reactivity of the antibody with FACS for each single clone. The obtained line was further subjected to maintenance culture in DMEM (Wako), 10% FBS, 1% penicillin, streptomycin, amphoterin B (Wako), 600 μg/mL of Geneticin (Invitrogen).

### (Preparation of TMEM-180 Knockdown Line)

The TMEM-180 knockdown line was generated by using MISSION™ shRNA Lentiviral Transduction Particles (Sigma-Aldrich Corporation, St. Louis, MO) according to the manufacturer's manual for each of the colon cancer cell lines DLD-1, HCT116, HCT15 and Colo320 purchased from American Type Culture Collection (ATCC, Manassas, VA). Knockdown of TMEM-180 gene expression was confirmed by a quantitative PCR using TaqMan® Gene Expression Assays (Life Technologies Corporation, Carlsbad, CA).

(Measurement of the Amount of TMEM-180 Protein in the Culture Supernatant from Cancer Cells)

Colon cancer cell line DLD-1 that forcibly expresses TMEM-180, the parent line and the TMEM-180 knockdown line were cultured and washed with PBS after nearly reaching confluency. The medium was replaced with serum-free DMEM medium followed by culturing overnight and recovering the supernatant on the following day. This supernatant was used as sample antigen and immobilized on a 96-well plate followed by carrying out ELISA according to the method described in section 1). In addition, a similar experiment was conducted on brain tumor cell line LN229.

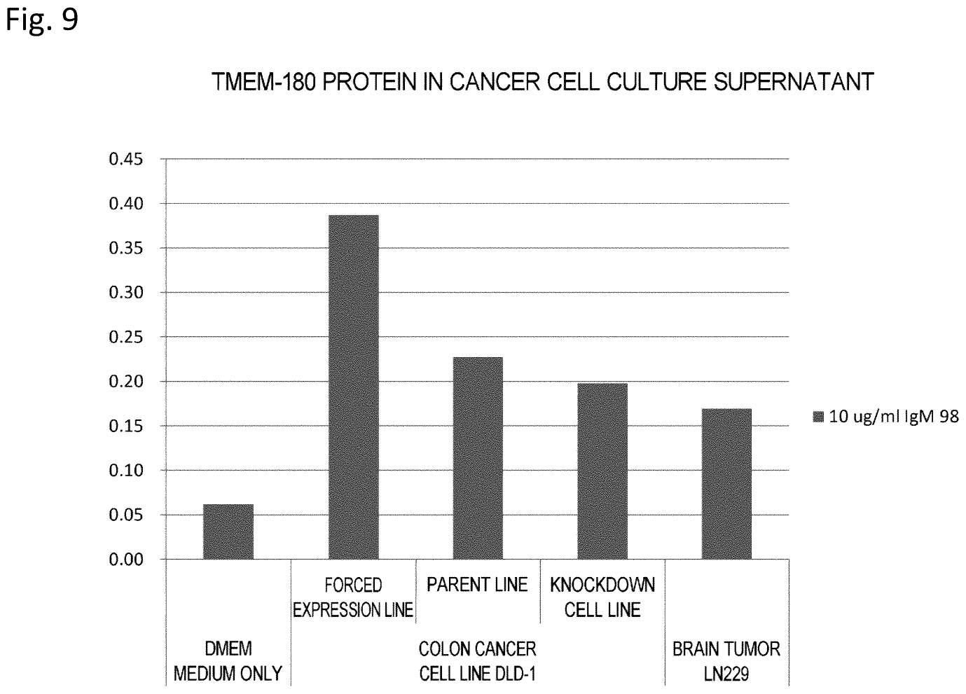

The results are shown in FIG. 9. In comparison with the normal control (medium only), TMEM-180 exhibited high levels in all of the samples. The forced expression cell line, parent cell line and knockdown cell line demonstrated high levels in that order in colon cancer DLD-1. High levels were also demonstrated in brain tumor cells.

3) Measurement of Amount of TMEM-180 Protein in Plasma of Stage IV Colon Cancer Patients

After diluting human plasma collected in EDTA (formed of four samples from stage IV patients and normal subjects) at dilution factors of 1/10 and 1/50, the plasma was used as sample antigen and immobilized on a 96-well plate followed by carrying out ELISA according to the method described in section 1).

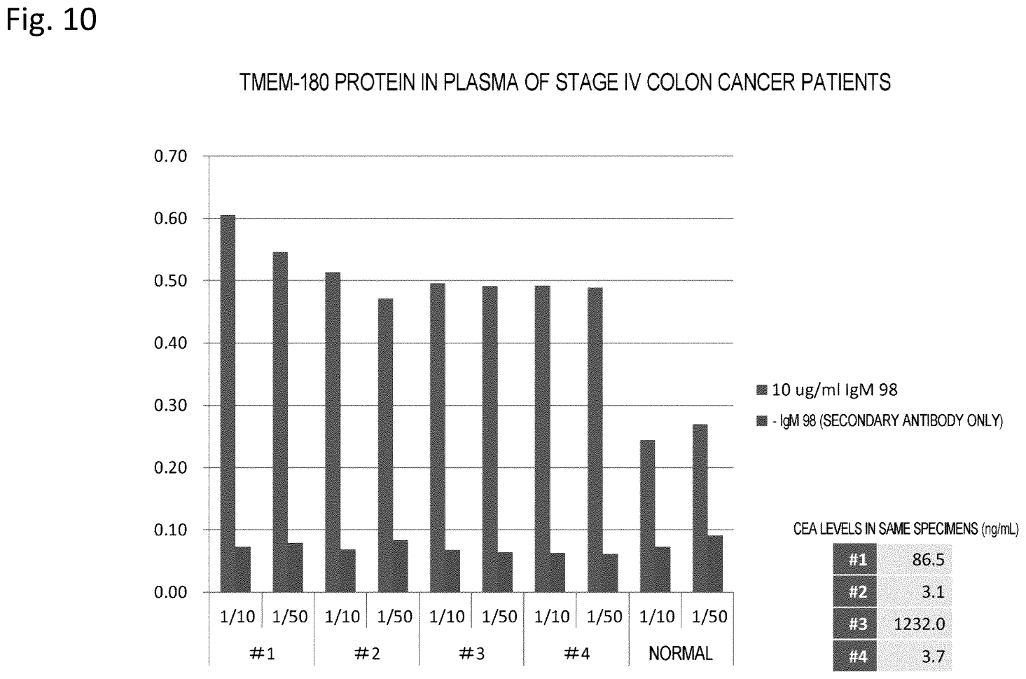

The results are shown in FIG. 10. In comparison with normal plasma, high levels were demonstrated in all of the patient samples. In addition, plasma TMEM-180 levels were positive (higher than normal) even in patients #2 and #4 who were negative for CEA.

4) Measurement of Amount of TMEM-180 Protein in Plasma of Stage III Colon Cancer Patients Before and After Surgery

The amount of TMEM-180 protein in the plasma of stage III colon cancer patients before and after surgery was measured using the same method as section 3).

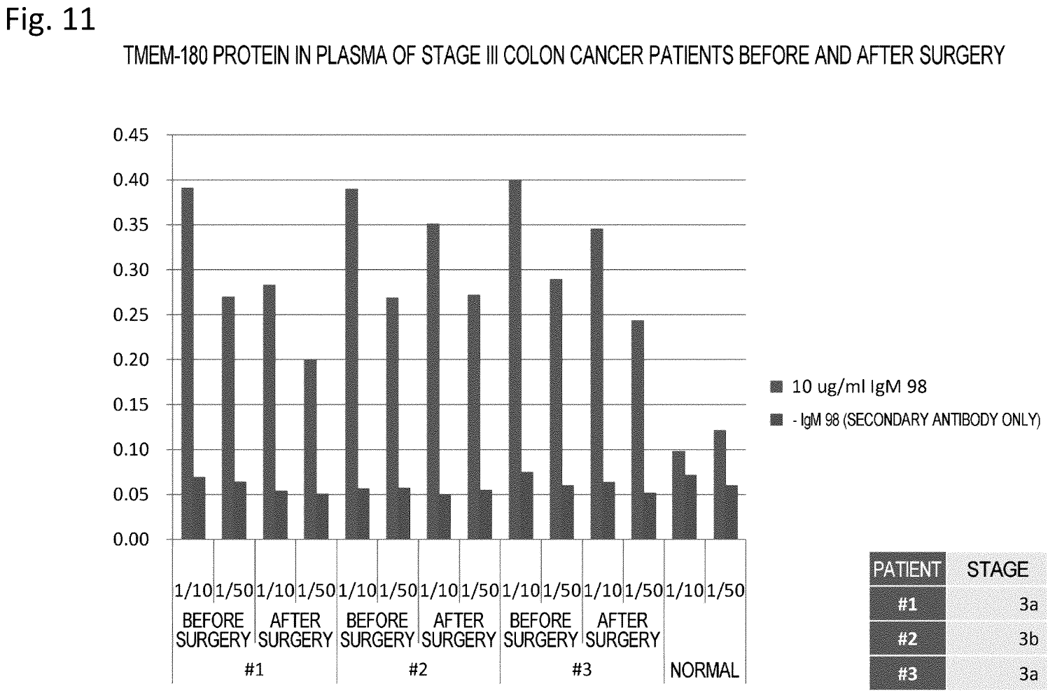

The results are shown in FIG. 11. TMEM-180 levels decreased after surgery in comparison with before surgery in all of the patients.

5) The amount of TMEM-180 protein and the colon cancer tumor marker, CEA, were measured in the plasma of stage III, II, IV and IIIa colon cancer patients before and after surgery and at the time of recurrence. Measurement of TMEM-180 was carried out by sandwich ELISA using clone 669 and clone 1361. The average of the levels of TMEM-180 in the normal plasma of 8 subjects as measured by ELISA in the same manner as in the case of the patient samples was used for the TMEM-180 cutoff value. The normal value used at the National Cancer Center Japan was used for the CEA cutoff value.

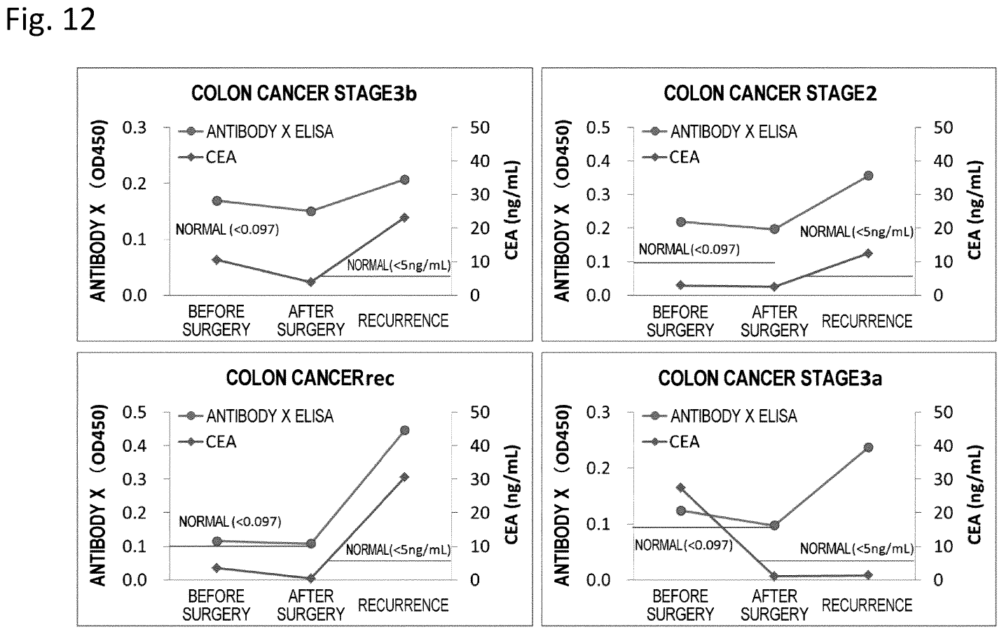

The results are shown in FIG. 12. Stage II patients were positive for TMEM-180 despite being negative for CEA level prior to surgery. In addition, although stage IIIa patients remained negative for CEA even after confirmation of recurrence by CT, TMEM-180 levels increased again.

### Example 10A. Comparison in Cancer-Detection Frequency Using Each Clone

Cancer-detection rates were tested using monoclonal antibodies (the 669 antibody, 1052 antibody, 1105 antibody, and 1361 antibody).

In this Example, the reactivity of the antibodies to the cell lines derived from cancers which were obtained from ATCC and JCRB was analyzed by FACS.

The results are shown in Table 23 below.

TABLE 23
Detection Sensitivity of Cancers with antibodies

| | Antibody Clone | | | | | | | |
|---|---|---|---|---|---|---|---|---|
| Cancer | 1361 | | 669 | | 1052 | | 1105 | |
| Type | Percentage (%) | P/N* | Percentage (%) | P/N* | Percentag (%) | P/N* | Percentage (%) | P/N* |
| Colon | 70 | (7/10) | 30 | (3/10) | 30 | (3/10) | 30 | (3/10) |
| Gastric | 63 | (5/8) | ND | | 25 | (2/8) | 25 | (2/8) |
| Pancreas | 100 | (9/9) | 33 | (3/9) | 44 | (4/9) | 44 | (4/9) |
| Brest | 33 | (2/6) | ND | | 50 | (3/6) | 50 | (3/6) |
| Prostate | 67 | (2/3) | ND | | ND | | ND | |
| Kidney | 100 | (3/3) | ND | | ND | | ND | |
| Bladder | 75 | (3/4) | ND | | 50 | (2/4) | 50 | (2¥4) |
| Brain | 100 | (7/7) | ND | | ND | | ND | |

*“P” indicates the number of positive clones and “N” indicates the number of negative clones.
*ND indicates that no data was obtained in the Example.

As shown in Table 23, it was found that any one of the antibodies produced by the clones was able to definitely recognize the cancers in the tissue specimens. It was also found that the 1361 antibody can detect several cancers with the highest sensitivity.

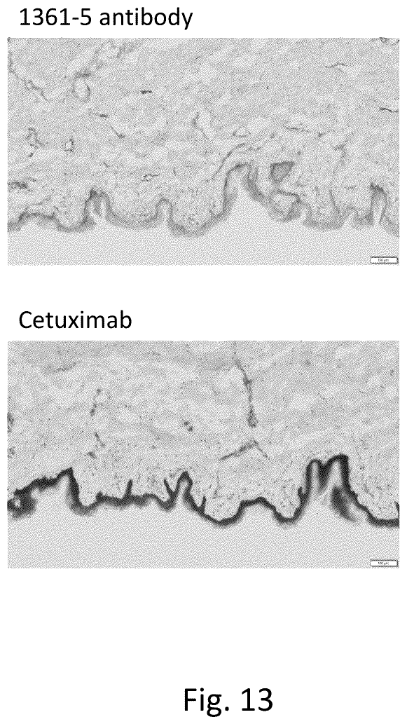

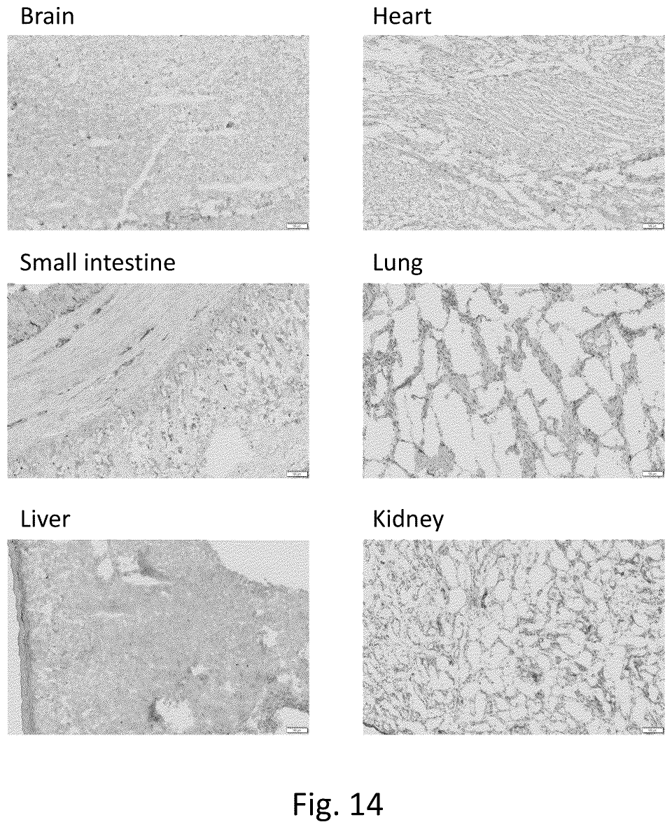

Next, the cancer-detection frequency by the antibodies was examined using various human tissue specimens which were obtained from those who had been diagnosed to have cancer. The paraffin sections were prepared from the tissue specimens in the same way as shown in Example 6 according to a routine procedure. The prepared sections were subjected to reactions with 1 μg/mL of one of the above antibodies. An HRP-labelled anti rat antibody was used as a secondary antibody, which emitted light with DAB. The observation was performed after hematoxylin staining. 1361 antibody-staining was negative in immune histochemistry of normal tissues from skin, brain, small intestine, liver, heart, lung and kidney. On the contrary, cetuximab, which is an EGFR blocker, showed a strong positive reaction to normal skin tissues. FIG. 13 shows the difference of the reactivities to skin tissue between the 1361 antibody and cetuximab. FIG. 14 shows the results of the immune histochemistry of other tissues besides skin.

Cetuximab exhibits strong reactivity to normal skin, which was consistent with the fact that skin disorders were reported as adverse effects. On the contrary, the 1361 antibody is expected to reduce such adverse effects due to the low reactivity to skin tissues.

### Example A11. Preparation and Evaluation of the Variants of 1361 Clone

Site specific mutations were introduced in the heavy chain and the light chain by an inverse PCR method to obtain variants of the 1361 clone.

(1) Preparation of Variants of 1361 Clone

One amino acid substitution Y78A, where tyrosine located in heavy chain CDR2 in the 1361 clone was replaced with alanine, was introduced using the forward primer (SEQ ID NO: 183), the reverse primer (SEQ ID NO: 184), and PrimeSTAR Max DNA polymerase (takara, R045A) by an inverse PCR method (e.g., Ochman, H. et al., Genetics, 120(3): 621-623, 1988). Additional twenty-two variants of the 1361 clone into which an amino acid mutation was introduced were prepared in a similar way. For example, the variant 1361-7 was a variant having an amino acid substitution S82A where serine at position 82 was replaced with alanine in the 1361 antibody, using the forward primer (SEQ ID NO: 185) and the reverse primer (SEQ ID NO: 186) as PCR primers for the modification. After the introduction, the antibody gene sequences and the introduction of the mutations were confirmed with ABI PRISM 3100 Genetic Analyzer. Human chimera antibodies were prepared from the obtained variants according to the method described in 3) of Example 5 above. The Human chimera clone into which one amino acid substitution Y78A substituting tyrosine located at the heavy chain CDR2 with alanine was introduced is referred to as “1361-5 clone”, and the antibody produced from the clone is referred to as “1361-5 antibody”. The 1361-5 antibody is an IgG1 antibody.

(2) Evaluation of the Binding Properties of the Antibodies by Flowcytometry

TMEM-180-forcibly expressed line of the colon cancer cell DLD-1 line, the parent line thereof, and the TMEM-180 knockdown line were cultured. Then, the binding properties of the obtained 1361-5 antibody to these cancer cell lines were analyzed below. K562 cells were used as a negative control.

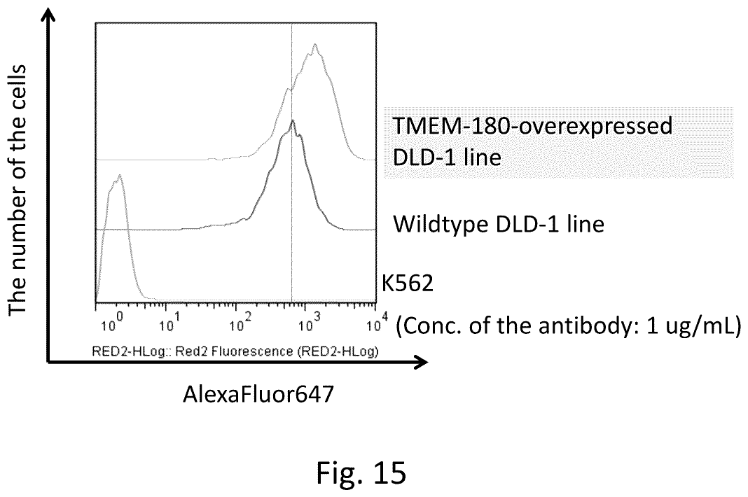

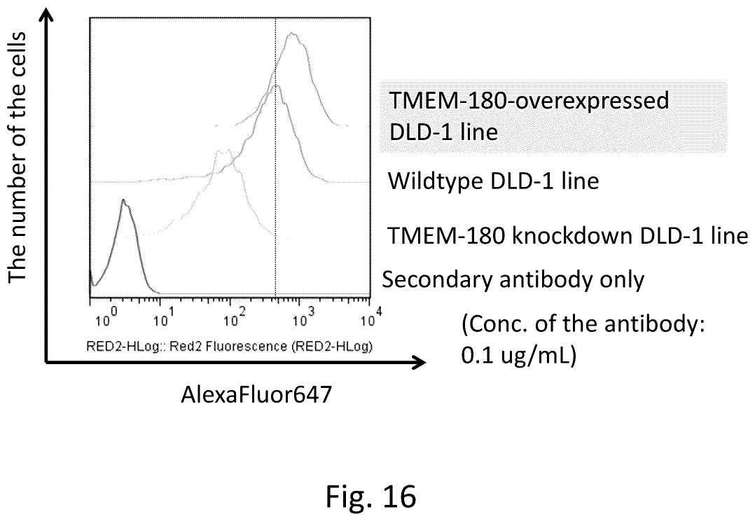

The cancer cell lines to be subjected to the measurement were suspended in the medium, and then were added to a V-bottom, 96 well plate (corning) at 1×10⁵ cells/well. After the plate was centrifuged at 440×g for 3 minutes at 4° C., the supernatant was removed and the antibody solution was added at 50 μL/well to the cell pellet, which was then suspended. The final concentration of the primary antibody was 1 μg/mL or 0.1 μg/mL. After reaction for 45 minutes on ice, the pellet was washed three times with 200 μg/well of 0.1% BSA/2 mM EDTA/PBS. After the supernatant was discarded, the secondary antibody was added at 50 μL/well to the cell pellet, which was then suspended. The AlexaFluor647 Goat anti-Human IgG (H-L) (Life Technologies) diluted with 0.1% BSA/2 mM EDTA/PBS 400 times was used as a secondary antibody. After reaction for 45 minutes on ice, the cell pellet was washed three times with 0.1% BSA/2 mM EDTA/PBS 200 μL/well. After the supernatant was discarded, 50 ng/mL Propidium Iodide/0.1% BSA/2 mM EDTA/PBS was added at 200 μg/well to the cell pellet to be suspended. The cells thus stained were measured with a flow cytometer such as Guava easyCyte 8HT (Merck Millipore). The obtained data were analyzed with FlowJO (TOMY Digital Biology). The results are shown in FIGS. 15 and 16.

As shown in FIGS. 15 and 16, the 1361-5 antibody recognized DLD-1 cell line in a TMEM-180 dependent manner, while the 1361-5 antibody did not recognize K562 cells.

As for the other twenty-two clones prepared, four clones among fourteen clones where a mutation was introduced in the heavy chain CDRs showed reduced binding properties to the antigen, and three clones among eight clones where a mutation was introduced in the light chain CDRs showed reduced binding properties to the antigen, and the other clones showed comparable binding properties to TMEM-180 to that of the 1361-5 antibody. Further, in the variants which maintain their binding properties to TMEM-180 (e.g., 1361-7 clone), the reactivity to normal stroma is almost the same as that of the 1361 antibody. On the contrary, the 1361-5 antibody showed reduced reactivity to normal stroma compared with the 1361 antibody or the other variants, while no difference was observed between the 1361-5 antibody and 1361 antibody for a binding property to TMEM-180 and recognition of cancer. It was shown that the 1361-5 antibody is more preferable in view of a binding property and specificity to cancers.

### Example B11-2. Study on ADCC Activity of the Antibody

It was considered that the 1361-5 antibody would have a high ADCC activity due to its IgG subclass. In this Example, ADCC activity of the 1361-5 antibody was studied. In this Example, Fc variants were prepared and the ADCC activity of the variants was studied. The detail will be described below.

Site specific mutations were introduced in the Fc region of the heavy chain by an inverse PCR method to obtain Fc variants of the 1361-5 clone.

1) Preparation of FcDLE, a Fc-Modified Clone of 1361-5

Specifically, the triple amino acid substitutions S239D/A330L/1332E located at the Fc region of 1361-5 clone were introduced according to an inverse PCR method (e.g., Ochman, H. et al., Genetics, 120(3): 621-623, 1988) using the forward primers (SEQ ID NOS: A1, B1) and the reverse primers (SEQ ID NOS: A2, B2) and PrimeSTAR Max DNA polymerase (takara, R045A).

The obtained variant is hereinafter referred to as Variant 1.

2) Preparation of FcDEA, a Fc-Modified Clone of 1361-5

Specifically, the triple amino acid substitutions S239D/I332E/E333A located at the Fc region of 1361-5 clone was introduced according to an inverse PCR method (e.g., Ochman, H. et al., Genetics, 120(3): 621-623, 1988) using the forward primers (SEQ ID NOS: 187, 189 or 191) and the reverse primers (SEQ ID NOS: 188, 190, or 192) and PrimeSTAR Max DNA polymerase (takara, R045A).

The obtained variant is hereinafter referred to as Variant 2.

(Fc_S239D_Fw)

SEQ ID NO: 187

GGACCGGACGTCTTCCTCTTCCCCCCA
(Fc_S239D_Rv)

SEQ ID NO: 188

GAAGACGTCCGGTCCCCCCAGGAGTTC
(Fc_A330L_I332E_Fw)

SEQ ID NO: 189

CCACTGCCCGAGGAGAAAACCATCTCC
(Fc_A330L_I332E_Rv)

SEQ ID NO: 190

CTCCTCGGGCAGTGGGAGGGCTTTGTT
(Fc_I332E_E333A_Fw)

SEQ ID NO: 191

CCCGAGGCCAAAACCATCTCCAAAGCC
(Fc_I332E_E333A_Rv)

SEQ ID NO: 192

GGTTTTGGCCTCGGGGGCTGGGAGGGC

3) Study on ADCC Activity

The ADCC activities of 1361-5, Variants 1 and 2 were studied by using cetuximab as a positive control.

Specifically, TK176V (a natural killer cell line stably expressing FcγRIII) was used as an effector cell, which was obtained from Chemicals Evaluation and Research Institute, Japan. The wild type DLD-1 cell line was used as a target cell.

T176V was cultured in RPMI1640 containing 10 ng/mL IL-2 and 10% FBS. For passages, the medium was diluted with a fresh medium every 2 to 3 days. In the passage, the cells were detached by a trypsin treatment and then suspended in a fresh medium to be plated on a dish every 2 to 3 days.

The measurement of the ADCC activity was performed as described below.

First, the amount of 51Cr release was measured when only the target cells were cultured in medium as a negative control, which is referred to as “Spontaneous release measurement”. The amount of 51Cr release was measured when the all target cells were damaged by adding 0.1% Triton X-100, which is referred to as “Max release measurement.”
1) Adding DLD-1 WT labelled with 50 μCi of ⁵¹Cr to a U-bottom, 96 well plate at 5,000 cells/50 μL/well.
2) Adding TK176V at 200,000 cells/50 μL/well (ET ratio: 40):
A medium was added at 50 μL/well to the well for Spontaneous release measurement and the well for Max release measurement.
3) Adding seven dilution series of an antibody at 100 μL/well, wherein the antibody was diluted with a common ratio of 5 and the maximum concentration of 2,000 ng/mL (i.e., the final concentrations of the antibody during the reaction: 0, 0.064, 0.32, 1.6, 8, 40, 200, and 1,000 ng/mL).
4) Adding 100 μL/well of 0.2% Triton-X 100 to the well for Max release measurement, while adding 100 μL/well of the culture medium to the well for Spontaneous release measurement.
5) Centrifuging the plate at 250×g for 4 minutes followed by culturing it at 37° C. for 4 hours.
6) Centrifuging the plate at 250×g for 4 minutes.
7) Transferring 50 μL/well of the supernatant to a test tube.
8) Measuring the radiation unit (CPM) in each test tube with a gamma counter.
9) Calculating the cytotoxicity and ADCC activity using the following formula:

Cytotoxicity (%)=(CPM for each test tube−the average of Spontaneous release CPMs)/(the average of Max release CPMs−Spontaneouse release CPMs) Formula 1:

ADCC activity (%)=Cytotoxicity when the antibody is added−Cytotoxicity when no antibody is added Formula 2:
10) Calculating 50% Effective Concentration (EC50) with an analytical software Prism.

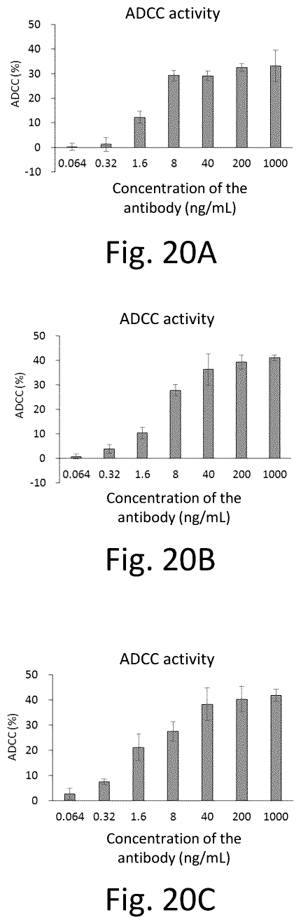

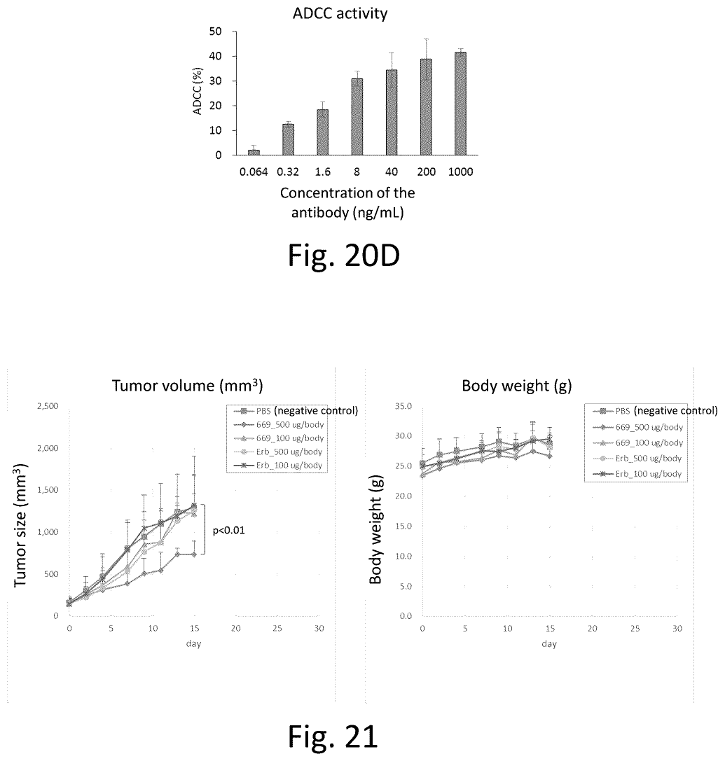

The results are shown in FIGS. 20A to 20D. As shown in FIG. 20A, cetuximab showed the strong ADCC activity against wild type DLD-1 cells. On the contrary, as shown in FIG. 20B, the 1361-5 antibody showed an ADCC activity against wild type DLD-1 cells stronger than that of cetuximab. As shown in FIGS. 20C and 20D, Variants 1 and 2 showed an ADCC activity which is equal to or stronger than that of the 1361-5 antibody. Further, no ADCC activity was observed against TMEM-180 negative cells.

From these results, it was confirmed that the 1361-5 antibody and the variants therefrom have an ADCC activity.

### Example B11-3. Effect of Anti-TMEM-180 Antibody in In Vivo Tumor Models

In this Example, the anti-tumor effect of humanized 669 IgG antibody was tested by using mouse xenograft models having human colon cancer cell lines DLD-1.

The humanized 669 IgG antibody was prepared by substituting CDRs of human IgG1 with those of the 669 antibody.

DLD-1 cells were subcutaneously injected at 5×10⁶ cells/100 μL PBS to the left abdominal sites of ICR nude mice (female, 5 to 6 weeks old, Charles River Laboratories Japan, INC.). Mice having a tumor volume of 100 to 200 mm 3 were grouped into five groups, and 500 μL of PBS, 500 μL of PBS containing 500 μg of humanized 669 IgG antibody, 500 μL of PBS containing 100 μg of humanized 669 IgG antibody, 500 μL of PBS containing 500 μg of anti-EGFR antibody Erbitux, or 500 μL of PBS containing 100 μg of Erbitux was administered to each of the groups three times per week. Each group consists of 4 to 5 mice. The tumor diameter and the weight were recorded for each of the mice upon administration. Tumor volume was calculated as [tumor major diameter×tumor minor diamiter²]. The results are shown in FIG. 21.

As shown in FIG. 21, the humanized 669 IgG antibody significantly inhibited the increase of tumor size, compared to the negative control injected with PBS. It was clear that the humanized 669 IgG antibody has an anti-tumor activity stronger than that of Erbitux. No significant change was observed in weight of mice between before and after administrations.

### Example A12. Staining of Colon Cancer Tissue Array Using the 1361-5 Antibody

In this Example, colon cancers were stained by immunohistochemistry using the 1361-5 antibody obtained in Example A11.

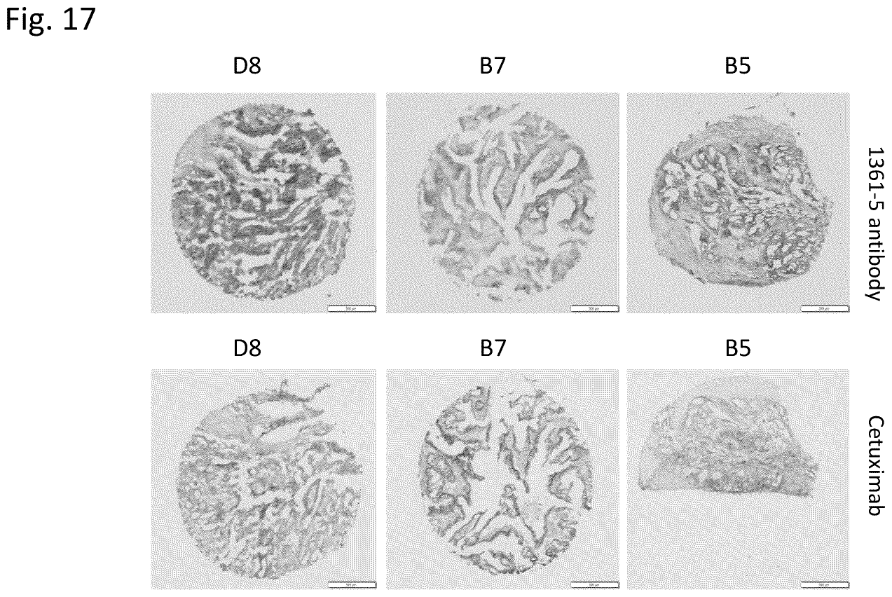

The colon cancer tissue arrays were purchased from BioChain Institute Inc. (Newark, CA) and used in the experiment. Each tissue array was stained according to the manufacturer's manual. The results are shown in FIG. 17. As shown in FIG. 17, colon cancer tissues (D8, B7 and B5) were well stained with the 1361-5 antibody (see top three panels). On the contrary, all tissues in the arrays were negative in staining with cetuximab (see bottom three panels in FIG. 17).

### Example A13. Analysis of Exosomes

In this Example, it was discovered that TMEM-180, which is a transmembrane protein, is released into exosomes from cancer cells and presented as a transmembrane protein on the surfaces of exosomes.

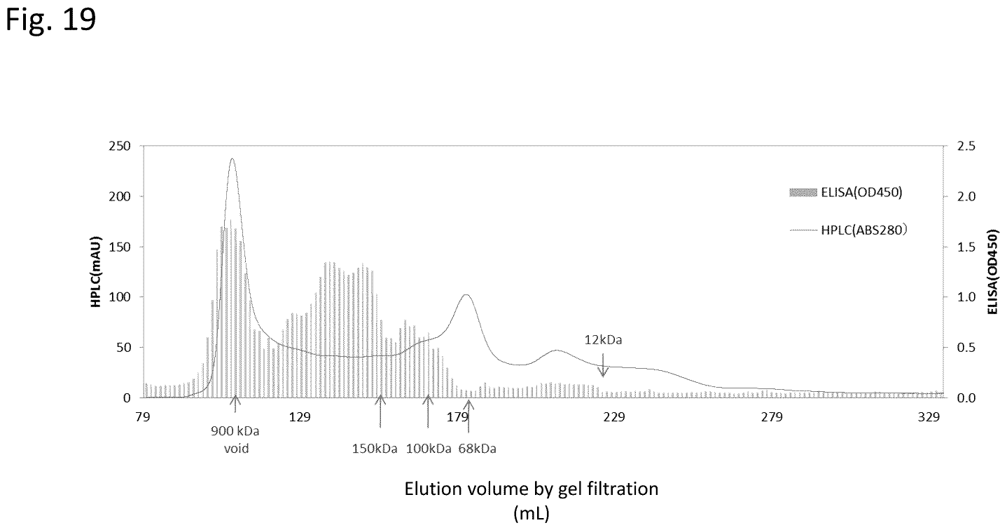

Specifically, the colon cancer cell line DLD-1 was cultured and the supernatant of the culture was obtained. After gel filtration, each fraction was checked by using the 98 antibody to find that 98 antibody positive fractions exist in large molecular weight fractions, in which the molecular weight was much larger than the molecular weight of the protein. This result suggests that TMEM-180 would form a large complex (see e.g., FIG. 19).

Thus, the inventors constructed a system for detecting exosomes in the culture supernatant. In this system, an anti-TMEM-180 antibody was immobilized on the surface of the plate, and exosomes containing TMEM-180 which were bound to the immobilized antibody were detected with an anti-CD9 antibody which CD9 is an exosome maker. The DLD-1 cell line (also referred to as “wild type DLD-1 cell line”) was used as cells. The TMEM-180 knockdown DLD-1 cell line and K562 cell line were used as negative controls.

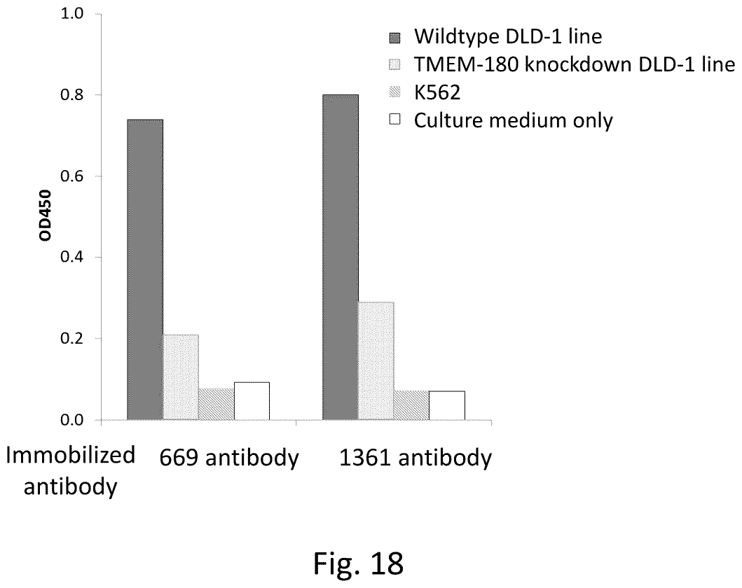

Specifically, an anti-TMEM-180 antibody was diluted with phosphate buffer such that the concentration will be 5 μg/mL, and then added to a 96 well plate (Maxisorp #442404, Thermo Fisher co., Ltd.) at 50 μL/well. The immobilization reaction was performed by incubation for 2 hours at room temperature or for overnight at 4° C. The plate after the immobilization was washed three times with 300 μL/well of a plate washing solution. The plate was blocked with 200 μL/well of a blocking agent (1% BSA/TBS-T), and allowed to stand for 1 hour at room temperature or for overnight at 4° C. After removing the blocking agent, each of the culture supernatants of wild type DLD-1 cell line, TMEM-180 knockdown DLD-1 cell line, and K562 cell line cultured in serum-free medium was added to the plate at 50 μL/well, and allowed to stand for 1 hour at room temperature. Fresh serum-free medium was used as a negative control. After the plate was washed three times with the plate washing solution, a biotin-labeled anti-CD9 antibody (156-030, Ancell co., LTD.) diluted with 1% BSA/TBS-T to have 5 μg/mL was added to the plate at 50 μL/well. After allowing the plate to stand for 1 hour at room temperature, the plate was washed three times with the plate washing solution. Streptavidin-HRP conjugated (SA-5004, Vector Laboratories) diluted with 1% BSA/TBS-T 5,000 times was added to the plate at 50 μL/well. After allowing the plate to stand for 30 minutes at room temperature, the plate was washed three times with the plate washing solution. Then, 100 μL/well of a color development solution was added to the plate and the plate was allowed to stand for 30 minutes at room temperature. The color reaction was stopped with 30 μL/well of a stop solution. Then, the optical density at 450 nm was measured with a plate reader. The results are shown in FIG. 18.

As shown in FIG. 18, the DLD-1 cell line releases exosomes to the culture supernatant, which exosomes presented TMEM-180 protein on the surfaces and were successfully detected with the 669 antibody and 1361 antibody and an anti-CD9 antibody. The amount of exosomes presenting TMEM-180 on their surfaces were reduced in the TMEM-180 knockdown cell line. The similar results were observed in the K562 cell line expressing no TMEM-180, which were comparable to the result of the medium used as a negative control.

From the above, it was discovered that exosomes presenting TMEM-180 on their surfaces are released from cancer cells and can be detected with an anti-TMEM-180 antibody and anti-exosome antibody. The inventors have found that exosomes presenting TMEM-180 on their surfaces are released in the serum obtained from human colon cancer patients. From these results, it was shown that cancers can be diagnosed by determining whether exosomes presenting TMEM-180 on their surfaces are detected or not in a body fluid sample as an index for testing cancer.

### Example B13-2. Diagnosis of Colon Cancer Using Exosomes

In this Example, it was shown that exosomes contained in the serum from colon cancer patients can be used in order to diagnose cancer.

In this Example, the components reacting with the 1361 antibody were purified from the serums from colon cancer patients (51 cases) by using 1361 antibody immobilized beads. Then, the exosomes contained in the components were detected by using an anti-CD9 antibody, in which CD9 is an exosome marker.

The detail will be provided below.

1) Preparation of Antibody-Bound Magnetic Beads

The 1361 antibody was diluted with 25 mM MES-NaOH to have 1 mg/mL. NHS-FG beads (Tamagawa seiki co., ltd., Cat. No.: TAS8848N1141) were suspended in 50 μL of 25 mM MES-NaOH per 100 μg beads and then were mixed with the equal volume of the antibody solution followed by agitation for 30 minutes at 4° C. to allow the antibody bound to the beads. After removing the supernatant, 250 μL of 1M ethanolamine (pH8.0) was added and the blocking was performed for overnight at 4° C. The beads were washed three times with 200 μL of 10 mM HEPES-NaOH (pH7.9)/50 mM KCl/1 mM EDTA/10% glycerol. The obtained antibody-bound magnetic beads were suspended in 200 μL of 10 mM HEPES-NaOH (pH7.9)/50 mM KCl/1 mM EDTA/10% glycerol in order to be used in the experiment.

2) Preparation of Positive Specimens

The DLD-1 cells were cultured overnight at 6.85×10⁶ cells per 15 cm dish. After removal of the culture supernatant, 7 mL of PBS was added to remove the culture components. After washing with PBS was performed twice, 30 mL of a serum-free medium was added and then the cells were cultured for 24 hours. The obtained supernatant of the serum-free medium was filtrated with a 0.22 μm filter and was subjected to the measurement.

3) Detection of Exosomes Containing TMEM-180

The following materials were used:
96 well plate (Corning Co., Ltd, Cat. No. 3600)
Plate washing solution 10 mM TBS (pH7.2)/0.1% Tween20/140 mM NaCl
Dilution solution 10 mM TBS (pH7.2)/0.05% Tween20/140 mM NaCl/1% BSA
Dilution solution for specimen 10 mM TBS (pH7.2)/0.1% Tween20/140 mM NaCl/1% BSA/20 μg/mL TRU Block (Meridian Life Science, Inc., Cat. No. A66800H-0.1)
Biotinylated anti-CD9 antibody (Ancell corporation, Cat. No. 156-030)
Streptavidin ALP (R&D systems, Inc., Cat. No. AR001)
Light-emitting substrate Lumipulse® substrate solution (Fujirebio Inc.)
Plate reader Spectra Max Paradigm (MolecuLar Devices, LLC.)

### (Procedures)

The experiment was performed according to the following.

### (i) Preparation of Antibody-Bound Magnetic Beads

The 1361 antibody-bound magnetic beads were suspended in Dilution solution such that the concentration will be 0.001 mg/50 μL.

### (ii) Preparation of Specimens

A positive specimen and serum specimens were diluted to 10 times.

(iii) Primary Reaction

The 1361 antibody-bound magnetic beads and a specimen were mixed in 96 well plate at 50 μL/well each, and were stirred with a plate mixer for 30 minutes to react.

### (iv) Labelled Antibody Reaction

The plate was washed three times with 300 μL/well of a plate washing solution. A biotin-labelled antibody was prepared with Dilution solution at 0.3 μg/mL. The antibody was added at 50 μL/well. The plate was stirred on a plate mixer for 30 minutes for the reaction.

### (v) Streptavidin ALP Reaction

The plate was washed three times with 300 μL/well of a plate washing solution. Streptavidin ALP solution was diluted with Dilution solution to 2,000 times. The solution was added at 50 μL/well. The plate was stirred on a plate mixer for 30 minutes for the reaction.

### (vi) Measurement

The plate was washed three times with 300 μL/well of a plate washing solution. Light-emitting substrate solution was added at 50 μL/well and then incubated for 10 minutes at room temperature. The amount of light emission was measured with a plate reader.

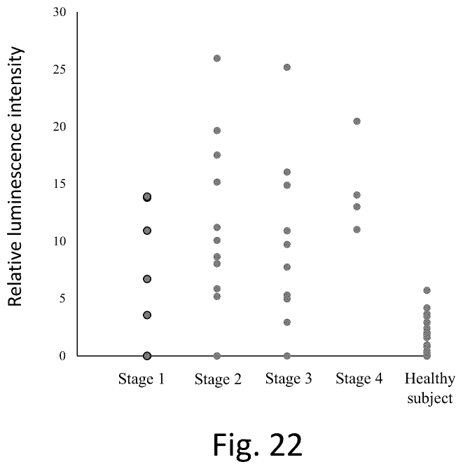

The results are shown in FIG. 22. As shown in FIG. 22, it was clear that the serums from colon cancer patients contained more TMEM-180 positive exosomes than those contained in healthy subjects.

Next, the cutoff values (thresholds) were determined by using the data shown in FIG. 22 to evaluate the sensitivity and the specificity of the diagnosis. Specifically, the average (N) of the relative luminescence intensities in the healthy subjects (50 cases) was used as a reference value, and a cutoff value (threshold) was set as a value three times (S/N=3), four times (S/N=4), or five times (S/N=5) larger than the reference value. When the relative luminescence intensity exceeds the cutoff value, the sample was determined as colon cancer and thereby verifying the specificity of this diagnosis. The results are shown in Table 24.

TABLE 24
An example of serological diagnosis using serums
from colon cancer patients (51 cases)

| | Stage 1 | Stage 2 | Stage 3 | Stage 4 | Specificity |
|---|---|---|---|---|---|
| S/N = 3 | 62.5% | 76.9% | 90.0% | 100% | 88% |
| | (5/8) | (10/13) | (9/10) | (4/4) | |
| S/N = 4 | 62.5% | 76.9% | 80.0% | 100% | 92% |
| | (5/8) | (10/13) | (8/10) | (4/4) | |
| S/N = 5 | 50.0% | 76.9% | 80.0% | 100% | 98% |
| | (4/8) | (10/13) | (8/10) | (4/4) | |

As shown in Table 24, it was found that colon cancers at every stage can be detected with a very high sensitivity according to the present invention. In particular, the stage 2 cancers were detected at the high sensitivities of over 75% by using any one of the cutoff values. Further, the stage 1 cancers were detected also at the high sensitivities of over 50%. The detection specificity was over 85% in all cases.

In the examples of above, S/N is 3 or more. However, from the results shown in FIG. 22, it is clear that cancers can be detected even when S/N is set to be 1. Generally, the sensitivity will drop while the specificity will rise, when the cutoff value is high; the sensitivity will rise while the specificity will drop, when the cutoff value is low. It can be recognized that those skilled in the art would determine the cutoff value, depending on whether the purpose is to decrease false negatives or false positive.

### Example B13-3 Observation of the Expression of TMEM-180 on Surfaces of Exosomes

In the Example, the expression of TMEM-180 on surfaces of exosomes was checked by using an electron microscope.

Harvesting Exosomes from Culture Supernatant

Each of DLD-1 cells and TMEM-180-forcibly expressed DLD-1 cells were plated on plastic dishes and were incubated overnight. After removing the culture supernatant, the cells were washed twice with PBS such that the cells were not detached from the dishes, and then the medium was exchanged with a serum-free D-MEM medium. After culturing the cells for 24 hours, the culture supernatant was harvested. The supernatant filtrated with a 0.22 μm filter was used as a serum-free culture supernatant containing exosomes. The K562 cells which were washed three times with PBS were cultured in a serum-free RPMI-1640 medium for 24 hours. The culture supernatant was harvested and filtrated with a 0.22 μm filter for use. The obtained serum-free culture supernatant containing exosomes were centrifuged at 100,000×g, 4° C. for 24 hours. The obtained pellet was suspended in PBS as an exosome concentrate.

Immunological Staining of the Exosomes

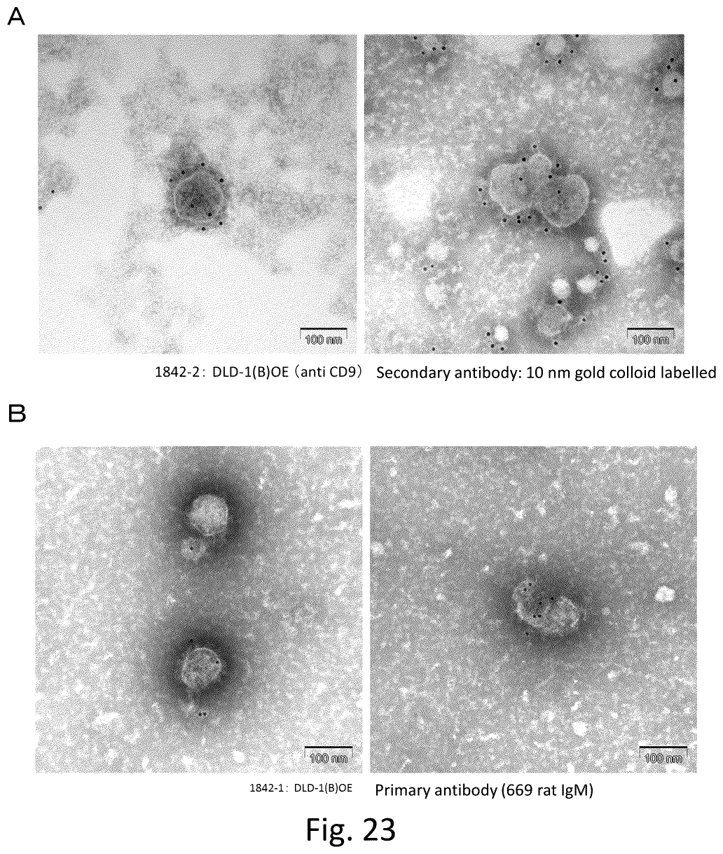

The exosome concentrate was dispersed and then attached on a Ni grid with a stretched supporting membrane. The 669 antibody, which is an anti-TMEM-180 antibody clone, or an anti-human CD9 antibody (Cosmo bio) was added to the Ni grid and reacted at room temperature. The Ni grid was washed with 1% BSA/PBS, followed by addition of an anti-mouse Ig antibody labelled with gold colloid or an anti-rat Ig antibody labelled with gold colloid. The reaction was performed at room temperature. After washing with PBS, fixation with glutaraldehyde was performed for 3 minutes. Negative staining was performed with a 2% phosphotungstic acid solution (pH7.0). The fixed Ni grid was dried, and the binding of exosomes and the antibody was checked with a transmission electron microscope. Gold colloid was detected as a black dot under transmission electron microscopy. The results are shown in FIG. 23.

As shown in FIG. 23, the exosomes associated with a plurality of black dots in the staining with an anti-CD9 antibody, which indicates that the exosomes were stained with the anti-CD9 antibody (see Panel A in FIG. 23). Further, the exosomes, which were stained with an anti-TMEM-180 antibody, also associated with a plurality of black dots (see Panel B in FIG. 23) in the staining with the anti-TMEM-180 antibody. Those from K562 cells expressing no TMEM-180 did not reacted with the anti-TMEM-180 antibody. From these results, it was shown that the exosomes released from colon cancer cells into body fluids express TMEM-180. These results support that a cancer diagnosis is possible by using exosomes and an anti-TMEM-180 antibody.

### Example A14. Preparation of an Anti-TMEM-180 Antibody by Using Exosomes as an Antigen

After it was shown that TMEM-180 protein was detected on the exosomes, an anti-TMEM-180 antibody was prepared by using the exosomes of above as an antigen instead of the antigen used in Example 3.

Preparation of Immunogen

### 1) Preparation of Serum-Free Culture Supernatant

A TMEM-180-overexpressed DLD-1 cell line was cultured in a 15 cm dish (#430599, Corning Co., Ltd.) containing 30 mL of DMEM low glucose medium (Wako) containing 10% FBS at 6.75×10⁶ cells per dish, at 37° C. under the conditions of 5% CO₂ for 24 hours. The culture medium was removed from the culture dish and the dish was gently washed with PBS. Then, 30 mL/dish of D-MEM low glucose medium was added and then the cells were cultured for 24 hours. The harvested serum-free culture supernatant was filtrated with a 0.22 μm filter flask. After the cell debris was removed, Protease Inhibitor (Wako) was added to the supernatant at the ratio of 1/1000. The supernatant was stored at 4° C.

### 2) Preparation of Exosome Fraction

A column filled with CHT ceramic hydroxyapatite type II (Bio-Rad) was equilibrated with 30 mM HEPES, 100 mM NaCl, pH6.7. The serum-free culture supernatant was loaded to the column, then washed with the same buffer, and then eluted with 500 mM KPB, pH6.7. The eluent was concentrated to a volume of less than 10 mL by using ultrafiltration, and was fractionated with a gel filtration column, Superdex 200 μg (GE). Each of the fractions was subjected to the measurement of TMEM-180 positive fractions. Then, the peak of the positive fractions detected at the void volume was collected and used as TMEM-180 containing exosomes.

### 3) Measurement of TMEM-180 Positive Fractions

The samples for the measurement were diluted with the stock solution or a phosphate buffer, and were added to a 96-well plate (Maxisorp #442404, Thermo Fisher co., Ltd.) at 50 μL/well. The immobilization reaction was conducted at room temperature for 2 hours. The immobilized plate was washed three times with 300 μL/well of the plate washing solution. The plate was blocked with 200 μL/well of 1% BSA/TBS-T and left at room temperature for 1 hour. After the removal of the blocking agent, clone 98 antibody diluted with 1% BSA/TBS-T at 5 μg/mL was added to the plate at 50 μL/well. The plate was allowed to stand at room temperature for 1 hour. Then, the plate was washed three times with the plate washing solution. Goat anti rat IgM antibody HRP conjugated (A110-100P, Bethyl laboratories, Inc.) which has been diluted 10,000 times with 1% BSA/TBS-T was added to the plate at 50 μL/well. The plate was left standing at room temperature for 30 minutes, and was washed three times with the plate washing solution. The color development solution was added at 100 μL/well, and then the plate was left standing at room temperature for 20 minutes. 30 μL/well of the stop solution was added to stop the color reaction, and then the plate was subjected to the measurement of 450 nm absorbance with the plate reader.

### 4) Preparation of Antibody

TMEM-180-containing exosomes and Freund complete adjuvant were mixed at 1:1 ratio to obtain emulsion, 100 μL of which was administered to each rat (Japan SLC, Inc., Wister, female, 6-8 weeks old) at the both sides of the root of the tail. The liac lymph node, the inguinal lymph node, the axillary lymph node, and popliteal lymph node were isolated 14 days after the immunization. The obtained lymph node cells and mouse myeloma cells p3X63 were fused by a PEG method. Ten to fourteen days after the fusion, the culture supernatant was harvested. Then, the hybridomas which produced antibodies positively reacted to DLD-1 cells and negatively reacted to K562 cells were selected by flow cytometry, and then were further screened by using the reactivity to the TMEM-180-overexpressing DLD-1 cell line, the TMEM-180 knockdown DLD-1 cell line, and the TMEM-180 knockout DLD-1 cell line. The selected antibody-producing hybridoma cells were cloned to be established by a limiting dilution method. The isotype of the antibody was determined with an isotype-specific ELISA (Bethyl).

One IgG antibody and three IgM antibodies were obtained as a result of the above method. These antibodies well recognized TMEM-180 protein on the exosomes and well stained the cancer tissues, which was similar to the results in the above Example. No wonder that the antibodies obtained by immunizations with exosomes are useful for the cancer diagnoses using the exosomes. It is thought that TMET-180 has the native conformation on the exosomes and presents the same part as that presented on the cell membrane. Further, immunohistological staining and FACS showed that the antibodies, which were obtained by immunizations with exosomes, bound to TMEM-180 on cell membranes. Thus, it is thought that a method of obtaining an antibody by immunization with exosomes is useful as a method of obtaining an anti-membrane protein antibody, in particular, an anti-TMEM-180 antibody.

### Example C15. Construction of Antibody-Drug Conjugate (ADC) and Physiological Examination Thereof

In this Example, ADC was constructed, and the effects thereof on colon cancer cell lines were examined.

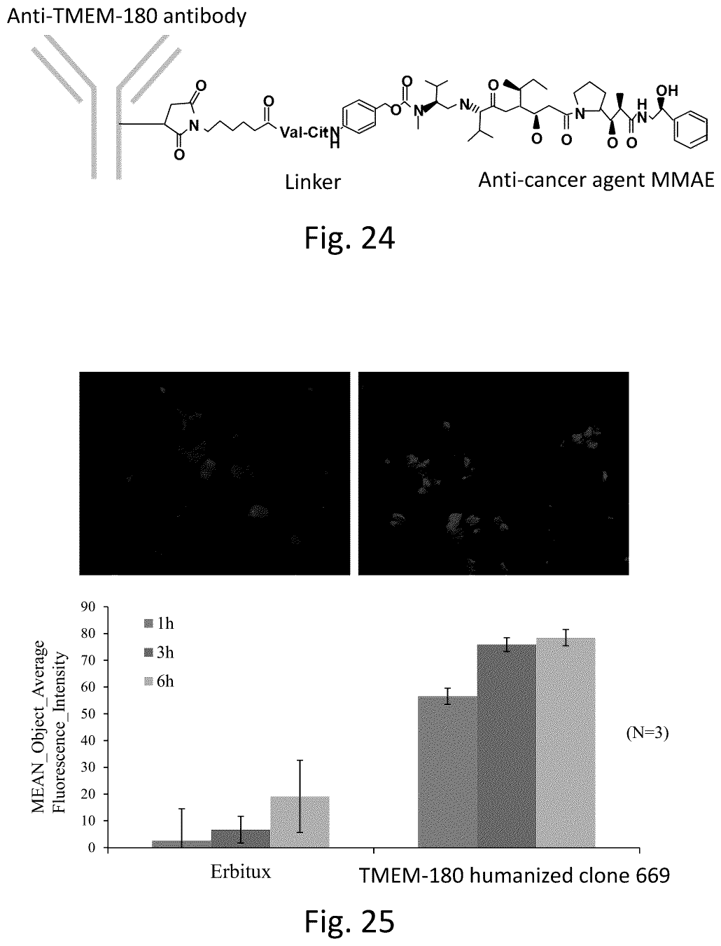

The 669 antibody or 372 antibody was used as an anti-TMEM-180 antibody and monomethyl auristatin E (MMAE) was used as a drug in ADC. The antibody and MMAE were linked via a linker. The valine-citrulline linker (Val-Cit linker), which can be cleaved with cathepsin, was used as a liker. The specific structure of the ADC is shown in FIG. 24.

### Example C15-1. Preparation of the ADC

### (1) Synthesis of the Linker

An N,N-dimethylformamide (DMF) solution (18 mL) containing Fmoc-Val-OSu (2.80 g, 6.42 mmol) was added to an aqueous solution (18 mL) containing H-Cit-OH (1.18 g, 6.74 mmol) and NaHCO (566 mg, 6.74 mmol), and then tetrahydrofuran (THF) (9 mL) was added to the solution. After overnight stirring, the reaction was stopped with a 15% citric acid solution (40 mL). The aqueous layer was extracted with the mixture solutions of AcOEt/i-PrOH (9/1) (100 mL, and 20 mL×2). A combined organic layer was washed with water (70 mL) and then was subjected to vacuum concentration. The residual solid was washed with diethyl ether and then dipeptide Fmoc-Val-Cit-OH (3.11 g, 97%) was obtained as a white solid.

To a solution containing Fmoc-Val-Cit-OH (3.00 g, 6.04 mmol) and p-aminobenzyl alcohol (PAB) (1.49 g, 12.1 mmol)-methanol (30 mL), 1-ethoxycarbonyl-2-ethoxy-1,2-dihydroquinoline (EEDQ) (2.99 g, 12.1 mmol) was added. One day later, EEDQ (1.50 g, 6.04 mmol) was further added to the solution, followed by overnight stirring. The reaction solution was concentrated and then diethyl ether was added to the residue and was washed by sonication. Thus, Fmoc-Val-Cit-PAB-OH (2.51 g, 69%) was obtained.

### (2) Connection of the Linker and MMAE and Preparation of the ADC

MMAE (1.00 g, 1.39 mmol) was added to a solution of dimethyl formamide (3.4 mL) and pyridine (0.85 mL) containing p-nitrophenyl carbonate body (1.28 g, 1.67 mmol) and 1-hydroxybenzotriazol (HOBt) (376 mg, 2.78 mmol). After 24 hours, the reaction solution was purified with Sephadex LH20 (solvents: CHCl₃:MeOH 1:1) to obtain Fmoc-Val-Cit-PABC-MMAE (1.44 g, 77%).

Et₂NH (5 mL) was added to a dimethylformamide solution (20 mL) containing Fmoc-Val-Cit-PABC-MMAE (1.44 g, 1.07 mmol). After overnight stirring, the reaction solution was subjected to vacuum concentration. The residue was washed with ethyl acetate and diethyl ether to obtain a light-yellow solid (960 mg, 80%).

Maleimide-polyethylene glycol 12-succinimidyl ester (Mal-PEG₁₂-Osu) (814 mg, 0.94 mmol) and diisopropylethylamine (0.45 mmol, 2.57 mmol) were added to a dichloromethane solution (20 mL) containing H-Val-Cit-PABC-MMAE (960 mg, 0.855 mmol). After overnight stirring, the reaction solution was purified with Sephadex LH20 (CHCl₃:MeOH 1:1) and by size exclusion high performance liquid chromatography to obtain Mal-PEG₁₂-Val-Cit-PABC-MMAE (769 mg, 48%) as a colorless oil.

Then, according to the general common technique, the antibody was reduced with mercaptoethylamine. The maleimide group of Mal-PEG₁₂-Val-Cit-PABC-MMAE and the thiol group of the antibody were linked by Michel addition reaction to obtain the ADC. The 669 antibody and a control antibody were used as antibodies.

### Example C15-2. Checking of Internalization of the Antibody

Wildtype DLD-1 cells were plated on a 96-well plate at 5,000 cells/well. The plate was incubated at 37° C. for 18 hours. After washing, a 488-labelled 669 antibody was added to the wells at 2 μL/well and was incubated at 37° C. for certain hours (1 hour, 3 hours, or 6 hours). After the wells were washed three times, a rabbit anti 488 antibody was added at 2 μL/well to stand still at 4° C. for 30 minutes to quench the fluorescence derived from the antibodies binding to the cell surface. After the wells were washed three times, 4% PFA was added at 100 μL/well to stand still at room temperature for 10 minutes. After the wells were washed three times, DAPI (1/500 dilution) was added to the wells at 100 μL/well to stand still at room temperature for 5 minutes. After the wells were washed three times, the wells were subjected to photographing or Array Scan analysis. An anti-EGFR antibody, Cetuximab (Erbitux) was used as a control. The results are shown in FIG. 25.

As shown in FIG. 25, it was observed that the 669 antibody was concentrated at a high concentration in the cells. On the contrary, the amount of internalized cetuximab was limited.

### Example C15-3. Cytotoxicity of the ADC

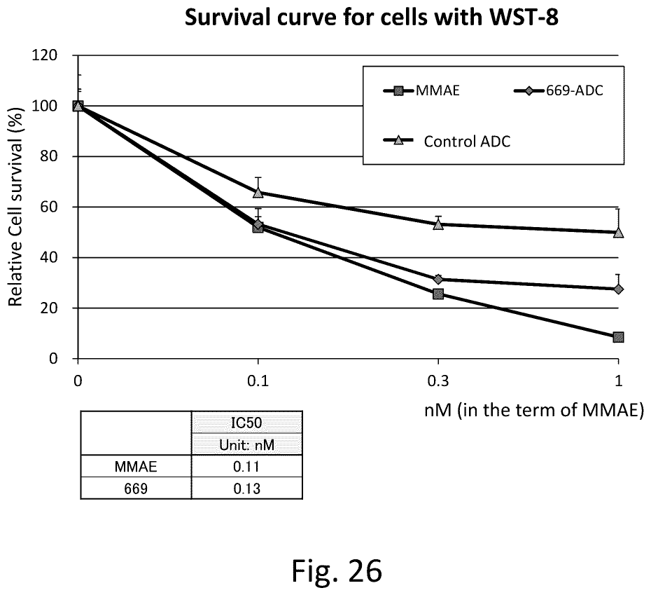

SW480 cells were plated on a 96-well plate at 2×10³ cells/well. On the next day, dilution series of MMAE (single agent), the ADC of 669 antibody, or the ADC of the control antibody (control ADC) were prepared with the concentrations in terms of MMAE. Three days later, WST-8, which is a cytotoxicity measuring agent, was added. After three hours, the cell viability (%) was checked. The results are shown in FIG. 26.

As shown in FIG. 26, it was shown that the ADC of 669 antibody exhibited the cytotoxicity to the cells as well as MMAE. Further, the ADC of 669 antibody exhibited a stronger cytotoxicity than the control ADC.

### Example C15-4. TMEM-180 Knockout Mice

TMEM-180 knockout mice were generated, and the phenotype thereof were checked.

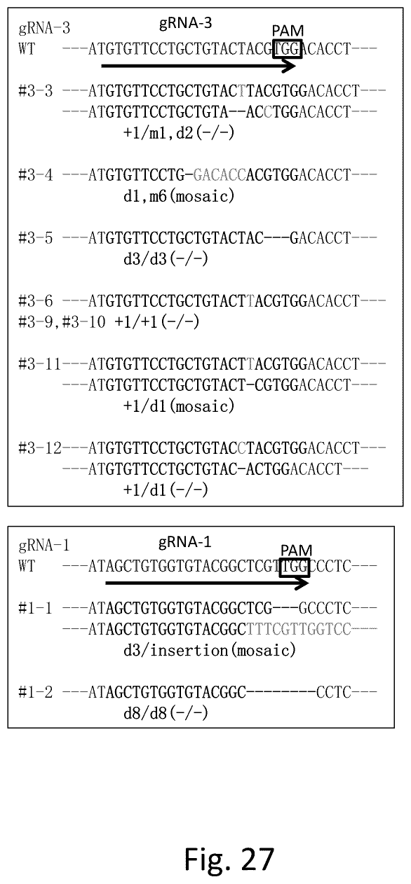

The TMEM-180 homo knockout mice were obtained by preparing a guide RNA at exon 2 in TMEM-180 by using CRISPR/Cas9 system and mating the obtained mice (see FIG. 27). It was confirmed that they are not embryonic lethal. This result suggests that the lack of the function of TMEM-180 will show little effect on normal tissues.

SEQ ID NOs: 1 to 3 respectively indicate the amino acid sequences of clone 98 heavy chain CDR1 to CDR3.

SEQ ID NOs: 4 to 6 respectively indicate the amino acid sequences of clone 98 light chain CDR1 to CDR3.

SEQ ID NOs: 7 to 9 respectively indicate the amino acid sequences of clone 101 heavy chain CDR1 to CDR3.

SEQ ID NOs: 10 to 12 respectively indicate the amino acid sequences of clone 101 light chain CDR1 to CDR3.

SEQ ID NOs: 13 and 14 respectively indicate the amino acid sequences of the heavy chain variable region and light chain variable region of clone 98.

SEQ ID NOs: 15 and 16 respectively indicate the amino acid sequences of the heavy chain variable region and light chain variable region of clone 101.

SEQ ID NO: 17 indicates the amino acid sequence of human TMEM-180 protein.

SEQ ID NOs: 18 to 21 respectively indicate primers (i) to (iv) for producing immunizing antigen 1.

SEQ ID NOs: 22 to 25 respectively indicate primers (i) to (iv) for producing immunizing antigen 2.

SEQ ID NOs: 26 to 31 indicate primers used for cloning an anti-TMEM-180 antibody.

SEQ ID NOs: 40 to 42 respectively indicate the amino acid sequences of clone 212 heavy chain CDR1 to CDR3.

SEQ ID NOs: 43 to 45 respectively indicate the amino acid sequences of clone 212 light chain CDR1 to CDR3.

SEQ ID NOs: 46 and 47 respectively indicate the amino acid sequences of the heavy chain variable region and light chain variable region of clone 212.

SEQ ID NOs: 48 to 50 respectively indicate the amino acid sequences of clone 129 heavy chain CDR1 to CDR3.

SEQ ID NOs: 51 to 53 respectively indicate the amino acid sequences of clone 129 light chain CDR1 to CDR3.

SEQ ID NOs: 54 and 55 respectively indicate the amino acid sequences of the heavy chain variable region and light chain variable region of clone 129.

SEQ ID NOs: 56 to 58 respectively indicate the amino acid sequences of clone 382 heavy chain CDR1 to CDR3.

SEQ ID NOs: 59 to 61 respectively indicate the amino acid sequences of clone 382 light chain CDR1 to CDR3.

SEQ ID NOs: 62 and 63 respectively indicate the amino acid sequences of the heavy chain variable region and light chain variable region of clone 382.

SEQ ID NOs: 64 to 66 respectively indicate the amino acid sequences of clone 1361 heavy chain CDR1 to CDR3.

SEQ ID NOs: 67 to 69 respectively indicate the amino acid sequences of clone 1361 light chain CDR1 to CDR3.

SEQ ID NOs: 70 and 71 respectively indicate the amino acid sequences of the heavy chain variable region and light chain variable region of clone 1361.

SEQ ID NOs: 72 to 74 respectively indicate the amino acid sequences of clone 669 heavy chain CDR1 to CDR3.

SEQ ID NOs: 75 to 77 respectively indicate the amino acid sequences of clone 669 light chain CDR1 to CDR3.

SEQ ID NOs: 78 and 79 respectively indicate the amino acid sequences of the heavy chain variable region and light chain variable region of clone 669.

SEQ ID NOs: 80 to 82 respectively indicate the amino acid sequences of clone 699 heavy chain CDR1 to CDR3.

SEQ ID NOs: 83 to 85 respectively indicate the amino acid sequences of clone 699 light chain CDR1 to CDR3.

SEQ ID NOs: 86 and 87 respectively indicate the amino acid sequences of the heavy chain variable region and light chain variable region of clone 699.

SEQ ID NOs: 88 to 90 respectively indicate the amino acid sequences of clone 1052 heavy chain CDR1 to CDR3.

SEQ ID NOs: 91 to 93 respectively indicate the amino acid sequences of clone 1052 light chain CDR1 to CDR3.

SEQ ID NOs: 94 and 95 respectively indicate the amino acid sequences of the heavy chain variable region and light chain variable region of clone 1052.

SEQ ID NOs: 96 to 98 respectively indicate the amino acid sequences of clone 1105 heavy chain CDR1 to CDR3.

SEQ ID NOs: 99 to 101 respectively indicate the amino acid sequences of clone 1105 light chain CDR1 to CDR3.

SEQ ID NOs: 102 and 103 respectively indicate the amino acid sequences of the heavy chain variable region and light chain variable region of clone 1105.

SEQ ID NOs: 104 to 150 indicate the amino acid sequences of TMEM-180-derived peptides binding to HLA type A2 as predicted by HLA Peptide Binding Predictions.

SEQ ID NOs: 151 to 170 indicate the amino acid sequences of TMEM-180-derived peptides binding to HLA type A24 as predicted by HLA Peptide Binding Predictions.

SEQ ID Nos: 171 to 173 indicate the amino acid sequences of clone 1361-5 heavy chain CDR 1 to 3, respectively.

SEQ ID Nos: 174 to 176 indicate the amino acid sequences of clone 1361-5 light chain CDR 1 to 3, respectively.

SEQ ID NOs: 177 and 178 indicate the amino acid sequences of the heavy chain variable region and light chain variable region of clone 1361-5, respectively

SEQ ID Nos: 179 and 180 indicate the nucleic acid sequence and amino acid sequence of clone 1361-5 heavy chain, respectively.

SEQ ID Nos: 181 and 182 indicate the nucleic acid sequence and amino acid sequence of clone 1361-5 light chain, respectively.

SEQ ID Nos: 183 and 184 indicate the nucleic acid sequences of the forward primer and reverse primer used for the introduction of Y79A mutation in 1361-5 clone.

SEQ ID Nos: 185 and 186 indicate the nucleic acid sequences of the forward primer and reverse primer used for the introduction of S82A mutation in 1361-5 clone.

SEQ ID Nos: 187 to 192 indicate the nucleic acid sequences of the forward primers and reverse primers used for the introduction of site specific mutations into the Fc region of 1361-5 antibody as described in Example B11-2.

1-16. (canceled)

17. A cancer testing method, comprising a step of detecting the amount of exosomes having TMEM-180 in the body fluid sample obtained from a subject.

18. The cancer testing method according to claim 17, wherein the step of detecting the amount of exosomes having TMEM-180 is conducted with an antibody binding to TMEM-180 or an antigen-binding fragment thereof.

19. The method according to claim 18, wherein the step of detecting the amount of exosomes having TMEM-180 is conducted with the antibody or an antigen-binding fragment thereof, wherein the antibody and the antigen-binding fragment thereof comprises:

20. (canceled)

21. A method of treating a subject, comprising administering to the subject an antibody that binds to TMEM-180 expressed on a surface of cancer, wherein the subject has been determined to have exosomes expressing TMEM-180 on their surface in a body fluid sample obtained from the human subject.

22. The method of claim 21, wherein the subject has colon cancer.
